(19) United States
(12) Patent Application Publication
(10) Pub. No.: US 2018/0140722 A1
(43) Pub. Date: May 24, 2018

(54) METHODS AND COMPOSITIONS FOR TREATMENT OF HEART FAILURE

(71) Applicant: The University of North Carolina at Chapel Hill, Chapel Hill, NC (US)

(72) Inventors: Monte Willis, Chapel Hill, NC (US); Scott Bultman, Chapel Hill, NC (US)

(21) Appl. No.: 15/564,531

(22) PCT Filed: Apr. 6, 2016
(86) PCT No.: PCT/US2016/026247

§ 371 Date: Feb. 8, 2018

Related U.S. Application Data
(60) Provisional application No. 62/143,348, filed on Apr. 6, 2015.

(51) Int. Cl. A61K 49/00

(52) U.S. Cl. CPC A61K 49/0008

(57) ABSTRACT

The present invention relates to methods and compositions for treating heart failure in a subject using histone deacetylase (HDAC) inhibitors and/or histone acetyltransferases (HAT) activators.

### STATEMENT OF PRIORITY

This application is a 35 U.S.C. § 371 national phase application of International Application Serial No. PCT/US2016/026247, filed Apr. 6, 2016, which claims the benefit, under 35 U.S.C. § 119(e), of U.S. Provisional Application Ser. No. 62/143,348, filed Apr. 6, 2015, the entire contents of each of which are incorporated by reference herein.

### STATEMENT OF FEDERAL SUPPORT

This invention was made with government support under Grant Nos. HL104129 (R01) and CA125237 awarded by the National Institutes of Health. The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-739_ST25.txt, 24,451 bytes in size, generated on Feb. 8, 2018 and filed via EFS-Web, is provided in lieu of a paper copy. This Sequence Listing is hereby incorporated by reference into the specification for its disclosures.

### FIELD OF THE INVENTION

The present invention relates to methods and compositions for treating heart failure in a subject using histone deacetylase (HDAC) inhibitors and histone acetyltransferases (HAT) activators.

### BACKGROUND OF THE INVENTION

SWI/SNF chromatin-remodeling complexes are recruited by pioneer transcription factors to the enhancers and promoters of target genes where they alter the position of nucleosomes in an ATP-dependent manner to positively or negatively regulate transcription. Despite the well-known diversity of mammalian SWI/SNF complexes, which have many different subunit compositions, they utilize either BRG1 or BRM (also known as SMARCA4 and SMARCA2, respectively) as their sole catalytic subunit with DNA-dependent ATPase activity. Mouse knockout studies have demonstrated that BRG1-catalyzed complexes are required for cardiomyocyte development but are dispensable in adult cardiomyocytes. In the developing embryo, BRG1 physically interacts with the cardiogenic transcription factors TBX5, GATA4, and NKX2.5 (mediated via the SWI/SNF subunit BAF60c) and regulates the expression of genes necessary for cardiomyocyte proliferation and differentiation. For example, BRG1 interacts with HDACs (histone deacetylases) and PARPs (poly-ADP ribose polymerases) to activate the fetal β myosin heavy chain (MHC) isoform while repressing the postnatal αMHC isoform. BRG1 is required in embryonic cardiomyocytes for organismal survival, but this is not the case for adult cardiomyocytes based on an inducible knockout that does not confer an overt phenotype under basal conditions. However, increased BRG1 expression in adult cardiomyocytes does promote cardiac hypertrophy induced by transverse aortic constriction (TAC) to pressure overload the heart. Under these conditions, BRG1 is required for an αMHC-to-βMHC shift, which is part of a larger program to reactivate several fetal cardiac genes including brain natriuretic peptide (BNP) and atrial natriuretic factor (ANF) during heart failure (HF). In contrast to BRG1, BRM-catalyzed complexes are dispensable in both developing and adult cardiomyocytes based on the viability of Brm-deficient mice.

The coordinated contraction of the heart relies on the transmission of the pacemaker impulses generated at the sinoatrial (SA) node proceeding from the atrial to ventricular myocardium through a network of cells comprising the conduction system. The cells of the conduction system are of cardiomyocyte origin, and multiple transcription factors have been identified that are required to maintain their function, including NKX2.5, SHOX, and TBX5.

These transcription factors maintain critical ion channels responsible for the propagation of electrical signals. For example, the lack of the NKX2.5 and Homeodomain-Only Protein (HOP) results in decreased expression of cardiac connexins such as CX40, which are required for cell-to-cell electrical communication. These perturbations result in defective electrical signal conduction as manifested by prolonged PR intervals, widened QRS, prolonged QT intervals, and sudden death. Loss of CX40 may be a major mechanism underlying the cardiac defects in HOP deficiency, paralleling clinically relevant human conduction system disorders and dilated cardiomyopathy. An important challenge is to identify which chromatin-modifying factors and epigenetic mechanisms are required for cardiac conduction.

The primary therapies for cardiac arrhythmias act by interfering with the sodium channel activity (Class 1), potassium efflux (Class 3), calcium channels and the AV node (Class 4), or beta adrenergic receptor blockade (Class II). While the expression of these ion channels is known to be decreased in heart failure, as are other gap junction proteins critical to cardiomyocyte conduction (e.g., Cx40, Cx43), no therapies exist to regulate this unknown process.

The present invention overcomes these shortcomings by providing treatments for heart failure and related diseases.

### SUMMARY OF THE INVENTION

The present invention is based on the identification of the role of Brg1 and/or Brm expression in heart failure and related diseases, and the effectiveness of treating these diseases by delivery of histone deacetylase (HDAC) inhibitor and/or a histone acetyltransferase (HAT) activator.

Accordingly, in one aspect, the invention relates to a method of increasing expression of Brg1 and/or Brm mRNA and protein in a subject, comprising delivering to the subject an effective amount of a histone deacetylase (HDAC) inhibitor and/or a histone acetyltransferase (HAT) activator, thereby increasing expression of Brg1 and/or Brm mRNA and/or protein in the subject.

A further aspect of the invention relates to a method of decreasing expression of c-Myc mRNA and/or protein in a subject, comprising delivering to the subject an effective amount of a histone deacetylase (HDAC) inhibitor and/or a histone acetyltransferase (HAT) activator, thereby decreasing expression of c-Myc mRNA and/or protein in the subject.

Another aspect of the invention relates to a method of increasing expression of Cx40, Cx43, and/or Scn5a mRNA and/or protein in a subject, comprising delivering to the subject an effective amount of a histone deacetylase (HDAC) inhibitor and/or a histone acetyltransferase (HAT) activator, thereby increasing expression of Cx40, Cx43, and/or Scn5a mRNA and/or protein in the subject.

An additional aspect of the invention relates to a method of increasing expression of Brg1, Brm, Cx40, Cx43, and/or Scn5a mRNA and/or protein and/or decreasing expression of c-Myc in a subject, comprising delivering to the subject an effective amount of a histone deacetylase (HDAC) inhibitor and/or a histone acetyltransferase (HAT) activator, thereby increasing expression of Brg1, Brm, Cx40, Cx43, and/or Scn5a mRNA and/or protein and/or decreasing expression of c-Myc mRNA and/or protein in the subject.

A further aspect of the invention relates to a method of increasing and/or improving conduction in the heart in a subject in need thereof, comprising increasing the expression of Brg1 and/or Brm mRNA and/or protein in the subject, thereby increasing and/or improving conduction in the heart in the subject.

Another aspect of the invention relates to a method of increasing and/or improving conduction in the heart in a subject in need thereof, comprising decreasing expression of c-Myc mRNA and/or protein in the subject, thereby increasing and/or improving conduction in the heart in the subject.

An additional aspect of the invention relates to a method of increasing and/or improving conduction in the heart in a subject in need thereof, comprising increasing expression of Cx40, Cx43, and/or Scn5a mRNA and/or protein in the subject, thereby increasing and/or improving conduction in the heart in the subject.

A further aspect of the invention relates to a method of increasing and/or improving conduction in the heart in a subject in need thereof, comprising increasing expression of Brg1, Brm, Cx40, Cx43, and/or Scn5a mRNA and/or protein and/or decreasing expression of c-Myc mRNA and/or protein in the subject, thereby increasing and/or improving conduction in the heart in the subject.

A still further aspect of the invention relates to a method of treating and/or preventing a cardiac conduction defect in a subject in need thereof, comprising increasing the expression of Brg1 and/or Brm mRNA and/or protein in the subject, thereby treating and/or preventing the cardiac conduction defect in the subject.

An additional aspect of the invention relates to a method of treating and/or preventing a cardiac conduction defect in a subject in need thereof, comprising decreasing the expression of c-Myc mRNA and/or protein in the subject, thereby treating and/or preventing the cardiac conduction defect in the subject.

A further aspect of the invention relates to a method of treating and/or preventing a cardiac conduction defect in a subject in need thereof, comprising increasing the expression of Cx40, Cx43, and/or Scn5a mRNA and/or protein in the subject, thereby treating and/or preventing the cardiac conduction defect in the subject.

An additional aspect of the invention relates to a method of treating and/or preventing a cardiac conduction defect in a subject in need thereof, comprising increasing expression of Brg1, Brm, Cx40, Cx43, and/or Scn5a mRNA and/or protein and/or decreasing expression of c-Myc mRNA and/or protein in the subject, thereby treating and/or preventing a cardiac conduction defect in the subject.

A further aspect of the invention relates to a method of treating and/or preventing arrhythmia in a subject in need thereof, comprising increasing the expression of Brg1 and/or Brm mRNA and/or protein in the subject, thereby treating and/or preventing arrhythmia in the subject.

A still further aspect of the invention relates to a method of treating and/or preventing arrhythmia in a subject in need thereof, comprising decreasing the expression of c-Myc mRNA and/or protein in the subject, thereby treating and/or preventing arrhythmia in the subject.

Another aspect of the invention relates to a method of treating and/or preventing arrhythmia in a subject in need thereof, comprising increasing the expression of Cx40, Cx43, and/or Scn5a mRNA and/or protein in the subject, thereby treating and/or preventing arrhythmia in the subject.

A further aspect of the invention relates to a method of treating and/or preventing arrhythmia in a subject in need thereof, comprising increasing expression of Brg1, Brm, Cx40, Cx43, and/or Scn5a mRNA and/or protein and/or decreasing expression of c-Myc mRNA and/or protein in the subject, thereby treating and/or preventing arrhythmia in the subject.

An additional aspect of the invention relates to a method of treating and/or preventing heart failure in a subject in need thereof, comprising increasing the expression of Brg1 and/or Brm mRNA and/or protein in the subject, thereby treating and/or preventing heart failure in the subject.

A further aspect of the invention relates to a method of treating and/or preventing heart failure in a subject in need thereof, comprising decreasing the expression of c-Myc mRNA and/or protein in the subject, thereby treating and/or preventing heart failure in the subject.

A still further aspect of the invention relates to a method of treating and/or preventing heart failure in a subject in need thereof, comprising increasing the expression of Cx40, Cx43, and/or Scn5a mRNA and/or protein in the subject, thereby treating and/or preventing heart failure in the subject.

Another aspect of the invention relates to a method of treating and/or preventing heart failure in a subject in need thereof, comprising increasing expression of Brg1, Brm, Cx40, Cx43, and/or Scn5a mRNA and/or protein and/or decreasing expression of c-Myc mRNA and/or protein in the subject, thereby treating and/or preventing heart failure in the subject.

An additional aspect of the invention relates to a method of treating and/or preventing familial hypertrophic cardiomyopathy in a subject in need thereof, comprising increasing the expression of Brg1 and/or Brm mRNA and/or protein in the subject, thereby treating and/or preventing familial hypertrophic cardiomyopathy in the subject.

Another aspect of the invention relates to a method of treating and/or preventing familial hypertrophic cardiomyopathy in a subject in need thereof, comprising decreasing the expression of c-Myc mRNA and/or protein in the subject, thereby treating and/or preventing familial hypertrophic cardiomyopathy in the subject.

A further aspect of the invention relates to a method of treating and/or preventing familial hypertrophic cardiomyopathy in a subject in need thereof, comprising increasing the expression of Cx40, Cx43, and/or Scn5a mRNA and/or protein in the subject, thereby treating and/or preventing familial hypertrophic cardiomyopathy in the subject.

A still further aspect of the invention relates to a method of treating and/or preventing familial hypertrophic cardiomyopathy in a subject in need thereof, comprising increasing expression of Brg1, Brm, Cx40, Cx43, and/or Scn5a mRNA and/or protein and/or decreasing expression of c-Myc mRNA and/or protein in the subject, thereby treating and/or preventing familial hypertrophic cardiomyopathy in the subject.

An additional aspect of the invention relates to a method of treating and/or preventing long QT syndrome (LQTS) in a subject in need thereof, comprising increasing the expression of Brg1 and/or Brm mRNA and/or protein in the subject, thereby treating and/or preventing long QT syndrome in the subject.

Another aspect of the invention relates to a method of treating and/or preventing long QT syndrome in a subject in need thereof, comprising decreasing the expression of c-Myc mRNA and/or protein in the subject, thereby treating and/or preventing long QT syndrome in the subject.

A further aspect of the invention relates to a method of treating and/or preventing long QT syndrome in a subject in need thereof, comprising increasing the expression of Cx40, Cx43, and/or Scn5a mRNA and/or protein in the subject, thereby treating and/or preventing long QT syndrome in the subject.

A still further aspect of the invention relates to a method of treating and/or preventing long QT syndrome in a subject in need thereof, comprising increasing expression of Brg1, Brm, Cx40, Cx43, and/or Scn5a mRNA and/or protein and/or decreasing expression of c-Myc mRNA and/or protein in the subject, thereby treating and/or preventing long QT syndrome in the subject.

In additional aspects, the methods of increasing and/or improving conduction in the heart, treating and/or preventing a cardiac conduction defect, arrhythmia, heart failure, familial hypertrophic cardiomyopathy, and/or long QT syndrome in a subject in need thereof, comprises delivering to the subject an effective amount of a histone deacetylase (HDAC) inhibitor and/or a histone acetyltransferase (HAT) activator.

### BRIEF DESCRIPTION OF THE DRAWINGS

FIG. 1A-1C. Tamoxifen-induced ablation of Brg1 in cardiomyocytes. FIG. 1A, PCR detection of the Brg1 floxed (fl, top panel) and Δfloxed (Δfl, bottom panel) alleles in genomic DNA prepared from cardiac tissue of Brg1^{fl/fl}; MHC-Cre-ERT mice that were untreated (−) or treated (+) with tamoxifen (TAM). The floxed PCR product is diminished but still detected in TAM-treated mice because the Cre-mediated excision event does not occur in cell types other than cardiomyocytes such as vascular endothelial cells. NTC, no template control. FIG. 1B, IHC showing BRG1 nuclear staining in cardiomyocytes (encircled region) and vascular endothelial cells (arrow) of cardiac tissue sections from control mice (Group 2: Brg1^{fl/fl}; no MHC-Cre-ERT transgene but treated with TAM). FIG. 1C, immunohistochemistry (IHC) showing lack of BRG1 staining in cardiomyocytes (encircled region) in cardiac tissue sections from Brg1/Brm double-mutant mice. Presence of BRG1 staining in vascular endothelial cells (arrow) at levels comparable to the control serves as an internal positive control.

FIG. 2A-2F. Brg1/Brm double mutants undergo arrhythmias, rapid heart failure, and death. FIG. 2A, Echocardiogram-based measurements of ejection fraction % and fractional shortening % in control groups and Brg1/Brm double mutants at baseline (prior to tamoxifen) and at 1-day pre-mortem. FIG. 2B, Six panels show left-ventricle morphometrics and heart rate, as indicated, with first 5 histograms representing baseline measurements and last 2 histograms representing 1-day pre-mortem measurements. FIG. 2C, The ejection fraction % and fractional shortening % data at 1-day pre-mortem from panel (A) are reproduced at the left. These data are juxtaposed with 1-day pre-mortem data from the double mutants separated out into two subsets (highlighted by arrows and histograms labeled Subset 1 and Subset 2) where the phenotypes differed with respect to wall thickening, LV dilation, and systolic dysfunction, but not heart rate. FIG. 2D, Six panels show left-ventricle morphometrics and heart rate that are the same as panel (B) except only 1-day pre-mortem data are shown and the double mutant data are shown combined and separated out into the two subgroups as indicated. For FIGS. 2A-2D, the data represent means±SEM with the number of mice per group indicated in each histogram. One-Way Analysis of Variance was performed followed by an all pair-wise multiple comparison procedure (Holm-Sidak method) with significant differences indicated (§, p<0.001 vs. all other groups; *p<0.001 vs. all other groups; **p<0.05 vs. Column 1; †p<0.01 vs. Column 4). FIG. 2E, Kaplan-Meier survival curve of mice after administration of tamoxifen (+TAM) on days 1 through 7. The number of mice for each genotype and ± TAM treatment is listed below. FIG. 2F, ECG data from control and double mutant mice at baseline (prior to tamoxifen) and at days 13 and 17 post-tamoxifen induction that includes measurements in the hours preceding death.

FIG. 3A-3B. Cardiomyocyte vacuolization and induction of β-MHC in Brg1/Brm double mutants. FIG. 3A, Representative H&E- and Mason's Trichrome-stained heart sections from Brg1/Brm double mutant and control mice as indicated. FIG. 3B, RT-qPCR analysis of fetal genes that are induced during cardiac hypertrophy at early (left) and late (right) time points. Results are presented as means±SEM based on 5 independent experiments with significant differences indicated (*, p<0.05; **, p<0.01).

FIG. 4A-4E. Brg1/Brm double-mutant hearts undergo autophagy. FIG. 4A, Representative TEM images of double-mutant hearts at day 14 (post-tamoxifen induction) with degenerating mitochondria and autophagic vesicles indicated. FIG. 4B, Representative TEM images of control hearts at day 14 (post-tamoxifen induction), which lack evidence of autophagy. FIG. 4C, Top, representative Western blot of LC3B isoforms in heart tissue from bafilomycin-treated double-mutant and control mice at day 15 post-tamoxifen induction. Bottom, quantification of the LC3BII/LC3BI ratio based on densitrometry of above immunoblot. FIG. 4D, Representative Western blot of Beclin in same heart samples from panel (C) with Tubulin serving as a loading control. FIG. 4E, RT-qPCR analysis of autophagy genes at day 15 post-tamoxifen induction. Data are normalized to Gapdh and represent means±SEM based on 5 independent experiments with significant differences indicated (*p<0.01).

FIG. 5A-5F. Altered mitochondrial dynamics in Brg1/Brm double-mutant hearts. FIG. 5A, Representative TEM images of double-mutant and control heart sections. FIG. 5B, Quantification of mitochondrial area and size based on measurements from TEM images. Data were obtained from 3 control mice and 3 double-mutant mice with >6 fields from each animal. 1,529 control mitochondria and 2,380 double-mutant mitochondria were scored. FIG. 5C, Average mitochondria area and number±SEM based on data from panel (B). *p<0.05. FIG. 5D, qPCR of 3 mitochondria-encoded loci normalized to nuclear H19 locus in control and double-mutant hearts at day 14 post-tamoxifen induction. Data are presented as means±SEM based on 5 independent experiments with significant differences indicated (*, p<0.01). FIG. 5E, RT-qPCR analysis of Mfn1 and Opa1 fusion genes normalized to BestKeeper Index in control and double-mutant hearts at day 15 post-tamoxifen. Data are presented as means±SEM based on 5 independent experiments with significant differences indicated (*, p<0.01). FIG. 5F, RT-qPCR analysis of Fis1 and Drp1 fission genes normalized to BestKeeper Index in control and double-mutant hearts at day 15 post-tamoxifen induction. Data are presented as means±SEM based on 5 independent experiments with significant differences indicated (*, p<0.01).

FIG. 6A-6D. Brg1/Brm double-mutant hearts have increased protein aggregation and undergo the unfolded protein response. FIG. 6A, Quantification of unfolded protein accumulation in control and double-mutant hearts at two time points (days 9 and 15 post-tamoxifen induction). Data are presented as means±SEM based on 5 independent experiments with significant differences indicated (*, p<0.01). FIG. 6B, Quantification of pre-amyloid oligomers in control and double-mutant hearts at day 15 post-tamoxifen induction. Data are presented as means±SEM based on 5 independent experiments. n.s., not significant. FIG. 6C, Representative IHC image of GRP78 staining in control and double-mutant hearts at day 15 post-tamoxifen induction. FIG. 6D, RT-qPCR analysis of genes involved in the unfolded protein response normalized to Gapdh in day 15 hearts from control and double-mutant mice. Data are presented as means±SEM based on 5 independent experiments with significant differences indicated (*, p<0.05).

FIG. 7A-7F. A link between BRG1/BRM and c-Myc in arrhythmias and heart failure. FIG. 7A, Hierarchal clustering of gene expression profiles from 3 control groups and Brg1/Brm double-mutant hearts at day 9 post-tamoxifen induction. FIG. 7B, TRANSFAC analysis of differentially expressed genes showing significant enrichment of c-Myc-binding sites in promoter regions of differentially regulated genes. FIG. 7C, RT-qPCR demonstrating that c-Myc is expressed in an inducible transgenic mouse line within 24 hours of DOX-mediated induction. Data are normalized to Gapdh and presented as means±SEM based on 5 independent experiments with significant differences indicated (*, p<0.05). FIG. 7D, RT-qPCR of Cx43 in transgenic mouse model that inducibly expresses c-Myc prior to induction (MYC-OFF) and after DOX-mediated induction (MYC-ON, Day 1 and Day 2). Data are normalized to Gapdh and presented as means±SEM based on 5 independent experiments with significant differences indicated (*, p<0.05). FIG. 7E, Representative Western blot of CX43 expression in transgenic mouse model that inducibly expresses c-Myc prior to induction (MYC-OFF) and after DOX-mediated induction (MYC-ON, Day 3). Actin serves as a loading control. 3 independent samples for MYC-OFF and MCY-ON are shown. FIG. 7F, ECG sample trace readings from 3 MYC-OFF controls and 4 MYC-ON mice showing Wenckebach second-degree heart block by 3 days of DOX-induced c-MYC induction and a complete heart block by day 6.

FIG. 8A-8E. BRG1/BRM regulates the expression of c-Myc, cardiac connexins, and Tbx5. FIG. 8A, RT-qPCR analysis of c-Myc normalized to Gapdh in control and double-mutant hearts at day 15 post-tamoxifen induction. Data are presented as means±SEM based on 5 independent experiments with significant differences indicated (*, p<0.05). FIG. 8B, RT-qPCR analysis of Cx40, Cx43, and Scn5a normalized to Gapdh in control and double-mutant hearts at day 15 post-tamoxifen. Data are presented as means±SEM based on 5 independent experiments with significant differences indicated (*, p<0.05). FIG. 8C, RT-qPCR analysis of Tbx5 and Nkx2. 5 normalized to Gapdh in control and double-mutant hearts at day 15 post-tamoxifen. Data are presented as means±SEM based on 5 independent experiments with significant differences indicated (*, p<0.05). FIG. 8D, Quantitative ChIP assays demonstrating BRG1 occupancy of Cx40, Cx43, and Scn5a promoters in wild-type mouse heart tissue. As a negative control, the same heart tissues were immunoprecipitated with IgG but processed identically. Histograms show the relative enrichment by comparing each ChIP sample to input by qPCR (means±SEM for three independent samples). FIG. 8E, Final model. The BRG1 and BRM catalytic subunits of SWI/SNF complexes directly and indirectly activate the expression of Cx40, Cx43, and Scn5a to facilitate conduction in cardiomyocytes. The direct regulation is based on ChIP assays demonstrating BRG1 occupancy at each promoter. The indirect regulation is mediated by activation of Tbx5 (an activator) and inhibition of c-Myc (an inhibitor). BRG1/BRM and/or properly regulated conduction are necessary for mitochondrial fusion/fission, mitophagy, and the UPR to maintain cardiomyocyte homeostasis.

FIG. 9. Cardiac function of individual Brm−−/flx/flx No Tg Chow Diet (Group 1) over time course after the start of TAM feeding. Data are based on serial echocardiography on conscious mice. N=3 waveforms per time point.

FIG. 10. Cardiac function of individual Brm−/−flx/flx No Tg+TAM (Group 2) over time course after the start of TAM feeding. Data are based on serial echocardiography on conscious mice. N=3 waveforms per time point.

FIG. 11. Cardiac function of individual Brm−/−flx/flx Brg1 Tg+Chow (Group 3) over time course after the start of TAM feeding. Data are based on serial echocardiography on conscious mice. N=3 waveforms per time point.

FIG. 12. Cardiac function of individual Brm−/−flx/flx Brg1 Tg++TAM (Group 4) over time course after the start of TAM feeding. Data are based on serial echocardiography on conscious mice. N=3 waveforms per time point.

FIG. 13. Cardiac function of individual Brm−/−flx/+Brg1 Tg+Tam (Group 5) over time course after the start of TAM feeding. Data are based on serial echocardiography on conscious mice. N=3 waveforms per time point.

FIG. 14A-14D. Histological analysis of Brg1/Brm double-mutant hearts. A, Control Group 6 Brm−/−flx/+Brg1 Tg++Tam; B-D, Individuals hearts from Group 4 Brg1/Brm double-mutant mice.

FIG. 15A-15C. RT-qPCR analysis of autophagy genes (A, Atg5; B, Bnip3; C, Vps34) in control and double-mutant hearts at day 10 post-tamoxifen induction. Data are normalized to Gapdh and presented as means±SEM based on 5 independent experiments with significant differences indicated (*p<0.05). See FIG. 4E for analysis at later (day 15) time point.

FIG. 16A-16D. RT-qPCR analysis of mitochondrial fusion and fission genes (A, Drp1; B, Fis1; C, Mfn1; D, Opa1) in control and double-mutant hearts at day 10 post-tamoxifen induction. Data are normalized to Gapdh or 18S and presented as means±SEM based on 5 independent experiments with no significant differences. See FIGS. 5E and 5F for analysis at later (day 15) time point.

FIG. 17A-17B. RT-qPCR analysis of genes involved in the unfolded protein response normalized to Gapdh in day 15 hearts from control and double-mutant mouse hearts. Data are presented as means±SEM based on 5 independent experiments with significant differences indicated (*, p<0.05). A, Down-regulated genes (Ire-1a, Atf6a, Grp78). B, Genes not significantly changed (Atf4, Fabp4, Pparg, Pref1).

FIG. 18. ECG-based measurements from 3 MYC-OFF controls and 4 MYC-ON mice at three time points relative to DOX-mediated induction. The plots show significant differences (*, p<0.05) in heart rate, PR interval, QRS duration, and QTc.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination. For example, features described in relation to one embodiment may also be applicable to and combinable with other embodiments and aspects of the invention. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all terms, including technical and scientific terms used in the description, have the same meaning as commonly understood by one of ordinary skill in the art to which this invention belongs.

All patents, patent applications, patent publications and non-patent publications referred to herein are incorporated by reference in their entirety. In case of a conflict in terminology, the present specification is controlling.

The terminology used in the description of the invention herein is for the purpose of describing particular embodiments only and is not intended to be limiting of the invention. As used in the description of the embodiments of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items.

Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “consists essentially of” (and grammatical variants), as applied to the compositions of this invention, means the composition can contain additional components as long as the additional components do not materially alter the composition. The term “materially altered,” as applied to a composition, refers to an increase or decrease in the therapeutic effectiveness of a composition of at least about 20% or more as compared to the effectiveness of a composition consisting of the recited components. Thus, the term “consisting essentially of” when used in a claim of this invention is not intended to be interpreted to be equivalent to “comprising.”

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the terms “increase,” “increases,” “increased,” “increasing,” and similar terms indicate an elevation of at least about 25%, 50%, 75%, 100%, 150%, 200%, 300%, 400%, 500% or more. Thus, for example, increasing the expression of Brg1, Brm, Cx40, Cx43, and/or Scn5a in a subject means increasing expression of said gene by at least about 200% to about 400% (e.g., 200, 225, 250, 275, 300, 325, 350, 375, 400%, and the like, and any value or range therein) or more as compared to the level of the same gene in the subject prior to administration of the HDAC inhibitor and/or the HAT activator (or as compared to the levels as observed in heart failure and related disorders as described herein).

The terms “decrease,” “inhibit” or “reduce” or grammatical variations thereof as used herein refer to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%). Thus, for example, decreasing or reducing the expression of c-Myc in a subject means decreasing expression of said gene by about 50% as compared to the level of the same gene in the subject prior to administration of the HDAC inhibitor and/or the HAT activator (or as compared to the levels as observed in heart failure and related disorders as described herein).

An “effective” amount as used herein is an amount that provides a desired effect.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject. Determination of a therapeutically effective amount, as well as other factors related to effective administration of a compound of the present invention to a subject of this invention, including dosage forms, routes of administration, and frequency of dosing, may depend upon the particulars of the condition that is encountered, including the subject and condition being treated or addressed, the severity of the condition in a particular subject, the particular compound being employed, the particular route of administration being employed, the frequency of dosing, and the particular formulation being employed. Determination of a therapeutically effective treatment regimen for a subject of this invention is within the level of ordinary skill in the medical or veterinarian arts. In clinical use, an effective amount may be the amount that is recommended by the U.S. Food and Drug Administration, or an equivalent foreign agency. The amount of active ingredient that can be combined with the carrier materials to produce a single dosage form varies depending upon the subject being treated and the particular mode of administration.

By the terms “treat,” “treating,” or “treatment,” it is intended that the severity of the subject's condition is reduced or at least partially improved or modified and that some alleviation, mitigation or decrease in at least one clinical symptom is achieved.

The terms “prevent,” “preventing,” and “prevention” (and grammatical variations thereof) refer to a delay in the extent or severity of a disease, disorder and/or clinical symptom(s) after onset relative to what would occur in the absence of carrying out the methods of the invention prior to the onset of the disease, disorder and/or clinical symptom(s). The prevention can be complete, e.g., the total absence of the infection, disease, condition and/or clinical symptom(s). The prevention can also be partial, such that the occurrence of the disease, disorder and/or clinical symptom(s) in the subject and/or the severity of onset is less than what would occur in the absence of the present invention. Thus, the terms “prevent,” “preventing,” and “prevention” and like terms are used herein to include imparting any level of prevention or protection which is of some benefit to a subject, such that there is a reduction in the incidence and/or the severity of the disease in a treated subject, regardless of whether the protection or reduction in incidence and/or severity is partial or complete. In some embodiments of the invention, preventing can mean reducing the risk of a cardiac conduction defect, arrhythmia heart failure, familial hypertrophic cardiomyopathy and/or long QT syndrome.

“Concurrently” means sufficiently close in time to produce a combined effect (that is, concurrently can be simultaneously, or it can be two or more events occurring within a short time period before or after each other). In some embodiments, the administration of two or more compounds “concurrently” means that the two compounds are administered closely enough in time that the presence of one alters the biological effects of the other. The two compounds can be administered in the same or different formulations or sequentially. Concurrent administration can be carried out by mixing the compounds prior to administration, or by administering the compounds in two different formulations, for example, at the same point in time but at different anatomic sites or using different routes of administration.

“Pharmaceutically acceptable” as used herein means that the compound or composition is suitable for administration to a subject to achieve the treatments described herein, without unduly deleterious side effects in light of the severity of the disease and necessity of the treatment.

A “subject” as used herein includes any animal in which treatment or prevention of cardiac conduction defect, arrhythmia, heart failure, familial hypertrophic cardiomyopathy and/or long QT syndrome is desired. A subject of this invention can be a subject in need of treatment and/or prevention of cardiac conduction defect, arrhythmia, heart failure, familial hypertrophic cardiomyopathy and/or long QT syndrome; that is a subject known to have, suspected of having, or at increased risk of developing or having cardiac conduction defect, arrhythmia, heart failure, familial hypertrophic cardiomyopathy and/or long QT syndrome. Thus, in some embodiments, the subject of the methods of this invention can be at risk of having cardiac conduction defect, arrhythmia, heart failure, familial hypertrophic cardiomyopathy and/or long QT syndrome, experiencing cardiac conduction defect, arrhythmia, heart failure, familial hypertrophic cardiomyopathy and/or long QT syndrome, or have previously experienced cardiac conduction defect, arrhythmia, heart failure, familial hypertrophic cardiomyopathy and/or long QT syndrome.

A subjects can include an animal subject, particularly a mammalian subject such as a canine, feline, bovine, caprine, equine, ovine, porcine, a rodent (e.g., rat and mouse), a lagomorph, a primates (including a non-human primate), etc., for veterinary medicine or pharmaceutical drug development purposes. In some embodiments, a subject of this invention can be an animal model of cardiac conduction defect, arrhythmia, heart failure, familial hypertrophic cardiomyopathy and/or long QT syndrome. In some embodiments, a subject of this invention can be a mammalian subject, which can be a human subject. A human subject of this invention can be male or female and may be of any race or ethnicity, including, but not limited to, Caucasian, Black, African-American, African, Asian, Hispanic, Indian, etc. A subject may be of any age, including newborn, neonate, infant, child, juvenile, adolescent, adult, and/or geriatric.

BRG1 and other SWI/SNF subunits have been implicated in human congenital heart diseases. Additionally, BRG1 is overexpressed in a subset of patients with hypertrophic cardiomyopathy, and this is associated with an αMHC-to-βMHC shift similar to what was observed in the Brg1 inducible mutant mouse model. Analogous to BRG1, HDAC and PARP activity increase during cardiac hypertrophy and heart failure (HF) in murine models and human patients, HDAC inhibitors as well as PARP inhibitors decrease cardiac hypertrophy, delay the progression from hypertensive cardiomyopathy to HF, and decrease cell death and HF after ischemic insults.

Despite our knowledge of SWI/SNF complexes in the developing heart and in cardiac disease, it is not clear what role, if any, they have in normal adult cardiac function. One challenge is that SWI/SNF complexes exhibit considerable subunit heterogeneity. However, BRG1 and BRM are the only catalytic subunits. Therefore, simultaneous ablation of Brg1 and Brm abrogates all DNA-dependent ATPase activity, impairs chromatin remodeling, and can be used to interrogate SWI/SNF function in toto regardless of subunit composition. We recently demonstrated that inducing a Brg1/Brm double mutation in vascular endothelial cells (VECs) in adult mice causes the spontaneous apoptosis of VECs, primarily in the heart, resulting in ischemia, cardiac dissections, heart failure, and death. Considering that this phenotype was not observed in either Brg1 or Brm single mutants, BRG1 and BRM are functionally redundant in adult VECs within the heart.

In the present invention, the role of BRG1 and BRM in the maintenance of adult cardiomyocytes is analyzed by inducibly deleting Brg1 on wild-type and Brm-deficient backgrounds. To our surprise, a severe cardiomyopathy that developed in double mutants minimally affected the morphology histologically but did have profound effects on conduction and mitochondrial dynamics including mitophagy. This phenotype culminated in sudden cardiac death after distinctive prolongation of PR intervals, QT interval, and widening of the QRS complex in the electrocardiogram (ECG). These results demonstrate for the first time the role of SWI/SNF in maintaining the coordinated transmission of the cardiomyocyte pacemaker in the intact heart.

The present inventors have discovered that the cardiomyocyte Brg1/Brm ATPases of the SWI/SNF complex are redundant and are vital to the maintenance of cardiac conduction in heart failure via regulation of cardiomyocyte chromatin remodeling. Specifically, cardiomyocyte Brg1 and Brm transcriptionally regulate ion channel and gap junction proteins involved in cardiac conduction. They show that inducible knockout of Brg1/Brm activity fails to maintain gap junction proteins (e.g., Cx40, Cx43) and ion channels (SCN5A), resulting in a progressive bundle brand block (cardiac conduction defect, the most common cause of death in heart failure). Brg1 and Brm's also have a role in maintaining mitochondrial protein quality control, by regulating and supporting mitochondrial fission, protein unfolding, and mitochondrial-specific autophagy, each of which are also implicated in heart failure. Since the pathophysiology of cardiac conduction defects similarly occurs via decreased gap junction proteins (e.g., Cx40, Cx43) and ion channels (e.g., SCN5A, KATP), the present inventors have identified that HDAC inhibitors and HAT activators may be useful for treating heart failure, heart attack, and related disorders including but not limited to cardiac conduction defect, arrhythmia, familial hypertrophic cardiomyopathy and/or long QT syndrome, and/or for increasing or improving conduction in the heart.

Accordingly, in one aspect, the invention relates to a method of increasing expression of Brg1 and/or Brm mRNA and/or BRG1 and/or BRM protein in a subject, comprising delivering to the subject an effective amount of a histone deacetylase (HDAC) inhibitor and/or a histone acetyltransferase (HAT) activator, thereby increasing expression of Brg1 and/or Brm mRNA and/or BRG1 and/or BRM protein observed in the subject as compared to a control. In some aspects, a control can be the level of expression of Brg1 and/or Brm mRNA and/or BRG1 and/or BRM protein observed in said subject prior to delivery of the effective amount of a histone deacetylase (HDAC) inhibitor and/or a histone acetyltransferase (HAT) activator. In some embodiments, an n estimate of the level of Brg1 (SMARCA4) can be about 14 FPKM (Fragments Per Kilobase Of Exon Per Million Fragments Mapped) (proteinatlas.org/ENSG00000127616-SMARCA4/tissue) and an n estimate for the level of Bmr (SMARCA2) can be about 41 FPKM (proteinatlas.org/ENSG00000080503-SMARCA2/tissue) (mRNA expression levels).

A further aspect of the invention relates to a method of decreasing expression of c-Myc mRNA and/or c-Myc protein in a subject, comprising delivering to the subject an effective amount of a histone deacetylase (HDAC) inhibitor and/or a histone acetyltransferase (HAT) activator, thereby decreasing expression of c-Myc mRNA and/or c-Myc protein in the subject as compared to a control. In some aspects, a control can be the level of expression of c-Myc mRNA and/or c-Myc protein observed in said subject prior to delivery of the effective amount of a histone deacetylase (HDAC) inhibitor and/or a histone acetyltransferase (HAT) activator.

Another aspect of the invention relates to a method of increasing expression of Cx40, Cx43, and/or Scn5a mRNA and/or Cx40, Cx43, and/or Scn5a protein in a subject, comprising delivering to the subject an effective amount of a histone deacetylase (HDAC) inhibitor and/or a histone acetyltransferase (HAT) activator, thereby increasing expression of Cx40, Cx43, and/or Scn5a mRNA and/or Cx40, Cx43, and/or Scn5a protein in the subject as compared to a control. In some aspects, a control can be the level of expression of Cx40, Cx43, and/or Scn5a mRNA and/or Cx40, Cx43, and/or Scn5a protein observed in said subject prior to delivery of the effective amount of a histone deacetylase (HDAC) inhibitor and/or a histone acetyltransferase (HAT) activator. In some embodiments, an n estimate of the level of Cx40 can be about 29 FPKM (proteinatlas.org/ENSG00000143140-GJA5/tissue); an n estimate of the level of Cx43 can be about 126 FPKM (proteinatlas.org/ENSG00000152661-GJA1/tissue); and an n estimate for the level of Scn5a can be about 32 FPKM (proteinatlas.org/ENSG00000183873-SCN5A/tissue).

An additional aspect of the invention relates to a method of increasing expression of Brg1, Brm, c-Myc, Cx40, Cx43, and/or Scn5a mRNA and/or BRG1, BRM Cx40, Cx43, and/or Scn5a protein in a subject, comprising delivering to the subject an effective amount of a histone deacetylase (HDAC) inhibitor and/or a histone acetyltransferase (HAT) activator, thereby increasing expression of Brg1, Brm, c-Myc, Cx40, Cx43, and/or Scn5a mRNA and/or BRG1, BRM, Cx40, Cx43, and/or Scn5a protein in the subject as compared to a control. In some aspects, a control can be the level of expression of Brg1, Brm, c-Myc, Cx40, Cx43, and/or Scn5a mRNA and/or BRG1, BRM, Cx40, Cx43, and/or Scn5a protein observed in said subject prior to delivery of the effective amount of a histone deacetylase (HDAC) inhibitor and/or a histone acetyltransferase (HAT) activator.

A further aspect of the invention relates to a method of increasing and/or improving conduction in the heart in a subject in need thereof. In some aspects, the invention provides a method of increasing and/or improving conduction in the heart in a subject in need thereof, comprising increasing the expression of Brg1 and/or Brm mRNA and/or BRG1 and/or BRM protein in the subject, thereby increasing and/or improving conduction in the heart in the subject as compared to a control. Another aspect of the invention provides a method of increasing and/or improving conduction in the heart in a subject in need thereof, comprising decreasing expression of c-Myc mRNA and/or c-Myc protein in the subject as compared to a control, thereby increasing and/or improving conduction in the heart in the subject. An additional aspect of the invention provides a method of increasing and/or improving conduction in the heart in a subject in need thereof, comprising increasing expression of Cx40, Cx43, and Scn5a mRNA and/or Cx40, Cx43, and/or Scn5a protein in the subject as compared to a control, thereby increasing and/or improving conduction in the heart in the subject. A further aspect of the invention provides a method of increasing and/or improving conduction in the heart in a subject in need thereof, comprising increasing expression of Brg1, Brm, Cx40, Cx43, and/or Scn5a mRNA and/or BRG1, BRM, Cx40, Cx43, and/or Scn5a protein and/or decreasing expression of c-Myc mRNA and/or c-Myc protein in the subject as compared to a control, thereby increasing and/or improving conduction in the heart in the subject. In some aspects, a control can be the level of expression of Brg1, Brm, c-Myc, Cx40, Cx43, and/or Scn5a mRNA and/or BRG1, BRM, Cx40, Cx43, and/or Scn5a protein in said subject prior to delivery of the effective amount of a histone deacetylase (HDAC) inhibitor and/or a histone acetyltransferase (HAT) activator.

Brg1/Brm deficiency defects in conduction can result in (1) 2^{nd}/3^{rd} degree Bundle Branch Block; (2) Slow heart rate; (3) Arrhythmia=coordination of the block pumping impaired (systolic and diastolic dysfunction), and (4) Sudden death. Accordingly, “improved conduction” as used herein refers to: (1) Faster recovery of depolarization (via conduction cells of the heart). The speed of the electrical conduction improves; (2) Faster Improved QT delay (ECG). ECG correction of long QT measurement; (3) Improving slow heart rate from conduction deficits, which would normalize heart rate (Curing BBB); and (4) Systolic and diastolic functional changes (ability of heart to move blood normalizes). As an example, improved cardiac conduction is discussed in Lu et al. J Cardiovasc Pharmacol. 60(1):88-99 (2012).

A still further aspect of the invention relates to a method of treating and/or preventing a cardiac conduction defect in a subject in need thereof. Thus, in one aspect, the invention provides a method of treating and/or preventing a cardiac conduction defect in a subject in need thereof, comprising increasing the expression of Brg1 and/or Brm mRNA and/or BRG1 and/or BRM protein in the subject, thereby treating and/or preventing the cardiac conduction defect in the subject. An additional aspect of the invention provides a method of treating and/or preventing a cardiac conduction defect in a subject in need thereof, comprising decreasing the expression of c-Myc mRNA and/or c-Myc protein in the subject, thereby treating and/or preventing the cardiac conduction defect in the subject. A further aspect of the invention provides a method of treating and/or preventing a cardiac conduction defect in a subject in need thereof, comprising increasing the expression of Cx40, Cx43, and/or Scn5a mRNA and/or Cx40, Cx43, and/or Scn5a protein in the subject, thereby treating and/or preventing the cardiac conduction defect in the subject. An additional aspect of the invention provides a method of treating and/or preventing a cardiac conduction defect in a subject in need thereof, comprising increasing expression of Brg1, Brm, Cx40, Cx43, and/or Scn5a mRNA and/or BRG1, BRM, Cx40, Cx43, and/or Scn5a protein and/or decreasing expression of c-Myc mRNA and/or protein in the subject, thereby treating and/or preventing a cardiac conduction defect in the subject. In some aspects, a control can be the level of expression of Brg1, Brm, c-Myc, Cx40, Cx43, and/or Scn5a mRNA and/or BRG1, BRM, Cx40, Cx43, and/or Scn5a protein observed in said subject prior to delivery of the effective amount of a histone deacetylase (HDAC) inhibitor and/or a histone acetyltransferase (HAT) activator.

A further aspect of the invention relates to a method of treating and/or preventing arrhythmia in a subject in need thereof. Thus, in one aspect the invention provides a method of treating and/or preventing arrhythmia in a subject in need thereof, comprising increasing the expression of Brg1 and/or Brm mRNA and/or BRG1 and/or BRM protein in the subject, thereby treating and/or preventing arrhythmia in the subject. A still further aspect of the invention provides a method of treating and/or preventing arrhythmia in a subject in need thereof, comprising decreasing the expression of c-Myc mRNA and/or c-Myc protein in the subject, thereby treating and/or preventing arrhythmia in the subject. Another aspect of the invention provides a method of treating and/or preventing arrhythmia in a subject in need thereof, comprising increasing the expression of Cx40, Cx43, and/or Scn5a mRNA and/or Cx40, Cx43, and/or Scn5a protein in the subject, thereby treating and/or preventing arrhythmia in the subject. A further aspect of the invention provides a method of treating and/or preventing arrhythmia in a subject in need thereof, comprising increasing expression of Brg1, Brm, Cx40, Cx43, and/or Scn5a mRNA and/or BRG1, BRM, Cx40, Cx43, and/or Scn5a protein and/or decreasing expression of c-Myc mRNA and/or c-Myc protein in the subject, thereby treating and/or preventing arrhythmia in the subject. In some aspects, a control can be the level of expression of Brg1, Brm, c-Myc, Cx40, Cx43, and/or Scn5a mRNA and/or BRG1, BRM, Cx40, Cx43, and/or Scn5a protein observed in said subject prior to delivery of the effective amount of a histone deacetylase (HDAC) inhibitor and/or a histone acetyltransferase (HAT) activator.

An additional aspect of the invention relates to a method of treating and/or preventing heart failure in a subject in need thereof. Accordingly, in one aspect the invention provides a method of treating and/or preventing heart failure in a subject in need thereof, comprising increasing the expression of Brg1 and/or Brm mRNA and/or BRG1 and/or BRM protein in the subject, thereby treating and/or preventing heart failure in the subject. A further aspect of the invention provides a method of treating and/or preventing heart failure in a subject in need thereof, comprising decreasing the expression of c-Myc mRNA and/or c-Myc protein in the subject, thereby treating and/or preventing heart failure in the subject. A still further aspect of the invention provides a method of treating and/or preventing heart failure in a subject in need thereof, comprising increasing the expression of Cx40, Cx43, and/or Scn5a mRNA and/or Cx40, Cx43, and/or Scn5a protein in the subject, thereby treating and/or preventing heart failure in the subject. Another aspect of the invention provides a method of treating and/or preventing heart failure in a subject in need thereof, comprising increasing expression of Brg1, Brm, Cx40, Cx43, and/or Scn5a mRNA and/or BRG1, BRM, Cx40, Cx43, and/or Scn5a protein and/or decreasing expression of c-Myc mRNA and/or c-Myc protein in the subject, thereby treating and/or preventing heart failure in the subject. In some aspects, a control can be the level of expression of Brg1, Brm, c-Myc, Cx40, Cx43, and/or Scn5a mRNA and/or BRG1, BRM Cx40, Cx43, and/or Scn5a protein observed in said subject prior to delivery of the effective amount of a histone deacetylase (HDAC) inhibitor and/or a histone acetyltransferase (HAT) activator.

An additional aspect of the invention relates to a method of treating and/or preventing familial hypertrophic cardiomyopathy in a subject in need thereof. Thus, in an aspect the invention provides a method of treating and/or preventing familial hypertrophic cardiomyopathy in a subject in need thereof, comprising increasing the expression of Brg1 and/or Brm mRNA and/or BRG1 and/or BRM protein in the subject, thereby treating and/or preventing familial hypertrophic cardiomyopathy in the subject. Another aspect of the invention provides a method of treating and/or preventing familial hypertrophic cardiomyopathy in a subject in need thereof, comprising decreasing the expression of c-Myc mRNA and/or c-Myc protein in the subject, thereby treating and/or preventing familial hypertrophic cardiomyopathy in the subject. A further aspect of the invention relates to a method of treating and/or preventing familial hypertrophic cardiomyopathy in a subject in need thereof, comprising increasing the expression of Cx40, Cx43, and/or Scn5a mRNA and/or Cx40, Cx43, and/or Scn5a protein in the subject, thereby treating and/or preventing familial hypertrophic cardiomyopathy in the subject. A still further aspect of the invention provides a method of treating and/or preventing familial hypertrophic cardiomyopathy in a subject in need thereof, comprising increasing expression of Brg1, Brm, Cx40, Cx43, and/or Scn5a mRNA and/or BRG1, BRM, Cx40, Cx43, and/or Scn5a protein and/or decreasing expression of c-Myc mRNA and/or c-Myc protein in the subject, thereby treating and/or preventing familial hypertrophic cardiomyopathy in the subject. In some aspects, a control can be the level of expression of Brg1, Brm, c-Myc, Cx40, Cx43, and/or Scn5a mRNA and/or BRG1, BRM, Cx40, Cx43, and/or Scn5a protein observed in said subject prior to delivery of the effective amount of a histone deacetylase (HDAC) inhibitor and/or a histone acetyltransferase (HAT) activator.

An additional aspect of the invention relates to a method of treating and/or preventing long QT syndrome (LQTS) in a subject in need thereof. Accordingly, in one aspect, the invention provides a method of treating and/or preventing long QT syndrome in a subject in need thereof, comprising increasing the expression of Brg1 and/or Brm mRNA and/or BRG1 and/or BRM protein in the subject, thereby treating and/or preventing long QT syndrome in the subject. Another aspect of the invention provides a method of treating and/or preventing long QT syndrome in a subject in need thereof, comprising decreasing the expression of c-Myc mRNA and/or c-Myc protein in the subject, thereby treating and/or preventing long QT syndrome in the subject. A further aspect of the invention provides a method of treating and/or preventing long QT syndrome in a subject in need thereof, comprising increasing the expression of Cx40, Cx43, and/or Scn5a mRNA and/or Cx40, Cx43, and/or Scn5a protein in the subject, thereby treating and/or preventing long QT syndrome in the subject. A still further aspect of the invention provides a method of treating and/or preventing long QT syndrome in a subject in need thereof, comprising increasing expression of Brg1, Brm, Cx40, Cx43, and/or Scn5a mRNA and/or BRG1, BRM Cx40, Cx43, and/or Scn5a protein and/or decreasing expression of c-Myc mRNA and/or c-Myc protein in the subject, thereby treating and/or preventing long QT syndrome in the subject. In some aspects, a control can be the level of expression of Brg1, Brm, c-Myc, Cx40, Cx43, and/or Scn5a mRNA and/or BRG1, BRM, Cx40, Cx43, and/or Scn5a protein observed in said subject prior to delivery of the effective amount of a histone deacetylase (HDAC) inhibitor and/or a histone acetyltransferase (HAT) activator.

In additional aspects, the methods of increasing and/or improving conduction in the heart, and/or treating and/or preventing a cardiac conduction defect, arrhythmia, heart failure, familial hypertrophic cardiomyopathy, and/or long QT syndrome in a subject in need thereof as described herein, can comprise consist essentially of and/or consist of delivering to the subject an effective amount of a histone deacetylase (HDAC) inhibitor and/or a histone acetyltransferase (HAT) activator.

As used herein, the terms “histone deacetylase” and “HDAC” are intended to refer to any member of a family of enzymes that remove acetyl groups from the α,ε-amino groups of lysine residues at the N-terminus of a histone. Unless otherwise indicated by context, the term “histone” is meant to refer to any histone protein, including H1, H2A, H2B, H3, H4, and H5, from any species. Histone deacetylases may include class I and class II enzymes, and may also be of human origin, including, but not limited to, HDAC-1, HDAC-2, HDAC-3, HDAC-4, HDAC-5, HDAC-6, HDAC-7, and HDAC-8.

As used herein, the terms “histone deacetylase inhibitor” and “HDAC inhibitor” are intended to refer to a compound which is capable of interacting with a histone deacetylase and inhibiting its enzymatic activity. The phrase “inhibiting histone deacetylase enzymatic activity” means reducing the ability of a histone deacetylase to remove an acetyl group from a histone. In some embodiments, such reduction of histone deacetylase activity is at least about 50%, at least about 75%, or at least about 90%. In other embodiments, histone deacetylase activity is reduced by at least about 95% or at least about 99%. In the methods of this invention, a single HDAC inhibitor and/or any combination of HDAC inhibitors may be employed.

Suitable HDAC inhibitors include, but are not limited to, short-chain fatty acid, a hydroxamic acid, a cyclic tetrapeptide, a benzamide, a tricyclic lactam, a sultam derivative, an organosulfur compound; an electrophilic ketone, pimeloylanilide o-aminoanilide (PAOA), depudecin, a psammaplin, Vorinostat, tubacin, curcumin, histacin, 6-Chloro-2,3,4,9-tetrahydro-1H-carbazole-l-carboxamide, CRA-024781, CRA-026440, CG1521, PXD101, G2M-777, CAY10398, CTPB MGCDO103, CUDC-100, and/or any derivative thereof, as well as any combination thereof.

In some aspects, a short-chain fatty acid can be, but is not limited to, butyrate, phenylbutyrate, pivaloyloxymethyl butyrate, N-Hydroxy-4-(3-methyl-2-phenyl-butyrylamino)-benzamide,4-(2,2-Dimethyl-4-phenylbutyrylamino)-N-hydroxybenzamide, valproate, valproic acid, and/or any derivative thereof, as well as any combination thereof.

In some aspects, a hydroxamic acid can be, but is not limited to, suberoylanilide hydroxamic acid (SAHA), oxamflatin, M-carboxycinnamic acid bishydroxamide, suberic bishydroxamate (SBHA), nicotinamide, scriptaid (SB-556629), scriptide, splitomicin, lunacin, ITF2357, A-161906, NVP-LAQ824, LBH589, pyroxamide, Panobinostat (LB589), givinostat (or gavinostat (originally ITF2357)), resminostat (RAS2410), CBHA, 3-C1-UCHA, SB-623, SB-624, SB-639, SK-7041, a propenamide, an aroyl pyrrolyl hydroxyamide, a trichostatin, and/or any derivative thereof.

In some aspects, a propenamide can be, but is not limited to, MC 1293 and/or any derivative thereof, a aroyl pyrrolyl hydroxyamide can be, but is not limited to, APHA Compound 8 and/or any derivative thereof, as well as any combination thereof, and a trichostatin can be, but is not limited to, trichostatin A, trichostatin C, and/or any derivative thereof, as well as any combination thereof.

In some aspects, a cyclic tetrapeptide can be, but is not limited to, a trapoxin, romidepsin, HC-toxin, chlamydocin, diheteropeptin, WF-3161, Cyl-1, Cyl-2, apicidin, depsipeptide (FK228), FR225497, FR901375, a spiruchostatin, a salinamide, a cyclic-hydroxamic-acid-containing peptide, and/or any derivative thereof, as well as any combination thereof.

In some aspects, a spiruchostatin can be, but is not limited to, spiruchostatin A, spiruchostatin B, spiruchostatin C, and/or any derivative thereof; and a salinamide can be, but is not limited to, salinamide A, salinamide B and/or any derivative thereof, as well as any combination thereof.

In some aspects, a benzamide can be, but is not limited to, M344, MS-275, CI-994 (N-acetyldinaline), tacedinaline, sirtinol, and/or any derivative thereof, as well as any combination thereof.

In some aspects, an organosulfur compound can be, but is not limited to, diallyl disulfide, sulforaphane; and/or any derivative thereof, as well as any combination thereof.

In some aspects, an electrophilic ketone can be, but is not limited to, α-ketoamide, trifluoromethylketone and/or any derivative thereof, as well as any combination thereof.

In some aspects, the HDAC inhibitor can be, for example, SAHA, tributyrin, romidepsin, belinostat, pracinostat Valproic acid, valproate, Panobinostat, Trichostatin A, Mocetinostat (MGCD0103), Abexinostat (PCI-24781), Entinostat (MS-275), SB939, Resminostat (4SC-201) an oral pan-HDACi, Givinostat, Quisinostat, Kevetrin, CUDC-101, AR-42, CHR-2845, CHR-3996, 4SC-202, CG200745, ACY-1215, ME-344, and/or Sulforaphane, as well as any combination thereof.

In some embodiments, a compound that can modulate Brg1 expression may include, but is not limited to, N-acetylcysteine (NAC) and/or adiponectin, as well as any combination thereof. (See, Ju et al. Journal of Diabetes Research Volume 2013 (2013), Article ID 716219, 8 pages (dx.doi.org/10.1155/2013/716219); Xu et al. The FASEB Journal. 2013; 27:1b618; and Li et al. Free Radic Biol Med. 2015 Mar. 17. pii: S0891-5849(15)00119-7. doi: 10.1016/j.freeradbiomed.2015.03.007).

Histone acetyltransferases (HAT) are enzymes that acetylate conserved lysine amino acids on histone proteins by transferring an acetyl group from acetyl CoA to form ε-N-acetyl lysine. Histone acetylation is generally linked to transcriptional activation and thus gene expression. Thus, histone acetyltransferases (HATs) can act as transcriptional coactivators. In some embodiments, a HAT activator can include, but is not limited to, benzamide, N-[4-chloro-3-(trifluoromethyl)phenyl]-2-ethoxy-6-pentadecyl (CTPB), TTK21, and/or nemorosome. In some embodiments, a HAT activator can be a BET bromodomain inhibitor, including, but not limited to, RVX 208, LY294002, and/or JQ1, as well as any combination thereof.

The HDAC inhibitor and/or HAT activator of this invention can be provided singly and/or in any combination and can also be provided as a composition (e.g., a pharmaceutical composition). In some embodiments, the composition can be present in a pharmaceutically acceptable carrier. The compositions described herein can be formulated for administration in a pharmaceutical carrier in accordance with known techniques. See, e.g., Remington, The Science And Practice of Pharmacy (latest edition). By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of this invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients.

A “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include, without limitation, any of the standard pharmaceutical carriers such as phosphate buffered saline solutions, water, emulsions such as oil/water emulsion, microemulsions and various types of wetting agents.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intrathecal, intraarterial, or intravenous), topical (i.e., both skin and mucosal surfaces, including airway surfaces) and transdermal administration, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for oral administration can be presented in discrete units, such as capsules, cachets, lozenges, or tables, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit\dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bis\tris buffer (pH 6) or ethanol/water.

An effective amount of a composition of this invention, the use of which is in the scope of present invention, will vary from composition to composition, and subject to subject, and will depend upon a variety of well known factors such as the age and condition of the patient and the form of the composition and route of delivery. An effective amount can be determined in accordance with routine pharmacological procedures known to those skilled in the art.

The frequency of administration of a composition of this invention can be as frequent as necessary to impart the desired therapeutic effect. For example, the composition can be administered one, two, three, four or more times per day, one, two, three, four or more times a week, one, two, three, four or more times a month, one, two, three or four times a year or as necessary to control the condition. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

In particular embodiments, the HDAC inhibitor and/or HAT activator is administered to the subject in a therapeutically effective amount, as that term is defined above. Dosages of pharmaceutically active compounds can be determined by methods known in the art, see, e.g., Remington, The Science And Practice of Pharmacy (21^{th} Ed. 2005). The therapeutically effective dosage of any specific compound will vary somewhat from compound to compound, and patient to patient, and will depend upon the condition of the patient and the route of delivery. In one embodiment, the compound is administered at a dose of about 0.01 to about 150 mg/kg body weight, e.g., about 0.01, 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150 mg/kg, and any range or value therein. In some instances, the dose can be even lower, e.g., as low as 0.001, 0.0005, or 0.0001 mg/kg or lower, and any range or value therein. In some instances, the dose can be even higher, e.g., as high as 200, 250, 300, 350, 400, 450, 500, 1000, 5000 mg/kg or higher, and any range or value therein.

The present invention is more particularly described in the Examples set forth below, which are not intended to be limiting of the embodiments of this invention.

### EXAMPLES

### Example 1

Mice. All mouse experiments were approved by the Institutional Animal Care and Use Committees (IACUC) review boards at the University of North Carolina at Chapel Hill and Case Western Reserve University and were performed in accordance with federal guidelines. The αMHC-Cre-ERT mice [also known as B6.Cg-Tg(Myh6-cre/Esr1)1JmkJ or αMHC-MerCreMer] were obtained from The Jackson Laboratory (#005657, Bar Harbor, Me.) and genotyped as previously described. The Brg1 conditional mutant mouse line and Brm constitutive mutant mouse line have been described. Genotyping of the Brg1 floxed and Δfloxed alleles and the Brm mutation was carried out by PCR.

To induce the Brg1 conditional mutation in adult cardiomyocytes, 3-6 month old male and female mice were provided rodent chow containing tamoxifen (Sigma-Aldrich #T5648, St. Louis, Mo.) over a 7-day period. The route of delivery and dose were selected to minimize a previously described artifact caused by high doses of tamoxifen in the presence of the αMHC-Cre-ERT transgene. Briefly, 500 mg of tamoxifen was mixed with 1 kg of ground-up rodent chow and then mixed with water, kneaded into pellets, and dried in a hood. Provided to mice ad libitum, the dose was estimated to be 80 mg/kg/day. After the 7-day treatment period, the tamoxifen-fortified chow was removed and replaced with the same chow lacking tamoxifen.

The bi-transgenic mouse line that inducibly overexpresses the human c-MYC cDNA in cardiomyocytes under the control of the αMHC promoter has been previously described. Mice were raised in the absence of doxycycline (Dox) to prevent developmental consequences from c-MYC overexpression. c-MYC was induced by feeding mice Dox-containing rodent chow (200 mg/kg, Bio-Serve, Frenchtown, N.J.) ad libitum. Dox had no effect on single transgenic littermates, which were used as controls in analyses performed in this study.

Echocardiography and ECGs. Conscious cardiac transthoracic echocardiography was performed on mice at the indicated time points using a VisualSonics Vevo 2100 ultrasound biomicroscopy system (VisualSonics, Inc., Toronto, Ontario, Canada) as previously described. Two-dimensional M-mode echocardiography was performed in the parasternal long-axis view at the level of the papillary muscle on loosely restrained mice. Anterior and posterior wall thickness was measured as distance from epicardial to endocardial leading edges. Left ventricular internal diameters were also measured. Left ventricular systolic function was assessed by ejection fraction (LV EF %=[(LV Vol; d-LV Vol; s/LV Vol; d)×100] and fractional shortening (% FS=[(LVEDD−LVESD)/LVEDD]×100). Investigators were blinded to mouse genotype from collection through waveform measurements. Each measurement represents the average of three cardiac cycles from each mouse.

Continuous electrocardiographies (ECGs) were monitored by surgically implanting a TA10ETA radiotelemetry device (Data Sciences International (DSI), St. Paul, Minn.) into the abdomen of mice anesthetized with isoflurane and transmitting the information to APR-1 receivers under the cages that were coupled to the Ponemah v.5.0 Physiology Platform for data analysis (DSI).

Histology and TEM. Histology was performed by fixing adult heart tissues in 4% paraformaldehyde or 10% formalin, embedding in paraffin, and cutting 5-μm sections according to standard procedures. Sections were either stained with hematoxylin and eosin (H&E), or Mason's Trichrome, or processed for immunohistochemistry (IHC) using a BRG1 rabbit polyclonal antibody (Millipore #07-478, Temecula, Calif.) or a GRP78 mouse monoclonal antibody. Imaging of stained sections was obtained using Aperio Scanscope and Aperio Imagescope software (version 10.0.36.1805, Aperio Technologies, Inc., Vista, Calif.).

For transmission electron microscopy (TEM), hearts were fixed in 2% parformaldehyde and 2.5% glutaraldehyde in 0.15 M sodium phosphate buffer (pH 7.4), overnight and then post-fixed with 1% osmium tetroxide/0.15M sodium phosphate buffer. Samples were dehydrated with increasing concentrations of ethanol, infiltrated and embedded in Polybed 812 epoxy resin (Polysciences, Warrington, Pa.). One-micron sections were prepared to select representative areas by light microscopy, and 70-nm ultrathin sections were cut with a diamond knife. Sections were mounted on 200 mesh copper grids and staining with 4% aqueous uranyl acetate and Reynolds' lead citrate. Sections were observed with a LEO EM910 transmission electron microscope operating at 80 kV (LEO Electron Microscopy, Thornwood, N.Y.) and photographed with a Gatan Orius SC1000 CCD Digital Camera and Digital Micrograph 3.11.0 (Gatan, Pleasanton, Calif.). Mitochondria cross-sectional areas were measured using NIH ImageJ at magnifications of 5,000-10,000×. The global scale was set according to the image specific scale generated by Gatan camera output. An average of 2,500 mitochondria were analyzed from 30 fields from multiple levels from three hearts per mouse cohort.

Autophagic Flux. Autophagic flux was determined in vivo from the titration of bafilomycin A1 as previously described. Briefly, stock bafilomycin A1 (BFA, Santa Cruz Biotechnology #201550A, Santa Cruz, Calif.; 3 mM stock in dimethyl sulfoxide) diluted in phosphate-buffered saline (40% dimethyl sulfoxide final) was injected intraperitoneally to a final concentration of 3 mmol/kg in 200 μL and repeated one hour later. One hour after the second injection, mice were euthanized and heart tissues were collected for analysis by Western immunoblot for LC3B isoforms (Sigma-Aldrich #L7543). The Beclin autophagy marker was also analyzed by Western immunoblot (Cell Signaling #3495P, Beverly, Mass.).

Protein Aggregation Assay. Protein aggregation was assessed using an Enzo Proteostat Protein Aggregation Assay (#ENZ-51023, Farmingdale, N.Y.) in conjunction with their pre-formulated aggregation standards (#ENZ-51039). Briefly, protein lysates were prepared from flash-frozen apical cardiac tissues using 8M urea lysis buffer, protein concentrations were determined by Bradford assays, and assays were performed in 96-well, black-walled, clear-bottom plates read on a Clariostar microplate reader at 550 nm excitation and 600 nm emission with gain setting between 1000-2000. Pre-amyloid oligomer staining was performed.

RNA Isolation. Cardiac tissues were homogenized using a TissueLyser LT (Qiagen N.V. #69980, Venlo, The Netherlands) according to the manufacturer's protocols. Approximately 20-40 mg of apical ventricle was homogenized in 1 mL of Trizol (Life Technologies #15596-026, Carlsbad, Calif.) using a 5-mm stainless steel bead (Qiagen N.V. #69989). Chloroform (200 up was added, centrifuged at 12,000 g (15 min at 4° C.), isopropanol (0.5 mL) was then added to the aqueous phase, centrifuged at 12,000 g (10 min at 4° C.) and the resulting RNA pellet was washed with 1 mL of 75% ethanol, centrifuged at 7500 g (5 min at 4° C.). The resulting pellet was dried and resuspended in RNase-free water. RNA concentrations were then determined by UV spectroscopy (absorbance of 260-280 nm).

Real-Time PCR. RNAs (500 ng) were reverse-transcribed using iScript reverse transcription supermix (Bio-Rad Laboratories #170-8841, Hercules, Calif.). TaqMan gene expression assays (Life Technologies) were performed using universal TaqMan master mix (Life Technologies #4304437) to measure markers of hypertrophy and fission/fusion. Cardiac hypertrophy fetal gene expression was monitored using probes for β-MHC (Mm00600555_m1), skeletal muscle α-actin (Mm00808218_g1), ANF (Mm01255747_g1), and BNP (Mm00435304_g1) mRNAs. Fission and fusion gene expression was quantified using probes for Opa1 (Mm00453879_m1), Drp1 (Mm01342903_m1), Fis1 (Mm00481580_m1), and Mfn1 (Mm00612599_m1) mRNAs as described previously.

Mitochondrial number was quantified by qPCR on DNA prepared from whole-heart homogenates using the DNAeasy Blood and Tissue Kit (Qiagen #69506). SYBR Green and primers for the mitochondrial DNA included mtNd1, mtCO1, and mtCytb1; H19 was run in parallel as a nuclear DNA control as previously detailed. Five housekeeping genes were analyzed including Gapdh (NM_001289726.1, UPL probe #80 #689038, primers: tgtccgtcgtggatctgac (SEQ ID NO:1), cctgcttcaccaccttcttg (SEQ ID NO:2)), Atcb (NM_007393.3, UPL probe #64 #688635, primers: ctaaggccaaccgtgaaaag (SEQ ID NO:3), accagaggcatacagggaca (SEQ ID NO:4)), Pgk (NM_008828.2, UPL probe #108 #692276, primers: tacctgctggctggatgg (SEQ ID NO:5), cacagcctcggcatatttct (SEQ ID NO:6)), and G6PHD (NM_008062.2, UPL probe #76, #688996, primers: ttaaatgggccagcgaag (SEQ ID NO:7), tgctctgccatgatgttttc (SEQ ID NO:8)) using the Roche universal probe system).

Primer pairs for other RT-qPCR assays:

(SEQ ID NO: 15)
Atf6 Forward: TAC GTT GTC TCA TTT CGA AGG
(SEQ ID NO: 16)
Reverse: CAT TTT TGG TCT TGT GGT CTT G
(SEQ ID NO: 17)
Probe: FCA TCT GCT TAT TAC CAG CTA CCA CCC AQ
(SEQ ID NO: 18)
Atf3 Forward: GCC TGC CGA AAG AGT CAG AG
(SEQ ID NO: 19)
Reverse: TCT CAT TCT TCA GCT CCT CAA
(SEQ ID NO: 20)
Probe: FTG GGC CTT CAG CTC AGC ATT CAC ACQ
(SEQ ID NO: 21)
Ire1a Forward: TCC GAG CCA TGA GAA ACA AG
(SEQ ID NO: 22)
Reverse: GAG CCC AGC GTC TCC TGA A
(SEQ ID NO: 23)
Probe: FAC CAC TAC CGG GAG CTC CCC GTG Q
Xbp-1 Full, Spliced
(SEQ ID NO: 24)
Common Forward: GTT CCA GAG GTG GAG GCC A
(SEQ ID NO: 25)
Full Reverse: TAG TCT GAG TGC TGC GGA CT
(SEQ ID NO: 26)
Spliced Reverse: GCC TGC ACC TGA CTC AGC AG
(SEQ ID NO: 27)
Common Probe: FAC CCG GCC ACC AGC CTT ACT CCA Q
(SEQ ID NO: 28)
Chop Forward: CGA AGA GGA AGA ATC AAA AAC C
(SEQ ID NO: 29)
Reverse: TGT GAC CTC TGT TGG CCC T
(SEQ ID NO: 30)
Probe: FAC TAC TCT TGA CCC TGC GTC CCT AGQ
(SEQ ID NO: 31)
Atf4 Forward: CCT CGG AAT GGC CGG CTA T
(SEQ ID NO: 32)
Reverse: GGA AAA GGC ATC CTC CTT G
(SEQ ID NO: 33)
Probe: FAT GAT GGC TTG GCC AGT GCC TCA GQ
(SEQ ID NO: 34)
Grp78 Forward: ATA AAC CCC GAT GAG GCT GT
(SEQ ID NO: 35)
Reverse: ACC AGA TCA CCT GTA TCC TG
(SEQ ID NO: 36)
Probe: FCT ATG GTG CCG CTG TCC AGG CTG Q
(SEQ ID NO: 37)
PPARgl: Forward: CTG ACG GGT TCT CGG TTG A
(SEQ ID NO: 38)
Reverse: ATC AGT GGT TCA CCG CTT CT
(SEQ ID NO: 39)
Probe: FCT GAG AAG TCA CGT TCT GAC AGG ACQ
(SEQ ID NO: 40)
Fabp4 Forward: CAC CGA GAT TTC CTT CAA ACT
(SEQ ID NO: 41)
Reverse: GCC ATC TAG GGT TAT GAT GC
(SEQ ID NO: 42)
Probe: FCG TGG AAT TCG ATG AAA TCA CGC GCA Q

BestKeeper Analysis. For gene expression calculations, the threshold value at Cross point (Cp) was determined for each target when the number of cycles at which the fluorescence rose appreciably above the background fluorescence. Relative target gene expression levels were first normalized to BestKeeper Index and then normalized to control groups. Fold change values (calculated by the formula 2 (−ΔΔCq)) were used in the analysis. To select the suitable reference genes in qPCR for normalization, the stability of the candidate gene expression was statistically analyzed with a Microsoft Excel-based software: BestKeeper applet. BestKeeper estimated the gene expression variation for reference by the calculated standard deviation (SD) and coefficient of variation (CV) using their raw Cp values derived from qPCR as input. The candidate genes with SD values lower than 1 were considered to be stably expressed. The most stably expressed genes were determined based on the Pearson correlation coefficient (r) and the BestKeeper index (BI), which were the geometric means of Cp values of these candidate reference genes, and the corresponding p values. The Bestkeeper applet was used to calculate the gene expression variation of reference genes. This program was designed based on the input from each reference's Cp values. Reference gene fitness was determined by measuring a panel of the following genes: Gapdh, 18S, β-actin, Pgk1 and P6PDH. Based on the statistical data for maximal stability analysis, three reference genes, Gapdh, 18S and Pgk1, were selected. The descriptive statistical SD, CV, correlation coefficient (r) and corresponding P values of the 3 selected reference genes are shown in Table 1.

RNA-seq Transcriptomics. Total RNAs were extracted (Norgen #25700, Thorold, Ontario, Canada), and their integrities were confirmed using a BioAnalyzer (Agilent, Santa Clara, Calif.). A PolyA+ isolation kit (Promega #Z5300, Madison, Wis.) was used to enrich for mRNAs, and strand-specific RNA-seq libraries were prepared using a kit (Clontech #634836, Mountain View, Calif.). A QC step was performed using a Quibit 2.0 fluorometer (Life Technologies). Libraries were pooled at 2-nm concentrations, and the samples were then subject to cBot cluster generation using TruSeq Rapid PE Cluster Kit (Illumina, San Diego, Calif.). The amplified libraries were sequenced at the High-Throughput Sequencing Facility at UNC using a HiSeq2500 instrument (Illumina) 50M paired-end reads were analyzed. RNA-seq data were aligned with MapSplice, and genes were quantified with RSEM. Gene expression estimates were upper quartile normalized.

Differential expression was assessed between samples by calculating a t-statistic for each gene. P-values from the t-tests were transformed to q-values to control for multiple hypothesis testing and evaluate the false discovery rate (FDR) of the resulting candidates. A q-value threshold of 0.05 was applied to identify differentially expressed genes. Differential expression was assessed between samples by SAM using the two-class unpaired option. A FDR threshold of 0.05 was applied to identify differentially expressed genes. For heat maps, cluster analysis of candidate genes was performed using average linkage hierarchical clustering and Pearson correlation.

ChIP Assays. ChIP assays were performed as previously described. Briefly, 30 mg of cardiac tissues were pulverized in liquid nitrogen using a mortar and pestle and then crosslinked in 1% formaldehyde at room temperature for 10 minutes. After the crosslinking reaction was stopped with 0.125M glycine, the tissues were lysed, and the chromatin was sonicated into 200-500-bp fragments. 5% of the sonicated chromatin was removed as input, and the remainder of each sample was immunoprecipitated overnight at 4° C. using a BRG1 antibody (J1). Duplicate samples were immunoprecipitated with rabbit IgG as a negative control Immunoprecipitants were pulled down using protein A/G beads (Santa Cruz Biotechnology), washed following standard procedures, and eluted in 25 μL of ddH₂O.

qPCR was performed using Power SYBR Green Master Mix (Life Technologies) using the following primer pairs. Cx40: Forward, CTTTCTCGACTGGTGAGGAA (SEQ ID NO:9); Reverse, GAGCCTGTTAGTTGCTCCCG (SEQ ID NO:10) (450 nM final concentration of each). Cx43: Forward, CCCTTCTCGTCAGCACATTG (SEQ ID NO:11); Reverse, AGCCACTGACTCAACTGGAA (SEQ ID NO:12) (300 nM final concentration of each). Scn5a: Forward, GTCAGAGTGGTGGGCTG (SEQ ID NO:13); Reverse, GATCCCCACATCCCACGG (SEQ ID NO:14) (250 nM final concentration of each). The cycling parameters consisted of: 95° C. for 45 sec., 55° C. for 60 sec., and 72° C. for 60 sec. Dissociation curves and agarose gels demonstrated a single PCR product in each case without primer dimers. Relative enrichment was determined from a standard curve of serial dilutions of input samples.

Statistics. SigmaPlot (Systat Software, Inc., San Jose, Calif.) was used to determine significant statistical difference by One-way ANOVA followed by post-hoc analysis using the Holm-Sidak method or a Student's t-test. A p value<0.05 was considered significant.

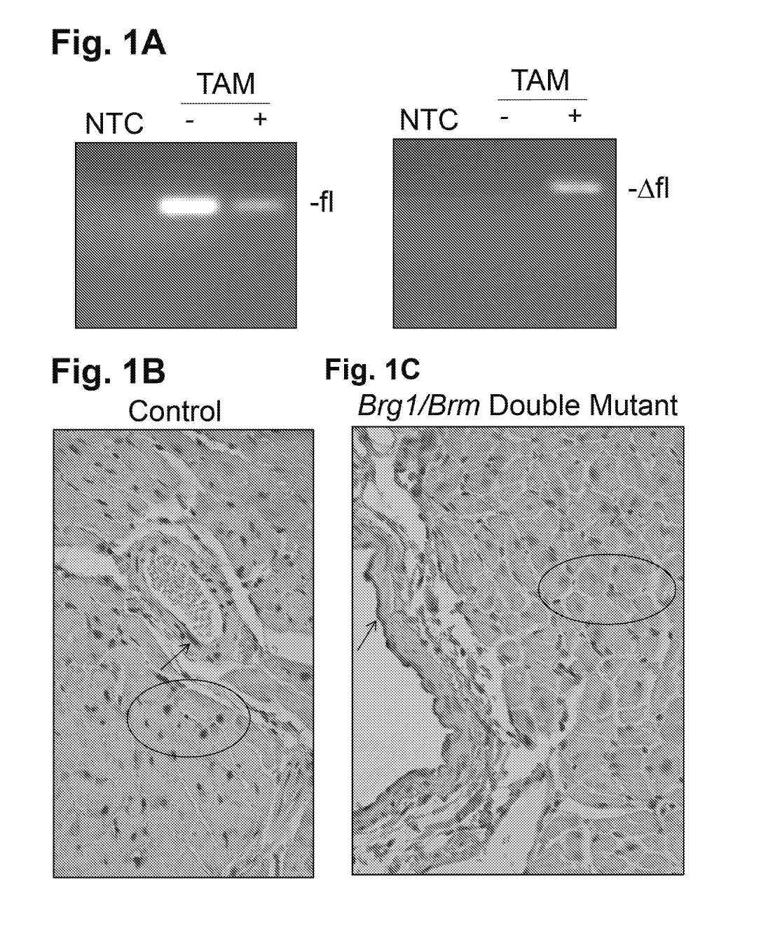

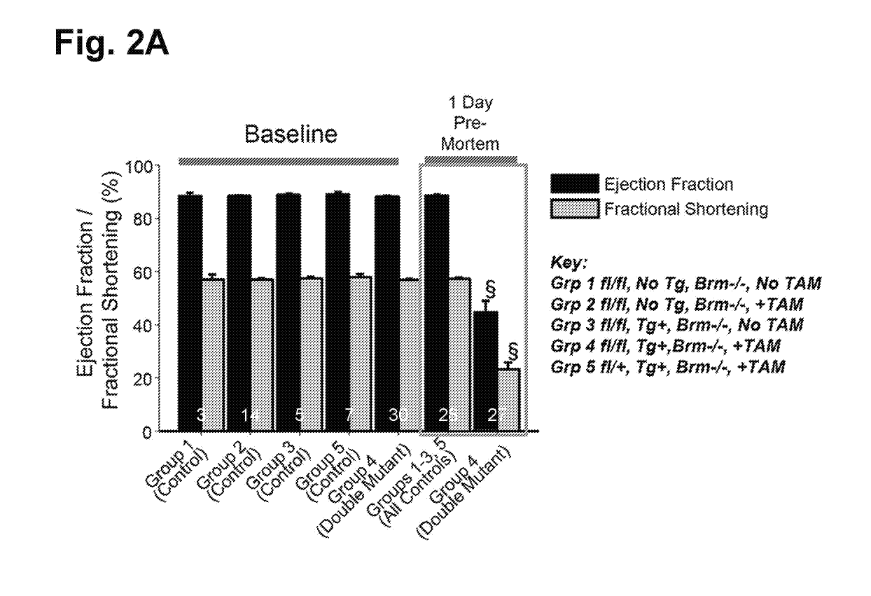

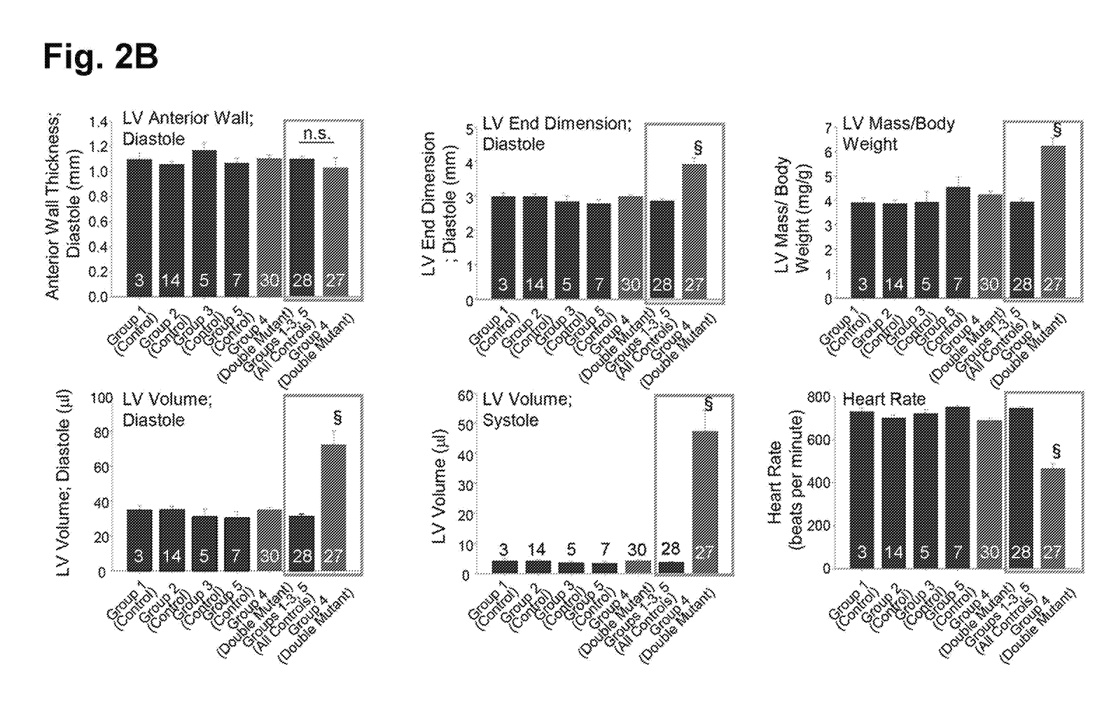

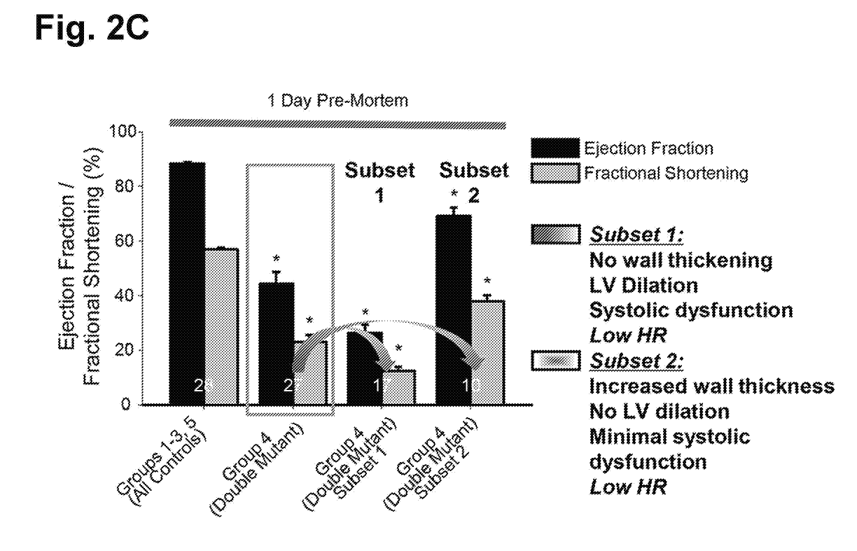

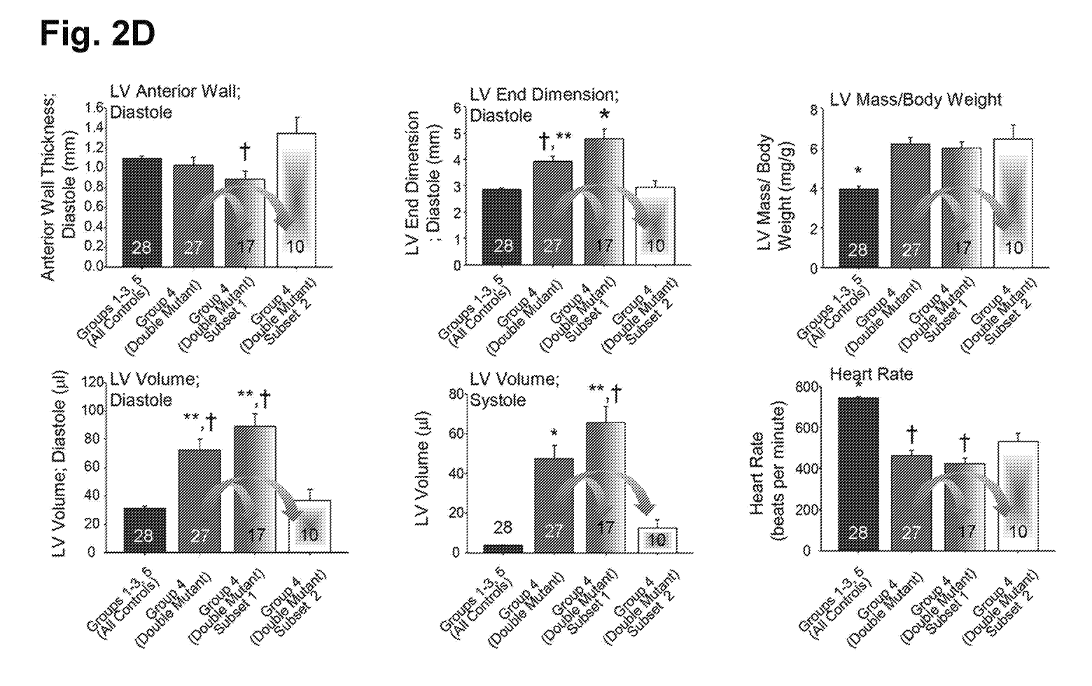

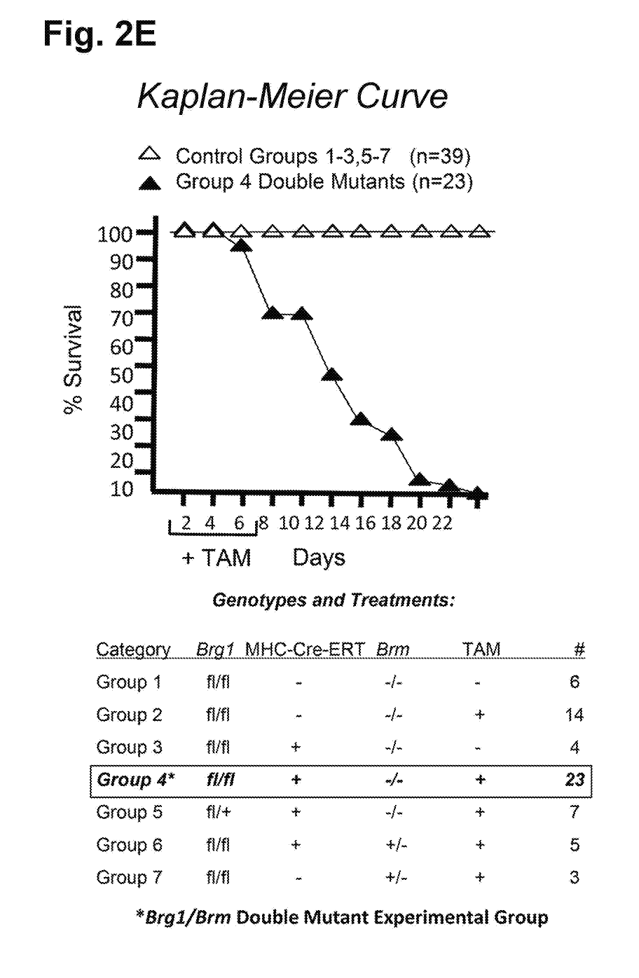

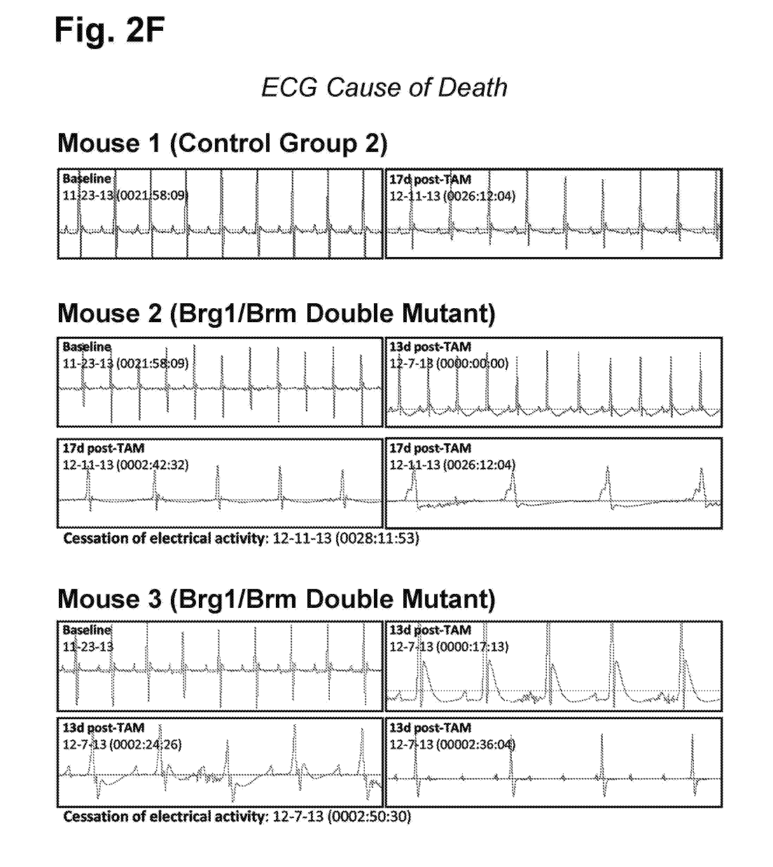

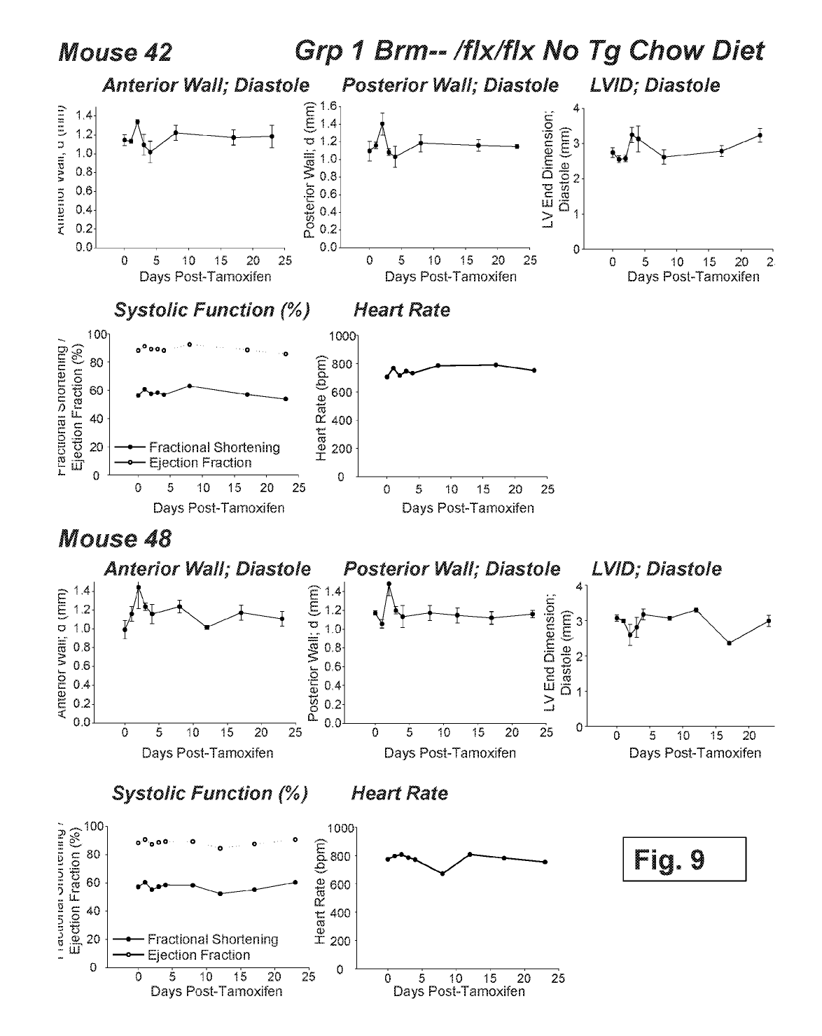

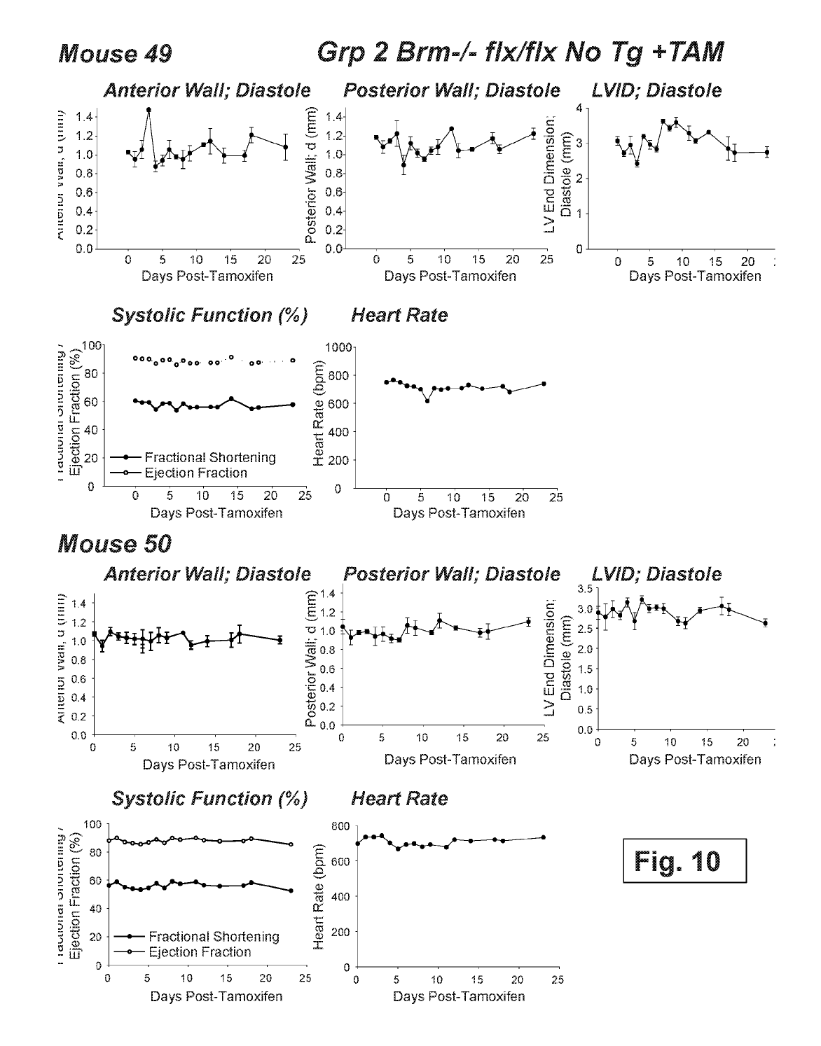

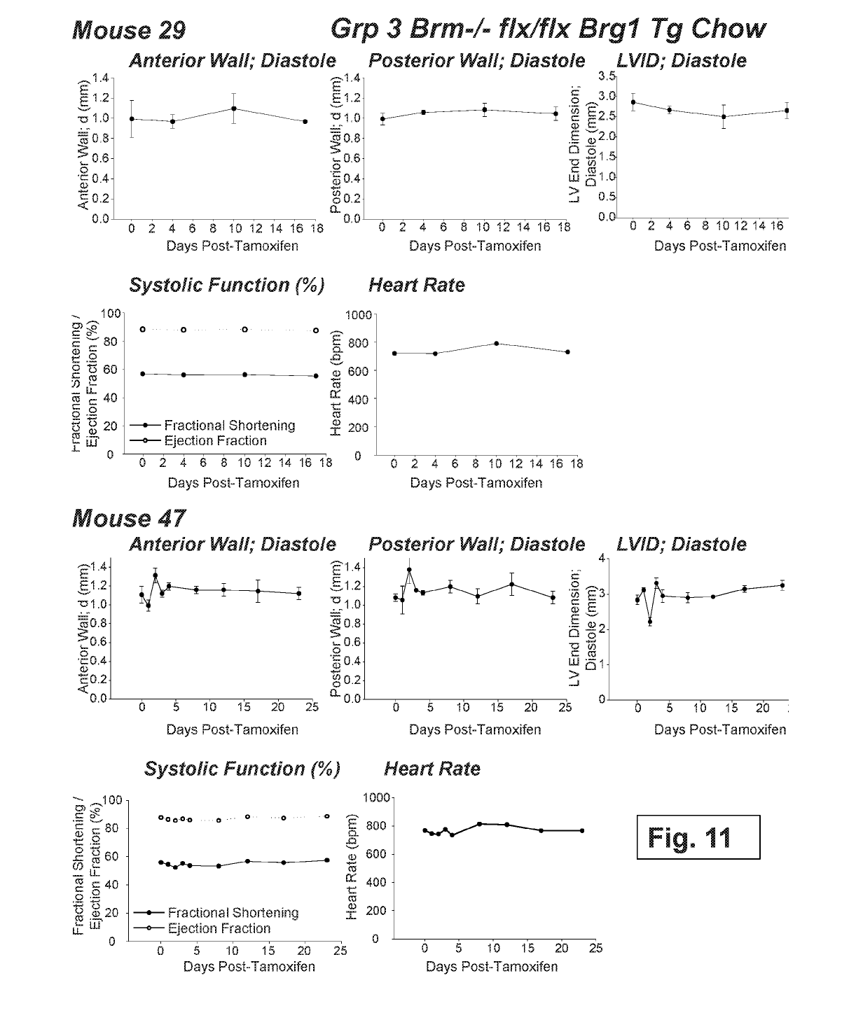

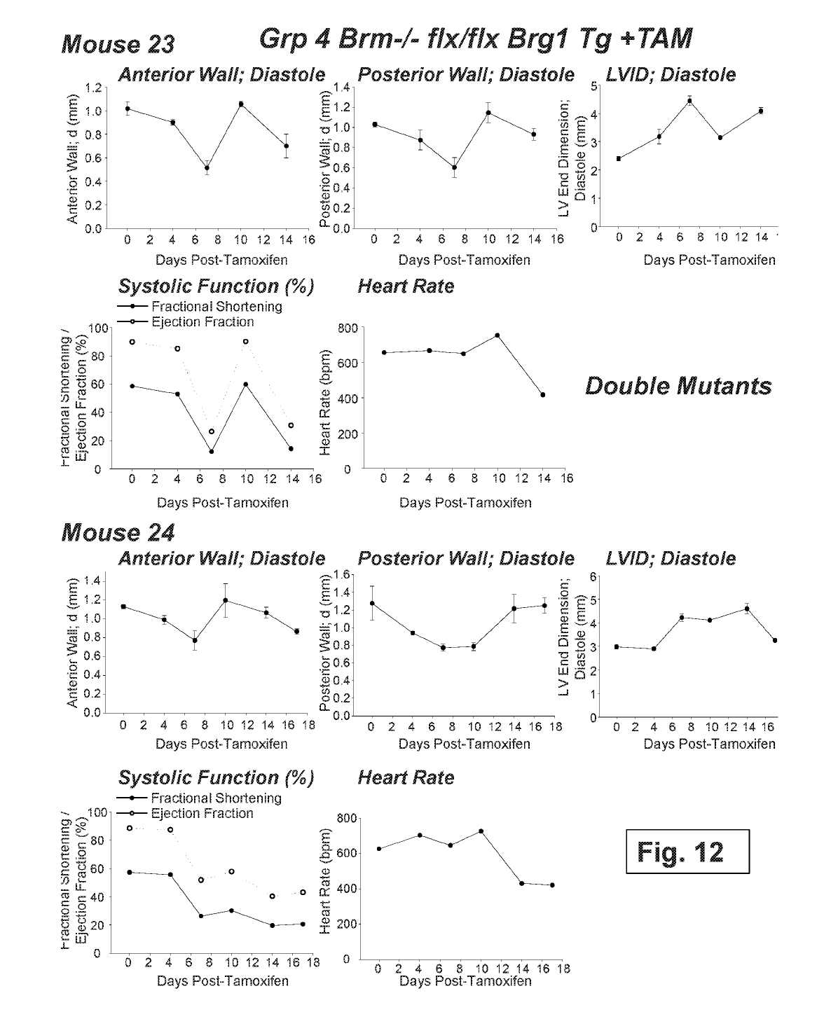

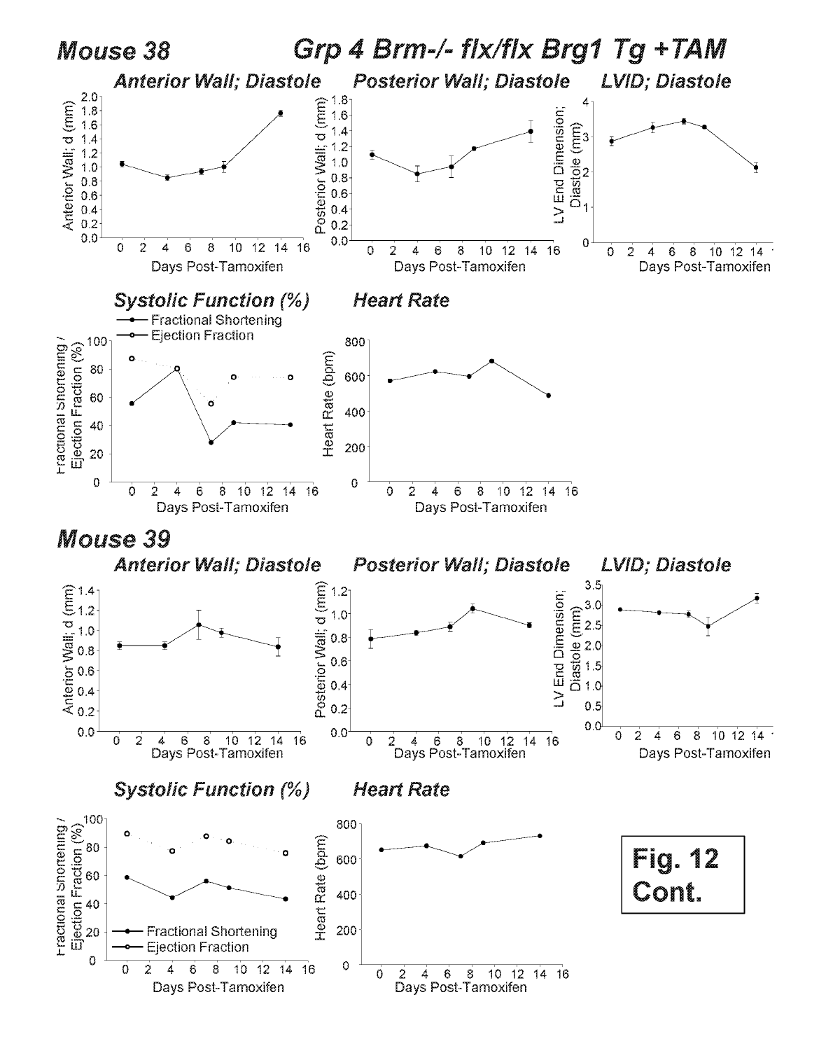

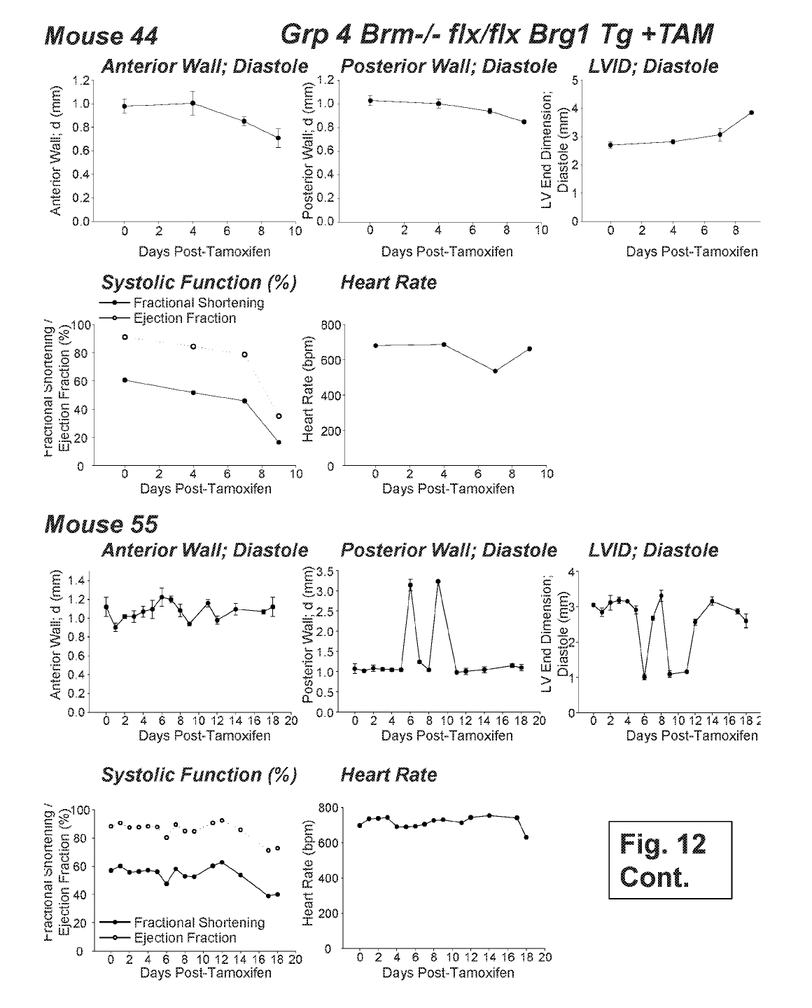

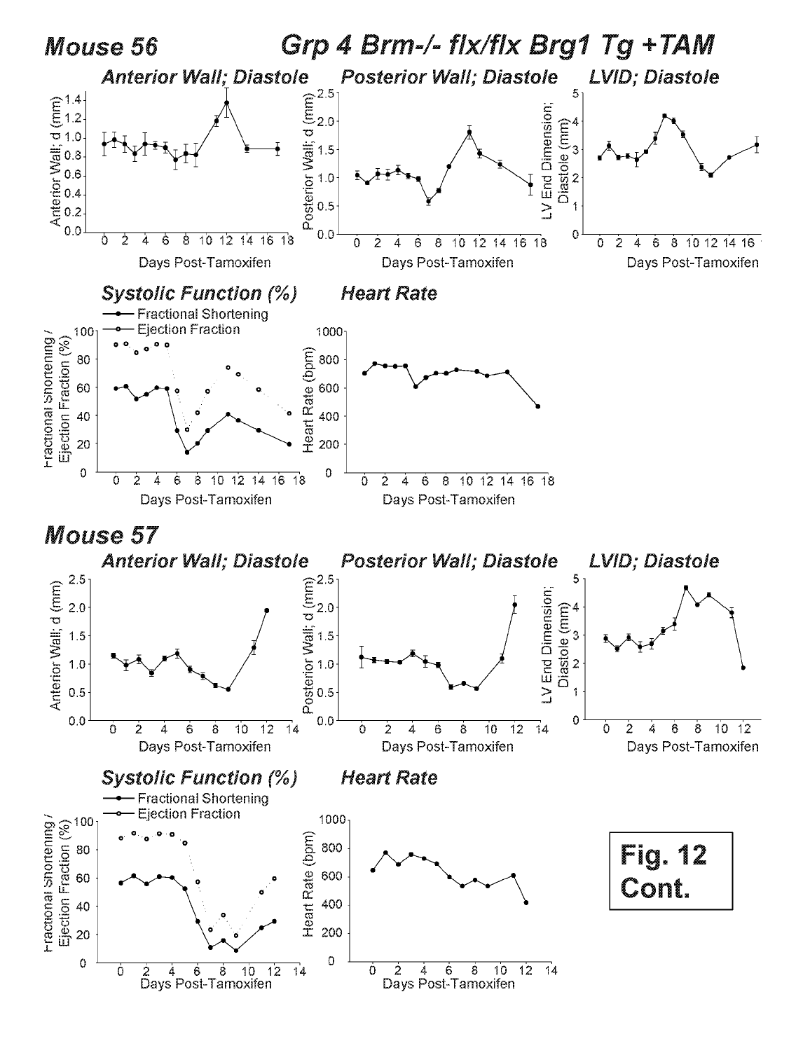

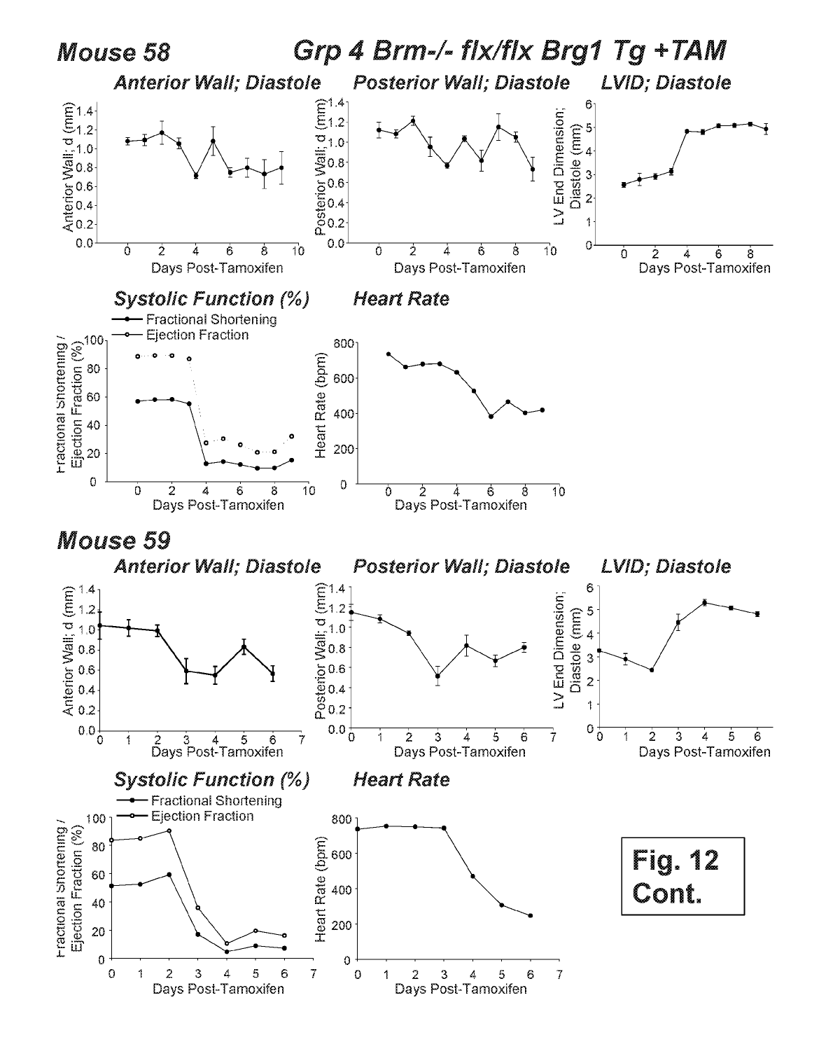

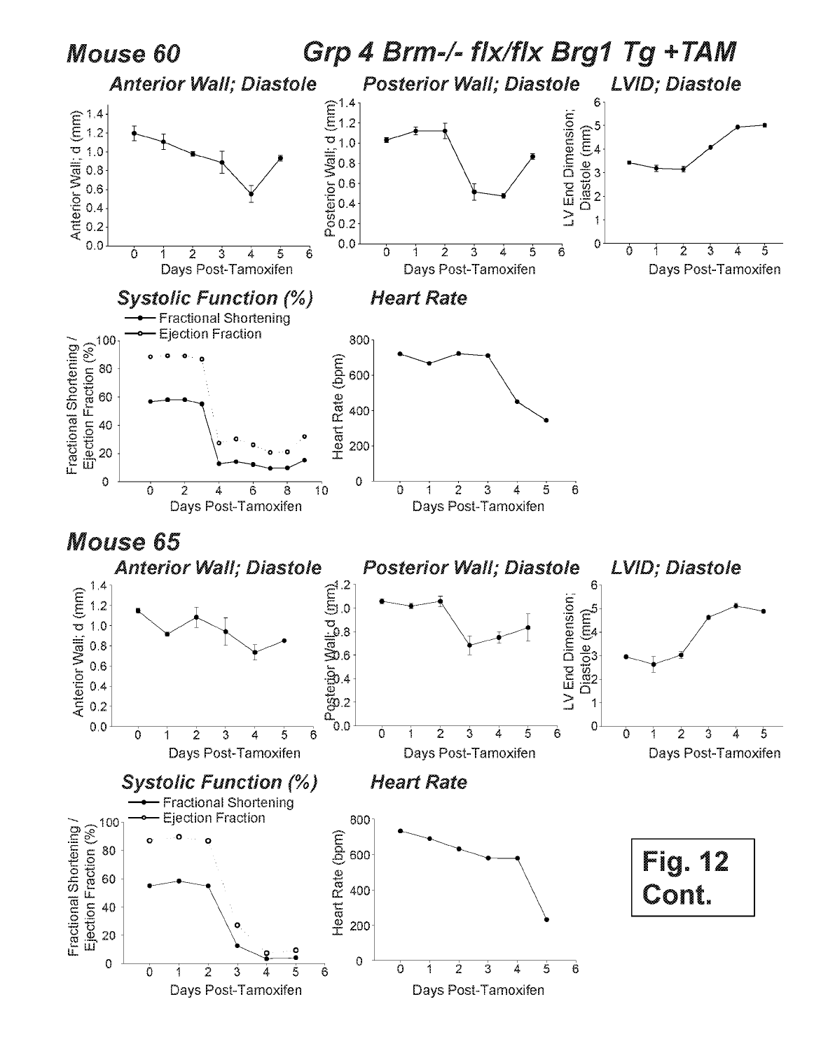

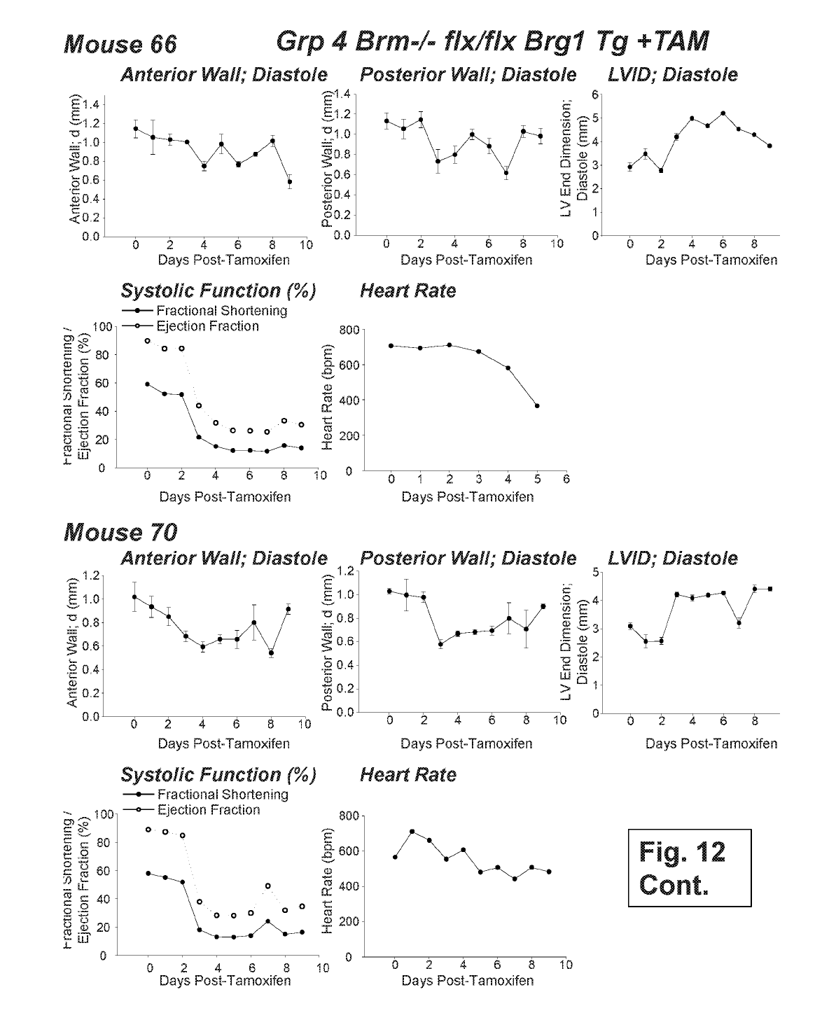

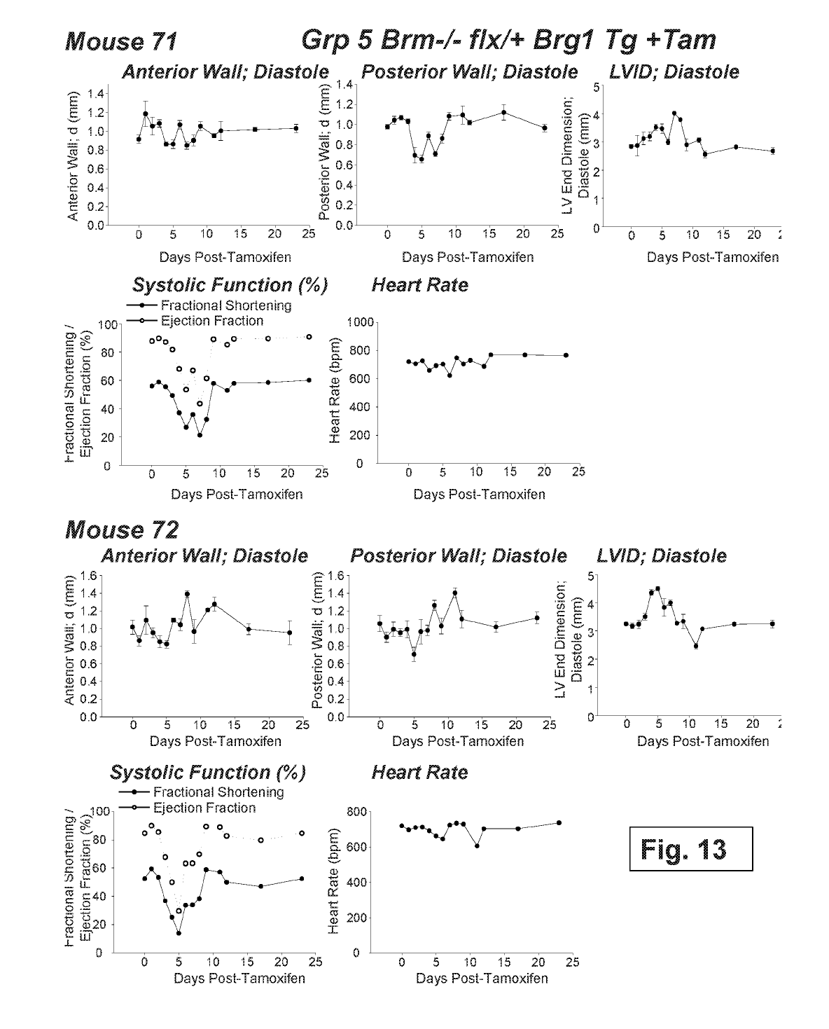

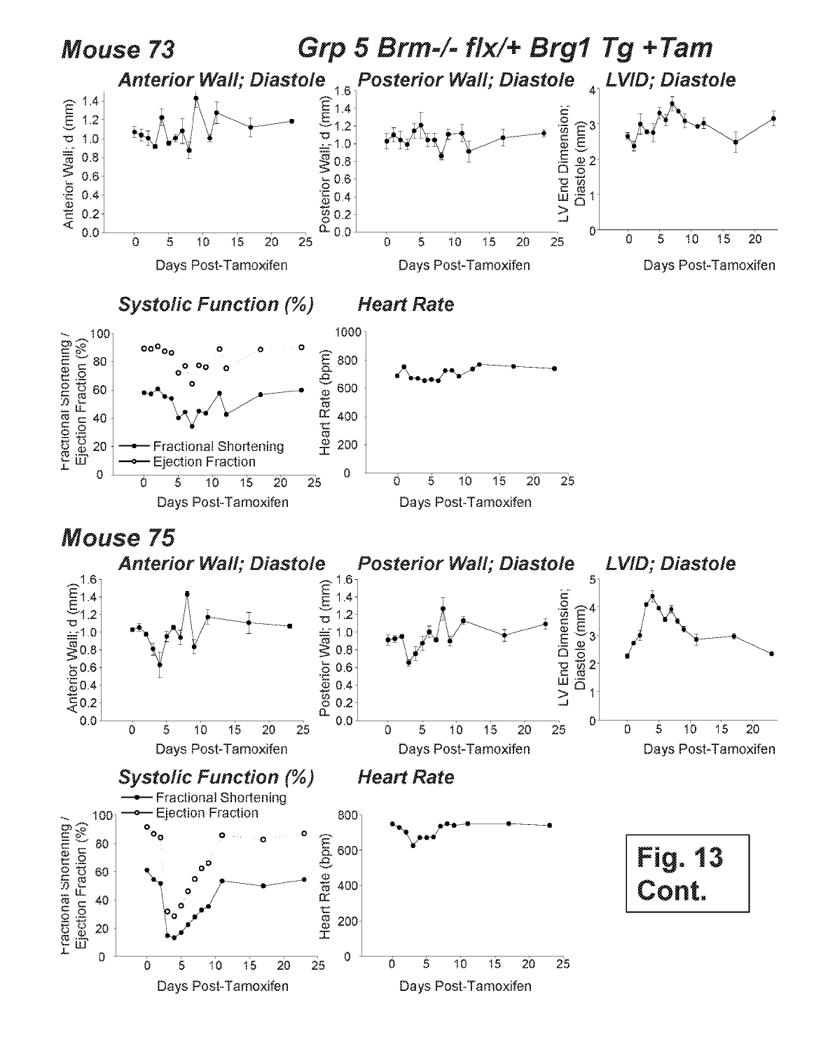

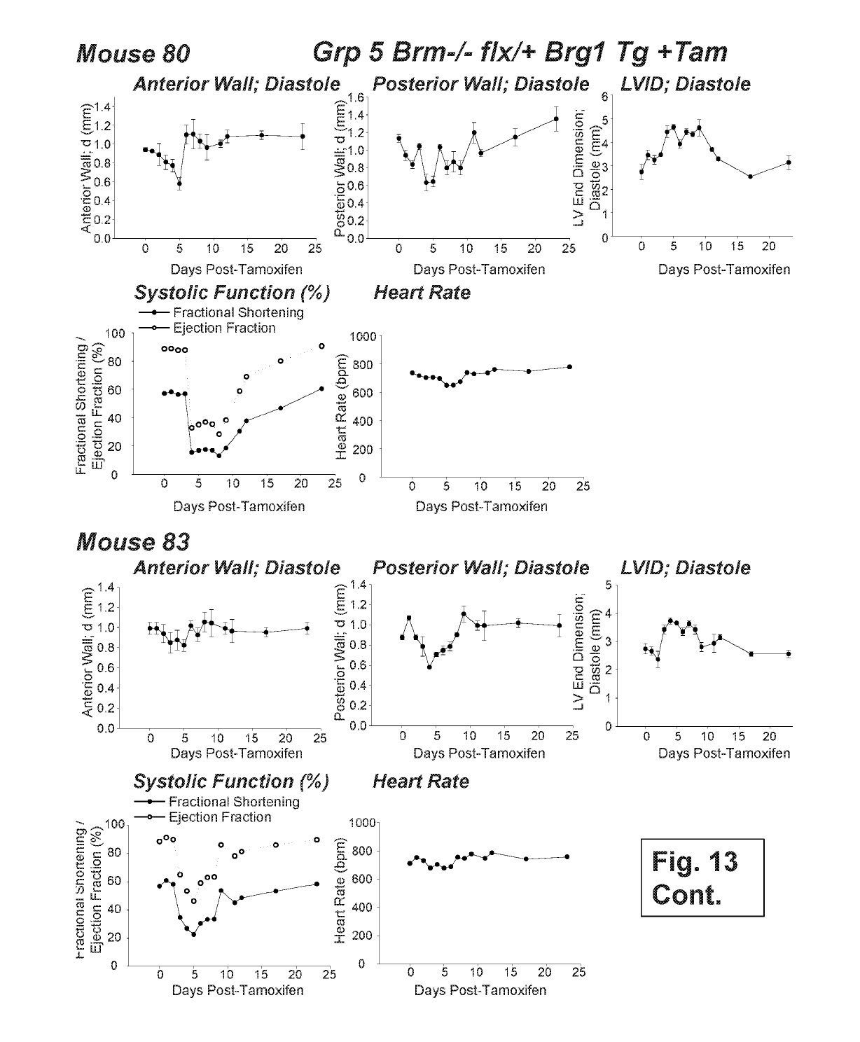

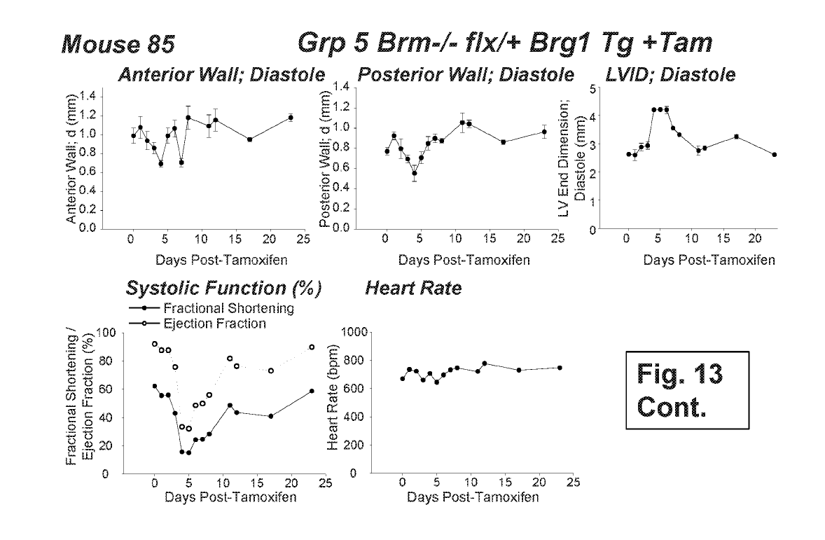

A Novel Mouse Model Reveals SWI/SNF Complexes are Essential in Adult Cardiomyoctes. To investigate the combined role of BRG1 and BRM in adult cardiomyocytes, we crossed mice carrying an inducible, cardiomyocyte-specific αMHC-Cre-ERT transgene to Brg1^{fl/fl}; Brm^{-/-} mice. Adult Brg1^{fl/fl}; αMHC-Cre-ERT^{+/0}; Brm^{-/-} mice were then fed tamoxifen-fortified chow for 7 days to induce the Brg1 floxed-to-Δfloxed excision event, which was confirmed by both PCR and IHC (FIG. 1). These mice (herein referred to as Brg1/Brm double mutants) were monitored by echocardiography until they died, which occurred within 22 days of initiating the tamoxifen diet (FIG. 2, FIGS. 9-13). Given the cardiomyocyte specificity of the Brg1 conditional mutation, it is not surprising that Brg1/Brm double mutants developed severe left ventricular (LV) systolic dysfunction as evidenced by decreased ejection fraction % and decreased fractional shortening % as well as LV dilation as evidenced by a widening LV that contracted less (FIG. 2A, Table 2).

Six groups of control mice were analyzed in parallel (listed in FIGS. 2A and 2E, FIGS. 9-13) to ensure that we were observing a Brg1/Brm double-mutant phenotype rather than an artifact such as tamoxifen itself or tamoxifen-induced Cre dysfunction, which has been previously described. A mild tamoxifen-induced Cre effect on cardiac function was observed briefly in Group 5 controls (FIG. 9), but it was a transient event that returned to baseline function in each mouse and did not result in any mortality. The control groups also demonstrated that the phenotype was not observed in either Brg1 or Brm single mutants and was therefore specific to Brg1/Brm double mutants (FIGS. 2A and 2E).

Brg1/Brm Double Mutants Exhibit a Cardiac Conduction Phenotype and Lethal Atrioventricular Block. The Brg1/Brm double-mutant mice experienced a rapid and progressive decline in cardiac function (FIGS. 2A-B, FIG. 9), resulting in mortality at mean day 11.6±1.5, range 6-22 days relative to the first day of tamoxifen treatment (FIG. 2E). Interestingly, a characteristic bradycardia was identified in the 24 hours before the mice died (herein referred to as 1-day pre-mortem) (FIG. 2B, Table 2), which could be characterized by two cardiac phenotypes: 1) a dilated cardiomyopathy with severe dysfunction and significantly thinner walls (EF %<50%, mean 26.4±3.1%); and 2) a hypertrophic cardiomyopathy with less severe systolic dysfunction (EF %>50%, mean 69.1%) (FIGS. 2C and 2D, Table 3). Both phenotypes had significantly decreased heart rates (422±28 and 532±40, respectively) by conscious echocardiography (FIG. 2D). These findings suggested a defect in the conduction system, either at the level of the cardiomyocyte ion conduction channels or by the specialized cardiomyocyte-derived conduction system in the heart.

To investigate this further and determine the cause of death, continuous ECG telemetry was performed (FIG. 2F). Mice were implanted with telemetry units, allowed to recover, and then provided tamoxifen. One control mouse and two Brg1/Brm double-mutant mice were followed for up to 17 days after the start of the 7-day tamoxifen-fortified chow feeding regimen. In the control mouse (Mouse 1), a normal heart rate, PR interval, QRS duration, and corrected QT intervals were observed at all of the time points collected (FIG. 2F). Both Brg1/Brm double-mutant mice (Mouse 2 and 3) demonstrated a normal heart rate, PR interval, QRS duration, and corrected QT interval at the baseline (prior to tamoxifen treatment) that were indistinguishable from the control (FIG. 2F). However, evidence of significant repolarization abnormalities were identified by ECG in the Brg1/Brm double-mutants by day 13 post-tamoxifen induction. For Brg1/Brm double-mutant Mouse 2, the QT interval was <50% of the R-R interval at baseline, whereas the QT interval was clearly >50% of the R-R interval at day 13, which is consistent with marked QT prolongation (FIG. 2F). Abnormal ST segment morphology, characterized by downsloping ST depression and T-wave inversion was also evident. This time point also was characterized by relative bradycardia and mild widening of the QRS complex. These abnormalities persisted until day 17 post-tamoxifen at which time the mouse developed apparent sinus arrest with a slow ventricular escape rhythm (FIG. 2F). This terminal rhythm progressed to complete cessation of electrical activity within hours. Brg1/Brm double-mutant Mouse 3 demonstrated abnormalities in both atrioventricular (AV) conduction and ventricular repolarization by ECG analysis at day 13. The AV conduction abnormalities initially manifested as prolongation of the PR interval (1^{st} degree AV block) (FIG. 2F) and progressed rapidly to complete heart block (3^{rd} degree AV block) with a slow junctional escape (FIG. 2F). This terminal rhythm progressed to complete cessation of electrical activity within minutes on day 13. This time point was characterized by repolarization abnormalities, initially manifest as markedly peaked (“hyperacute”) T waves, followed by ST segment depression and T wave inversion (FIG. 2F). The QRS interval also became mildly prolonged.

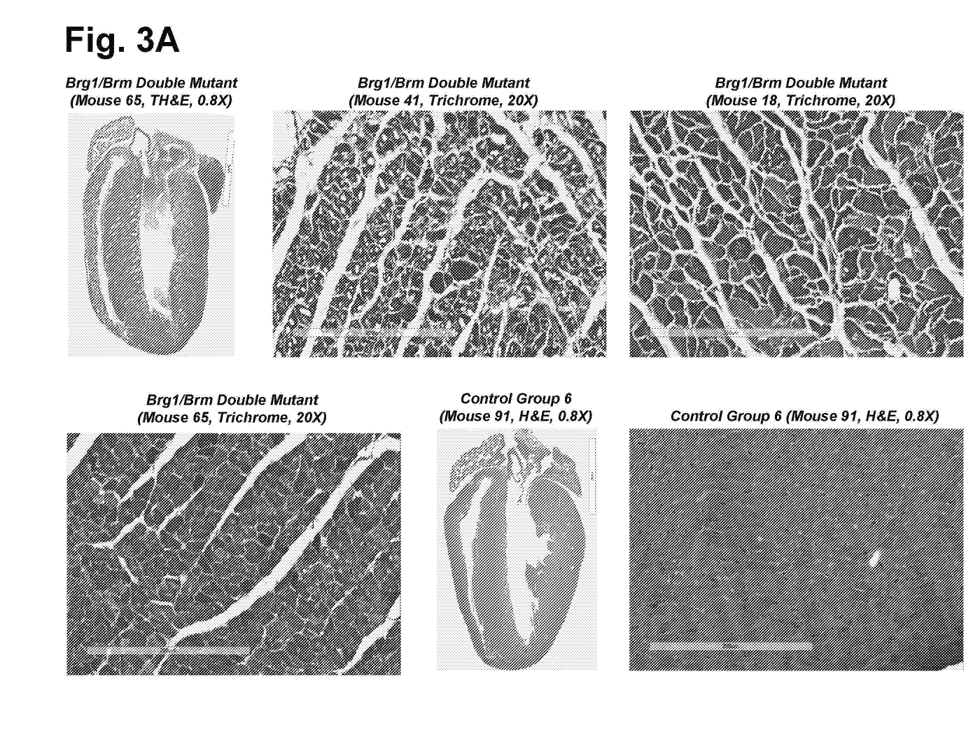

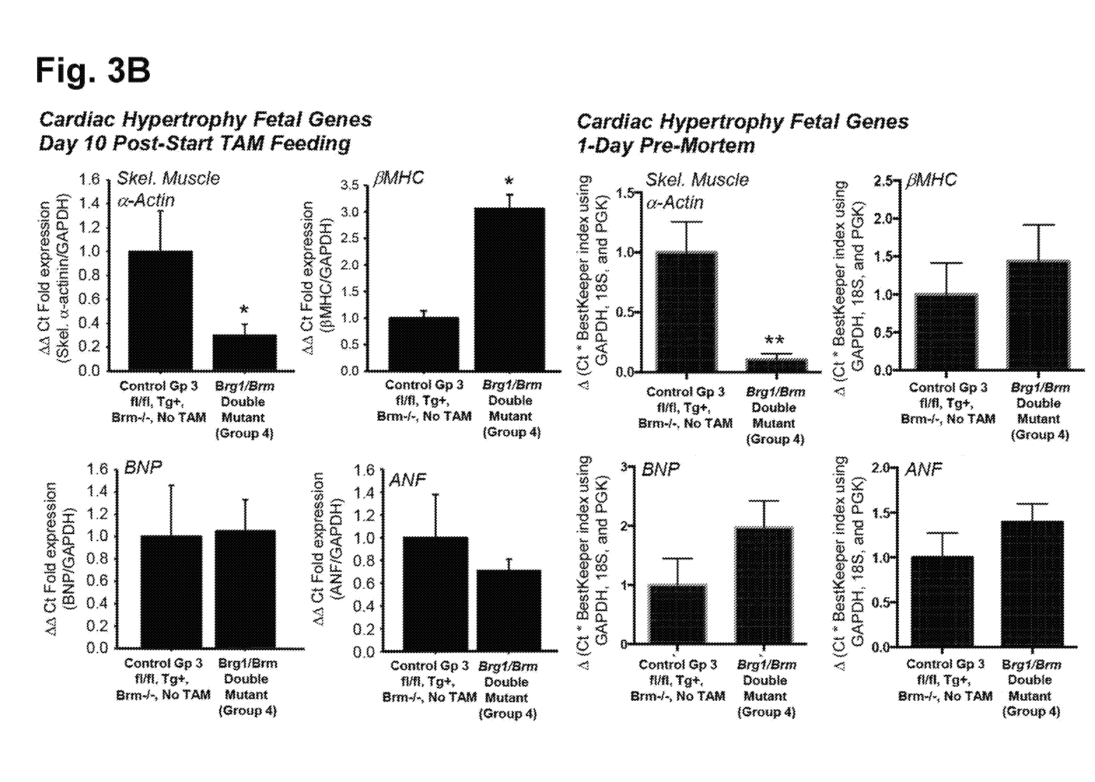

Brg1/Brm Double-Mutant Cardiomyocytes Undergo Mitophagy and Altered Mitochondrial Dynamics. Considering the severity of the cardiac phenotype and the rapid lethality of mice lacking Brg1 and Brm in their cardiomyocytes, the histopathology was surprisingly mild. Analysis of H&E-and Mason's Trichrome-stained sections revealed that the Brg1/Brm double-mutant phenotype ranged from relatively normal to moderate vacuolization without significant changes in fibrosis (FIG. 3A, FIGS. 14A-14D). Next, we assessed fetal cardiomyocyte gene expression because it is a typical pathological hypertrophic response that occurs during heart failure. Skeletal alpha-actin was significantly decreased at both time points in the Brg1/Brm double-mutant hearts compared to controls (FIG. 3B). β-MHC fetal gene expression was significantly elevated at day 10 post-tamoxifen induction (early time point) but not at 1-day pre-mortem (late time point) (FIG. 3B). In contrast, BNP and ANF were not changed significantly at either time point. These data are not representative of a typical pathologic hypertrophy/heart failure response.

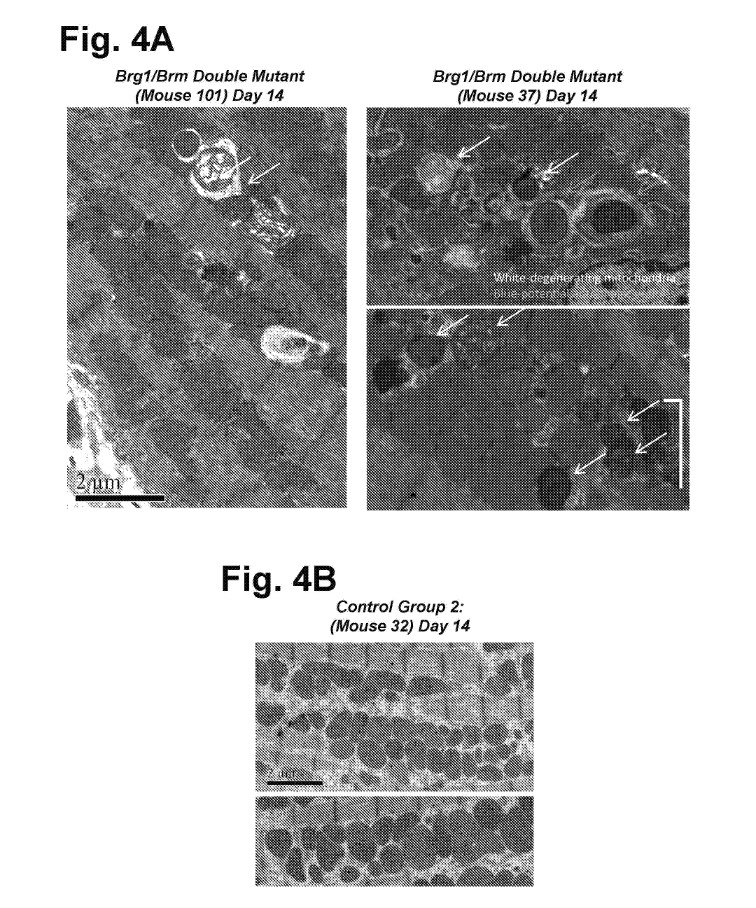

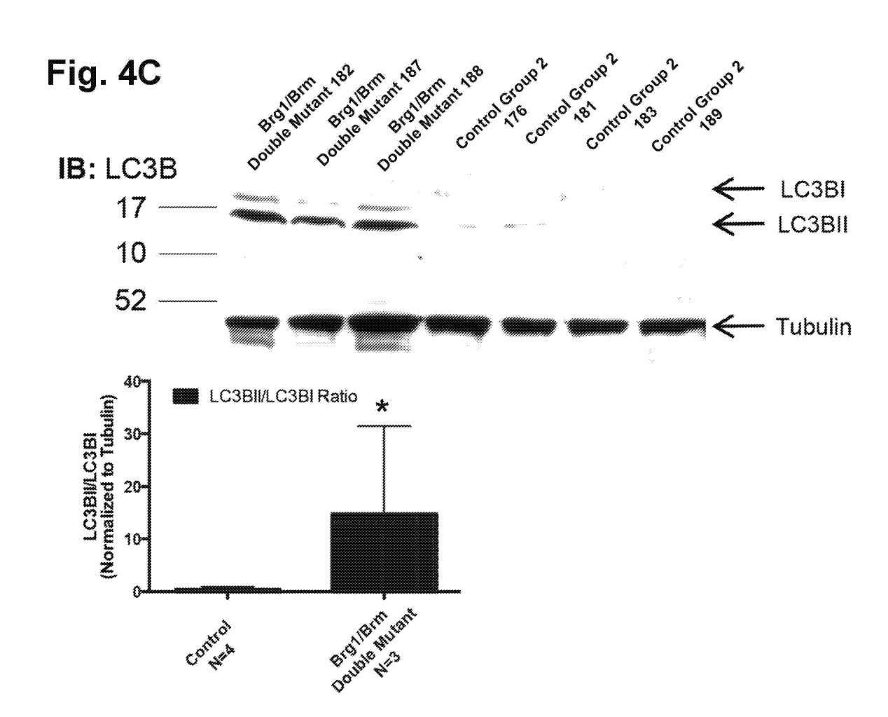

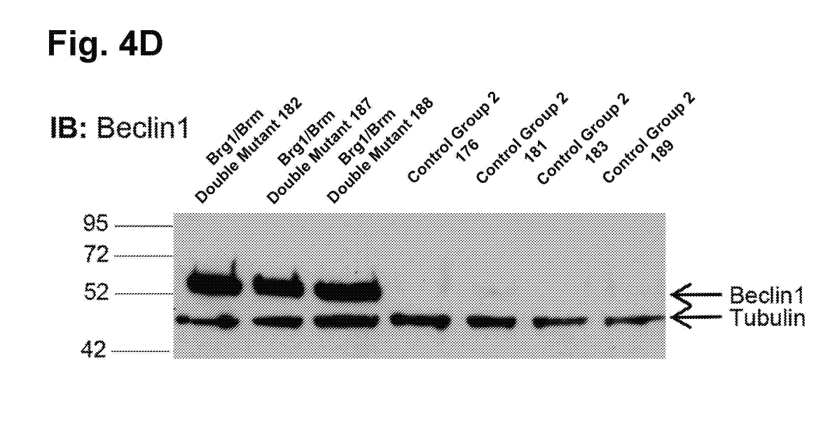

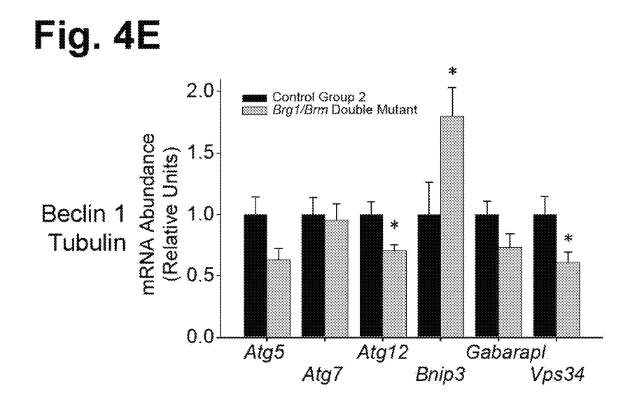

More detailed ultrastructural analysis of the heart by transmission electron microscopy (TEM) and analysis of autophagic flux revealed evidence of autophagy (FIG. 4). Both degenerating and double-membrane bound vesicles surrounding mitochondria in the interfibrillar areas were frequently observed in the Brg1/Brm double-mutant hearts but were undetectable in the control mice (FIGS. 4A and 4B). Autophagy is a conserved process whereby cytoplasmic components are degraded by the lysosome. This stepwise engulfment of cytoplasmic material by the phagophore, maturing into a double-membrane-bound vesicle, forms the autophagosome. Brg1/Brm double-mutant hearts exhibited a significant increase in autophagic flux, as illustrated by the LC3II:LC3I ratio by Western immunoblot after bafilomycin treatment, compared to control mice run in parallel (FIG. 4C). Increased autophagy was confirmed by Western immunoblot analysis of beclin (FIG. 4D).

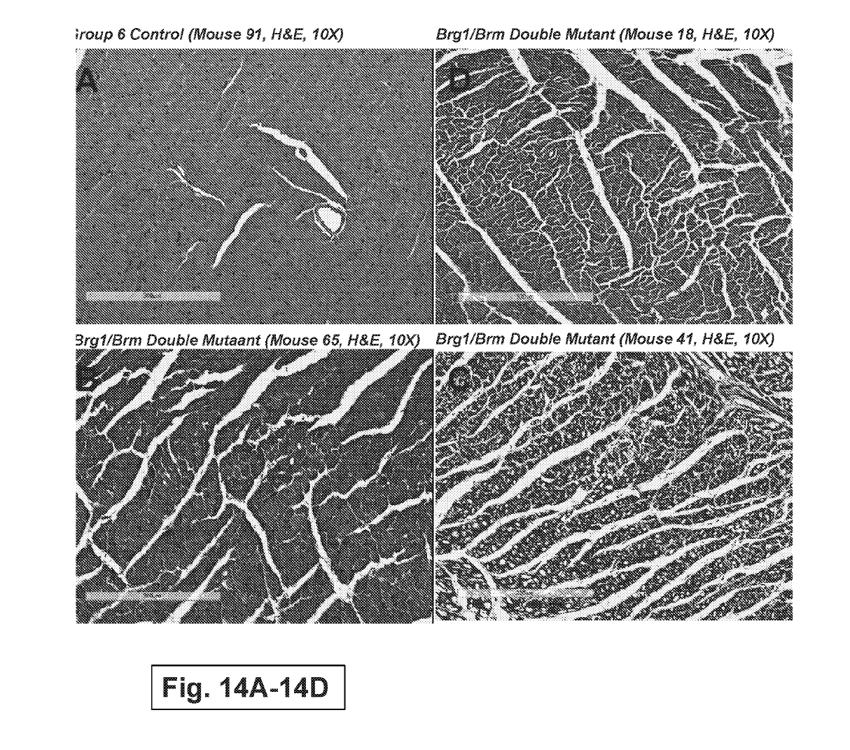

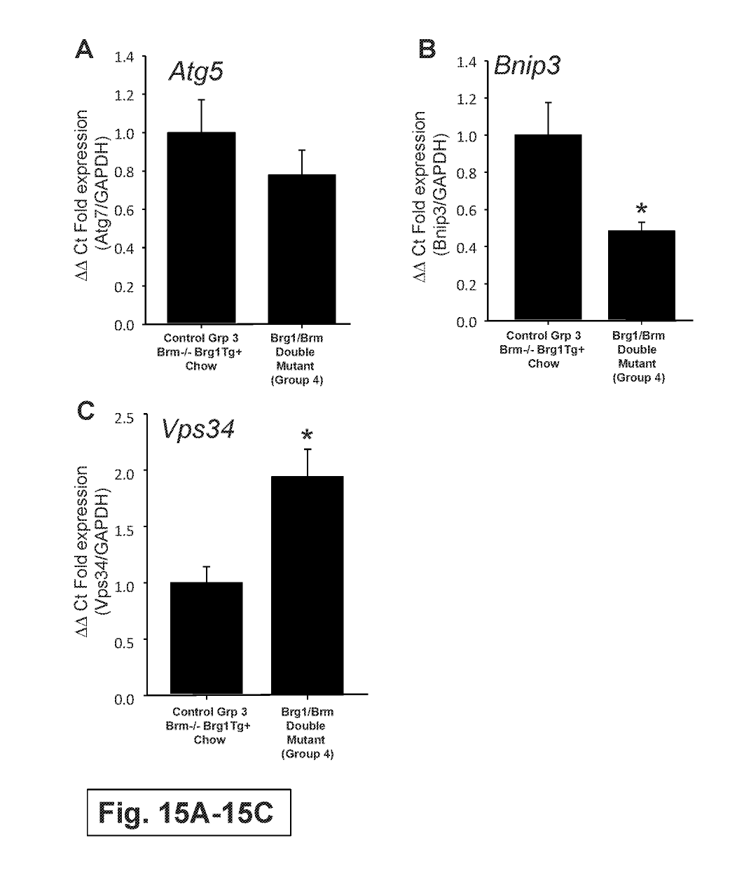

Transcriptional regulation of autophagy has been described, with a number of transcription factors that regulate Atg5, Atg7, Atg12, Bnip3, and Vps34 to enhance autophagy. RT-qPCR analysis of these genes demonstrated the Brg1/Brm double-mutant hearts had a significant increase in Bnip3 mRNA levels, while Atg12 and Vps34 were significantly decreased compared to control hearts at a late time point (FIG. 4E) but not an early time point (FIGS. 15A-15C). The transcriptional and epigenetic network regulating illustrates the diverse ways in which autophagy can be uniquely regulated, based on the stimulus. BNIP3, primarily located in the mitochondria as an integrated protein, perturbs outer membrane integrity, induces permeabilization of the inner mitochondrial membrane, and can stimulate mitophagy directly by triggering depolarization and Parkin recruitment. Taken together, these findings illustrate a novel role for BRG1 and BRM in the maintenance of cardiomyocyte autophagic flux, specifically related to the mitophagy as illustrated by TEM analysis and upregulation of Bnip3.

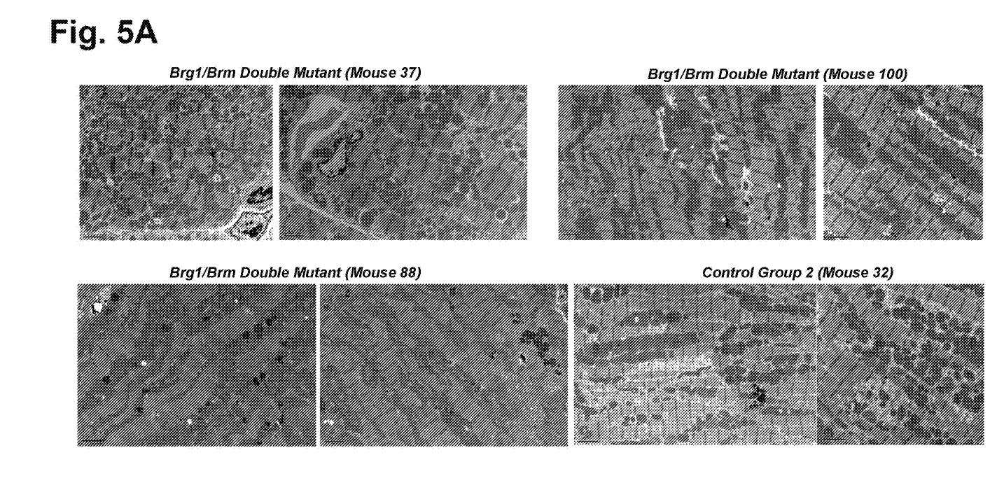

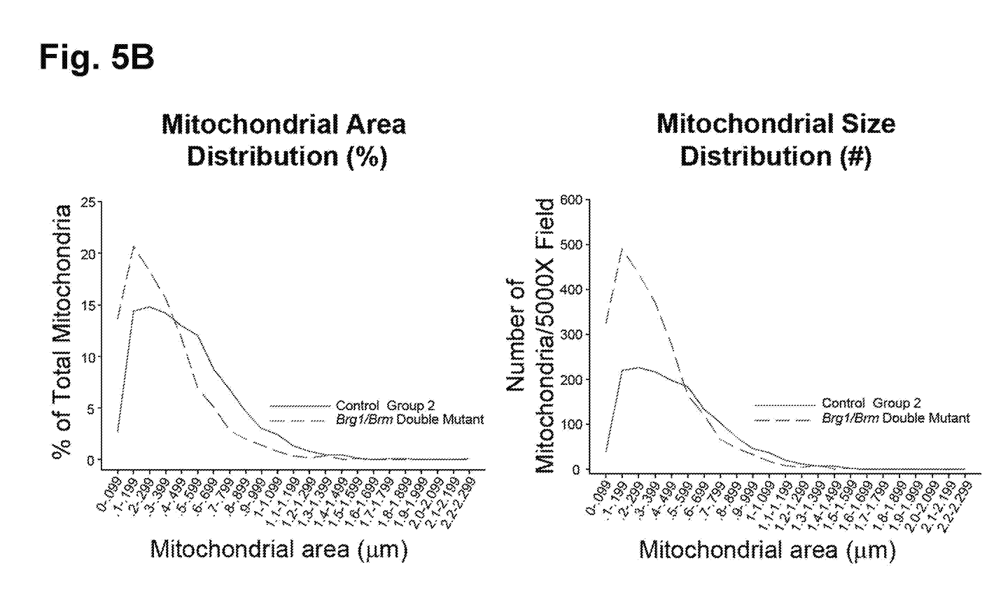

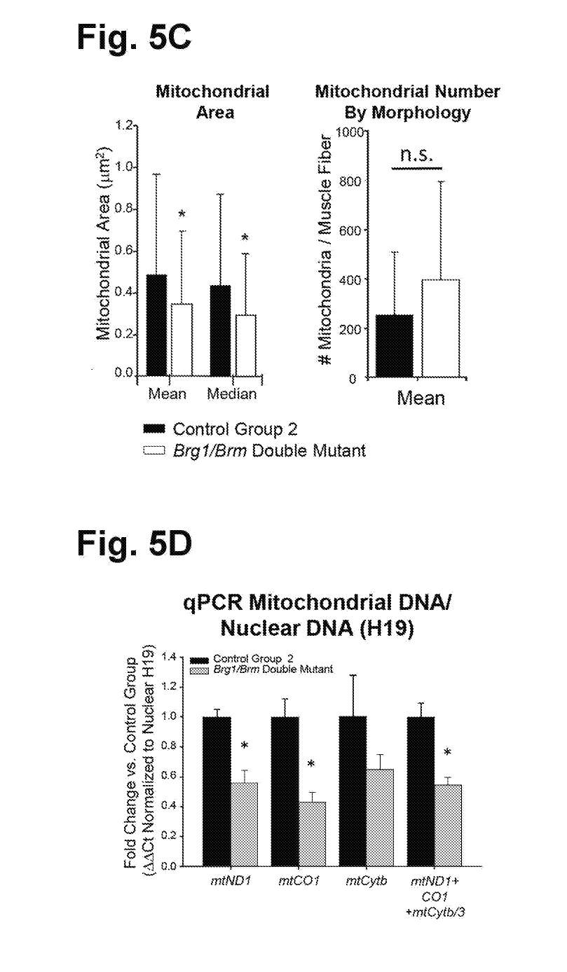

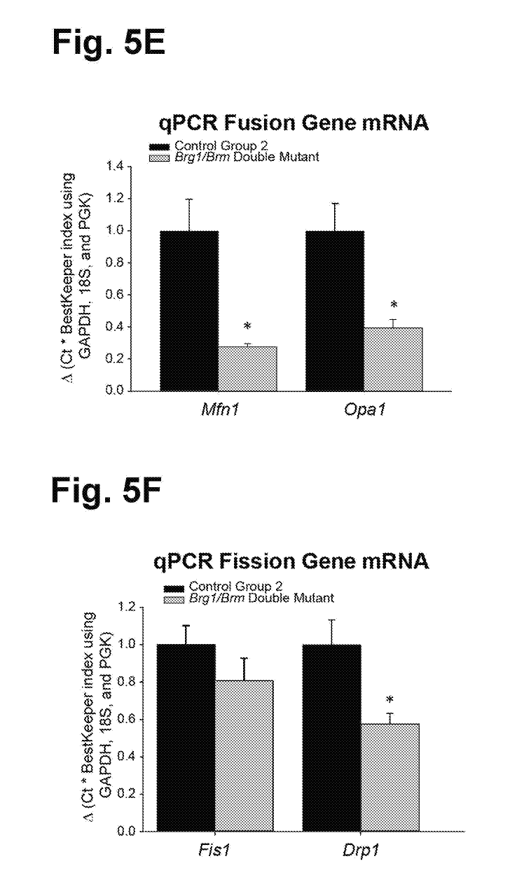

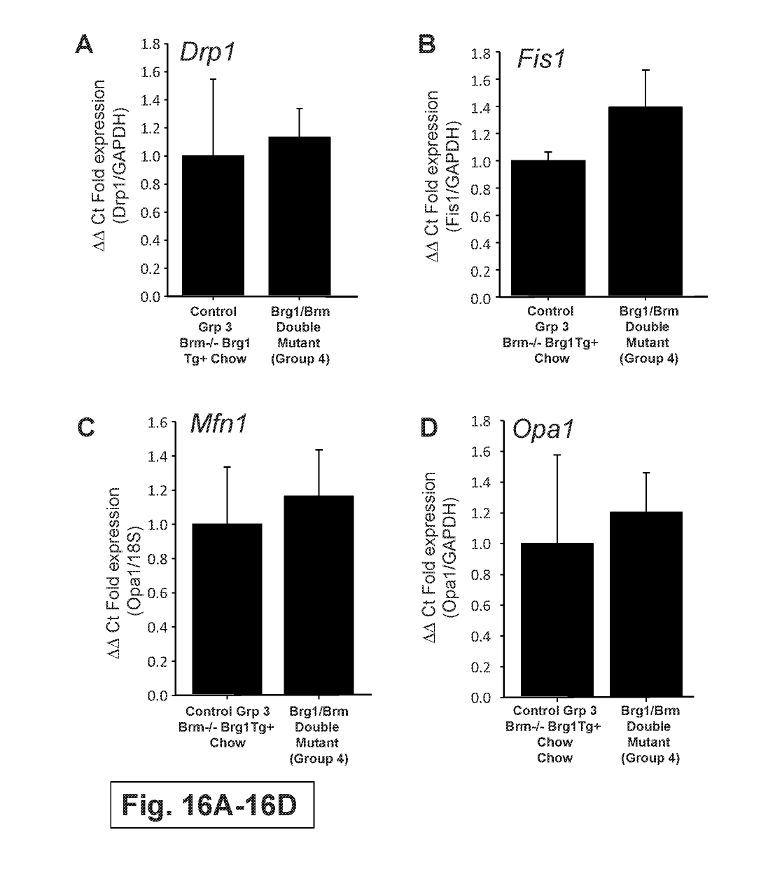

BNIP3 has been shown to induce mitochondrial fragmentation (fission) in cells. TEM revealed an apparent fragmentation of mitochondria in Brg1/Brm double-mutant hearts compared to controls (FIG. 5A). Analysis of mitochondria revealed that the number of small mitochondria identified in the Brg1/Brm double mutant hearts was skewed with a marked increase in small mitochondria with significantly smaller areas (FIGS. 5B and 5C). To put in context the apparent increase in small mitochondria, we determined mitochondrial number by qPCR of three mitochondrial encoded genes normalized to the nuclear encoded H19 and found a significant decrease in the number of mitochondria (FIG. 5D). The fusion and fission of mitochondria is a dynamic process that cells use for responding to mitophagy and maintaining protein quality. The regulation of mitochondrial dynamics by fusion and fission is regulated by four dynamin-related GTPases, including DRP1 and FIS1 (Fission) and mitofusins (e.g., MFN1) in the outer membrane and OPA1 in the inner membrane (Fusion). RT-qPCR analysis of these GTPases in the Brg1/Brm double-mutant hearts demonstrated significant decreases in Mfn1, Opa1 (FIGS. 5E, 16A-16D) and Drp1 (FIGS. 5F, 16A-16D). These findings demonstrate the interesting juxtaposition of increased mitophagy and mitochondrial fission, which has been shown to allow the selective elimination of damaged mitochondria by autophagy.

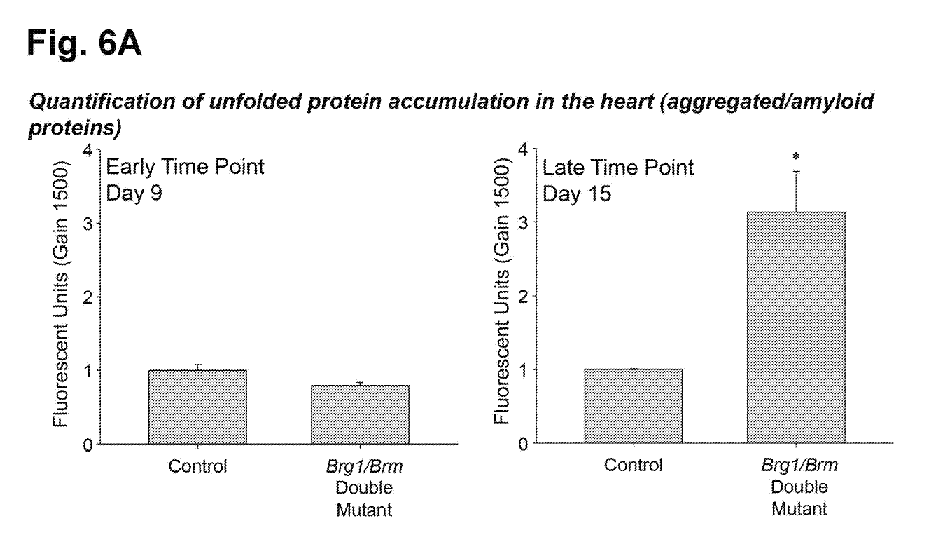

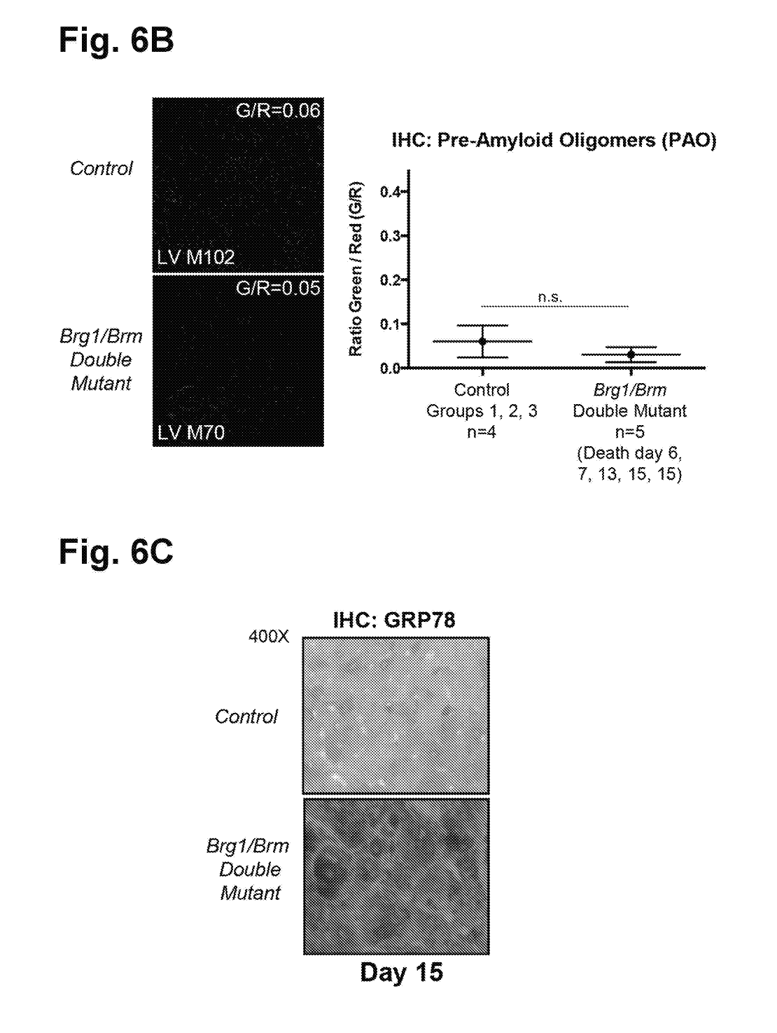

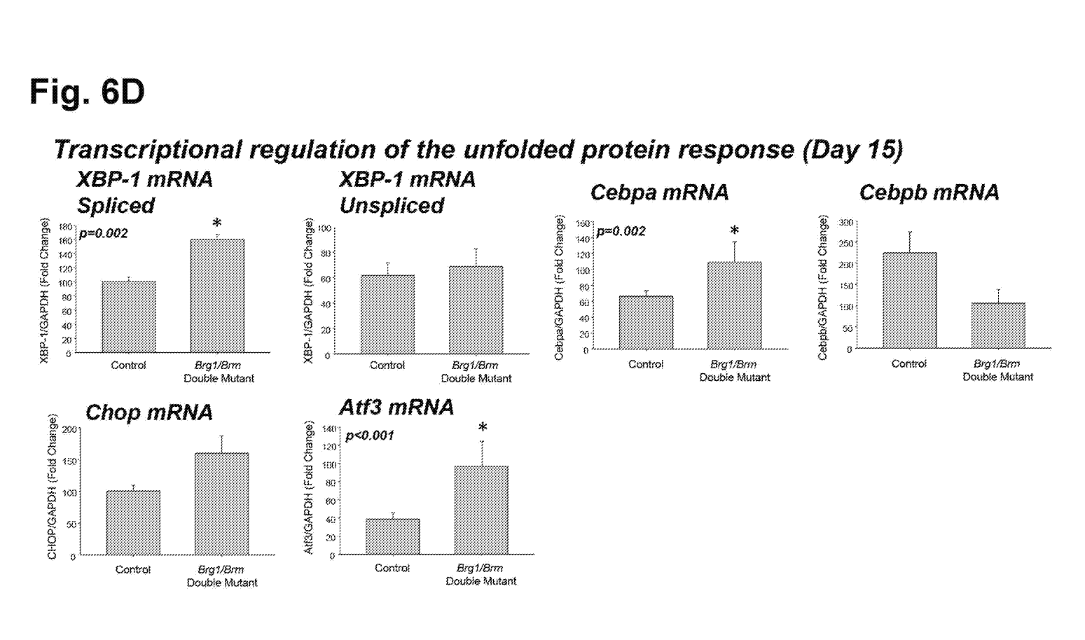

Activation of the UPR in Brg1/Brm Double-Mutant Cardiomyocytes. Histological analysis of the Brg1/Brm double-mutant hearts stained with H&E revealed stereotypical pink accumulation inside of the cardiomyocytes, resembling the unfolded proteins that accumulate in cardiac amyloidosis (FIGS. 14A-14D). Since the accumulation of unfolded proteins in mitochondria has been shown to induce mitophagy in neurons, we next investigated the presence of unfolded proteins using a colorimetric assay that has been used to identify amyloid and other unfolded proteins in experimental disease processes. At an early time point, Brg1/Brm double-mutant hearts did not have an increase in unfolded proteins (FIG. 6A). However, at later time points when mitophagy and altered fission and fusion were present (FIG. 4 and FIG. 5, respectively), a significant increase in unfolded proteins was present with a ˜3-fold increase compared to control hearts (FIG. 6A). However, an increase in the cytotoxic pre-amyloid oligomers (PAO) was not observed in cardiomyocytes. The detection of low levels of PAO present in cardiomyocytes (heavy chain myosin II) by immunoflourescence indicates that soluble PAOs were not present at early or late time points (days 6-15) in Brg1/Brm double-mutant hearts (FIG. 6B). These findings suggest that the protein aggregates detected in the heart at day 15 (FIG. 6A) accumulate very quickly without PAOs being present in significant amounts.

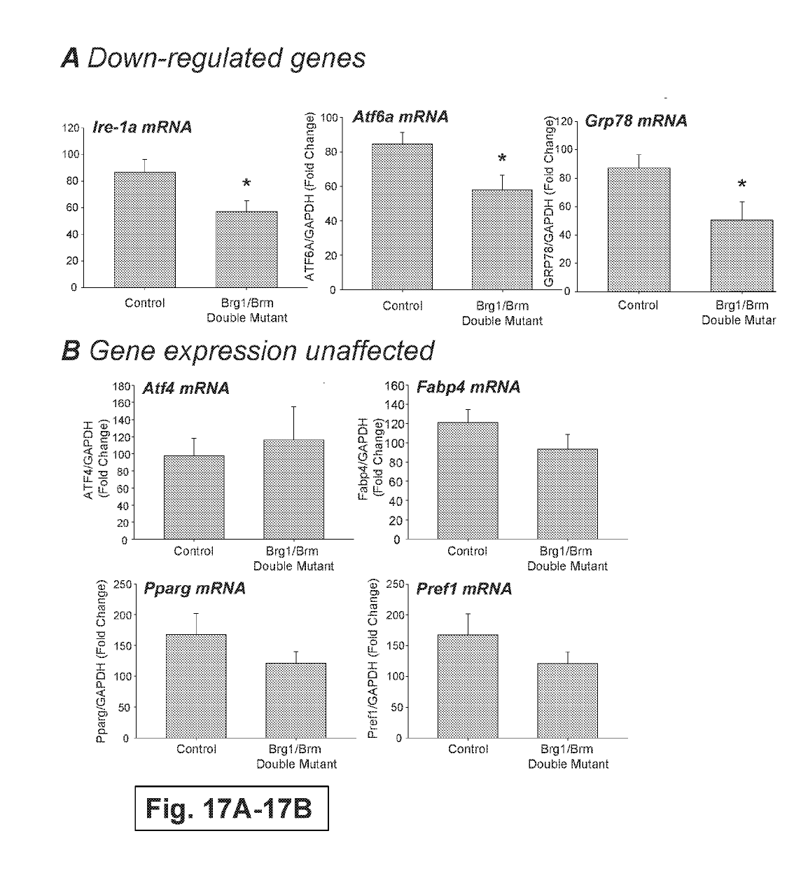

The heart's response to stress, including oxidized proteins, has been shown to induce the unfolded protein response (UPR), which includes the endoplasmic reticulum (ER) chaperone protein GRP78. Therefore, we next investigated the expression of GRP78 by IHC and identified that Brg1/Brm double-mutant mice had markedly increased GRP78 expression compared to controls (FIG. 6C) at the late time point where unfolded proteins were identified. The link between the UPR and cardiovascular disease is strengthening, but UPRs are diverse and include signaling by IRE-1, PERK, and ATF6. Identification of the spliced Xbp-1 by RT-qPCR is one way in which IRE-1 activity is measured. We identified that Brg1/Brm double-mutant hearts had a significant increase in the spliced Xbp-1 mRNA compared to control hearts (FIG. 6D). Downstream of the IRE1α-XBP-1 pathway, XBP1 binds the promoter of the C/EBPα (Cebpa) gene to increase its expression. Brg1/Brm double-mutant mice had significantly more Cebpa mRNA expression compared to controls (FIG. 6D). The significant increase in Brg1/Brm double-mutant heart Xbp-1 splice variant mRNA and Cebpa mRNA demonstrates the increased activity via IRE-1 signaling. The relationship between XBP1 and C/EBPβ (Cebpb) is a bit more complex, with evidence that C/EBPβ induces expression of) (BPI protein. Brg1/Brm double-mutant hearts had diminished Cebpb mRNA levels that were not significant but trended toward significance (FIG. 6D). The stress caused in the ER by the presence of unfolded proteins can also cause signaling through PERK, whereby PERK proteins dimerize and undergo autophosphorylation. Activation of ATF4 increases target gene expression, including the transcription factor CHOP (CCAAT/-enhancer-binding protein homologous protein), which itself induces the expression of Atf3 mRNA. Consistent with an increase in PERK activity, Brg1/Brm double-mutant hearts demonstrated an increase in Chop mRNA that trended toward significance and significantly higher Atf3 mRNA levels (FIG. 6D). While activation of ATF6 signaling was not directly measured, due largely to the deficiencies in reliable reagents, we determined the mRNA levels of other reported genes involved in the UPR. We found that Brg1/Brm double mutant hearts had either significant down-regulation of Ire-1a, Atf6a, and Grp78 reflecting compensatory responses to the clear GRP78 and IRE-1 signaling present, or no change in genes not as clearly related to the UPR in cardiomyocytes (FIG. 17).

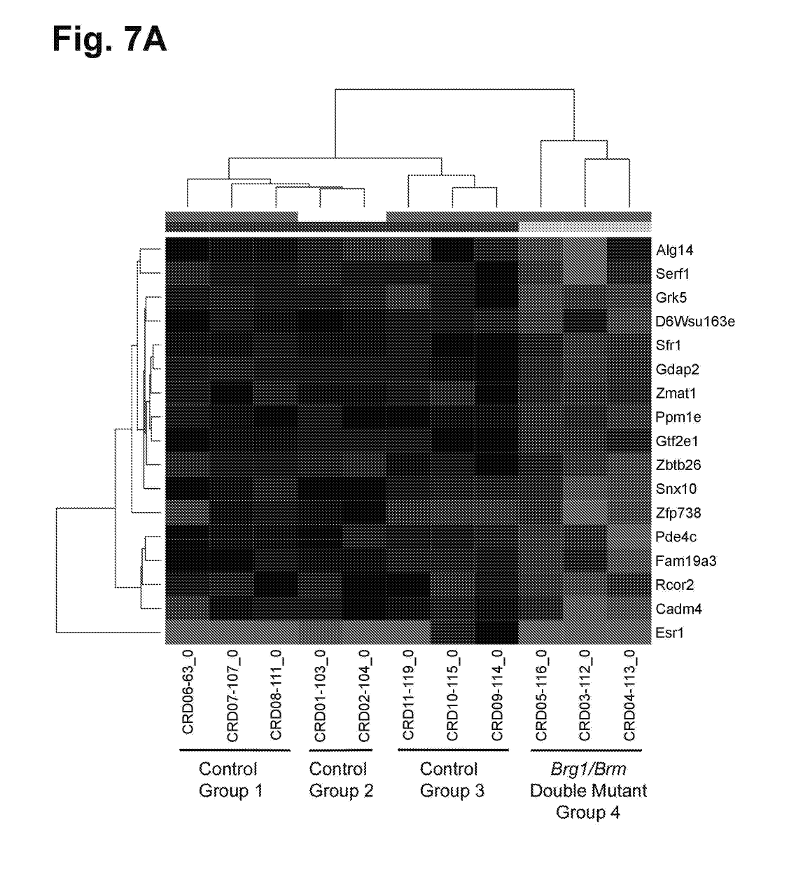

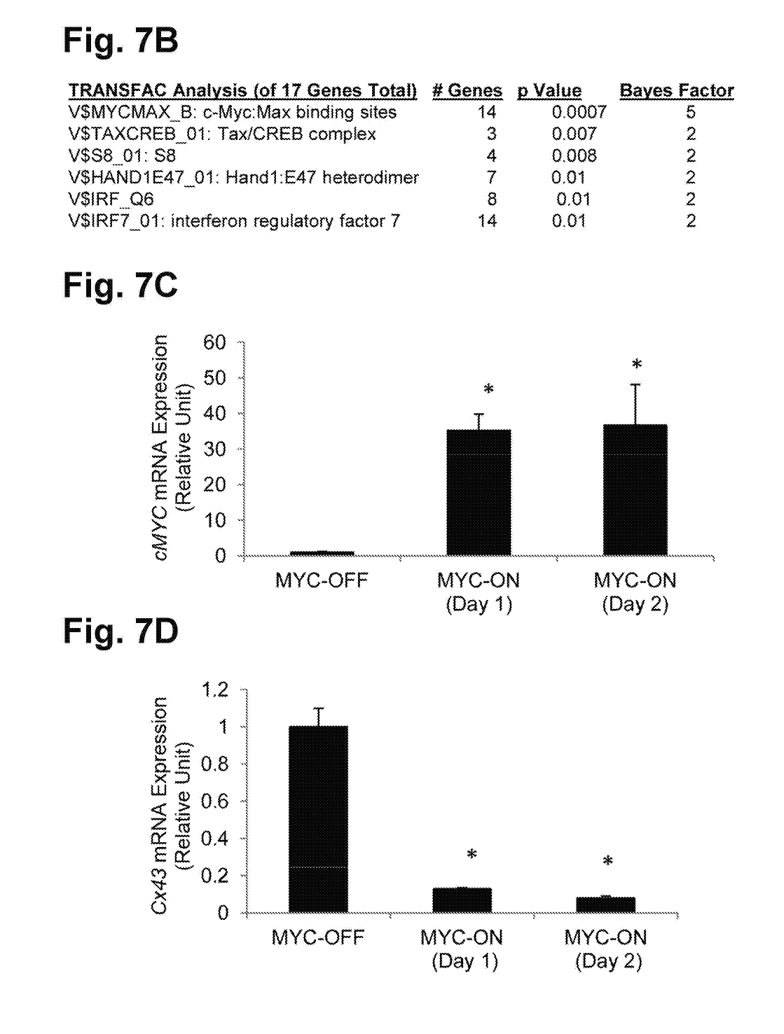

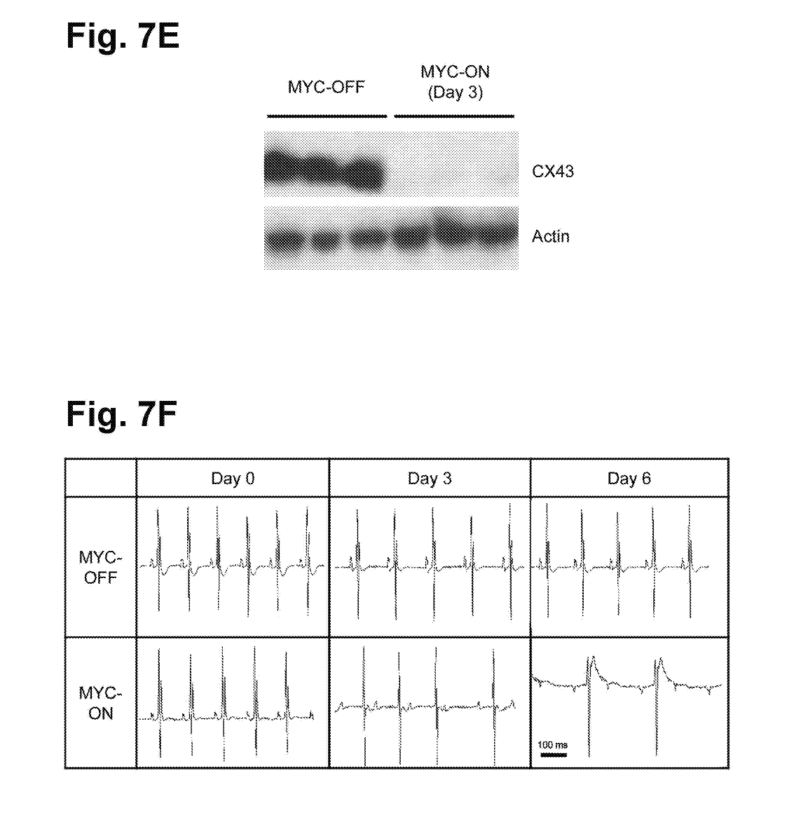

A Regulatory Mechanism Integrating SWI/SNF, c-Myc, and Cardiac Connexins. To further delineate the mechanisms underlying the pathogenesis of the heart disease caused by inducibly ablating Brg1 and Brm in cardiomyocytes, we performed RNA-seq transcriptome analysis on hearts from Brg1/Brm double-mutant mice at an early time point (day 9 post-tamoxifen induction) compared to multiple control groups. At this early time point, only 17 mRNAs were significantly different in the Brg1/Brm double-mutant hearts (FIG. 7A). An early time point was chosen to differentiate the direct role of BRG1/BRM in the disease process from an indirect, secondary response the cardiomyocytes might have had due to these primary events. The use of multiple control groups further increased the rigor of this study, which contributed to the relatively small number of significant genes. TRANSFAC analysis of these 17 genes identified c-MYC binding sites in 14 of the 15 named genes (p=0.0007, Bayes Factor 5) (FIG. 7B). This was particularly interesting given that SWI/SNF has been recently shown to play a role in acute leukemia maintenance and enhancer-mediated c-Myc regulation, where BRG1 was found to be required to maintain transcription factor occupancy for long-range chromatin looping interactions with the c-Myc promoter.

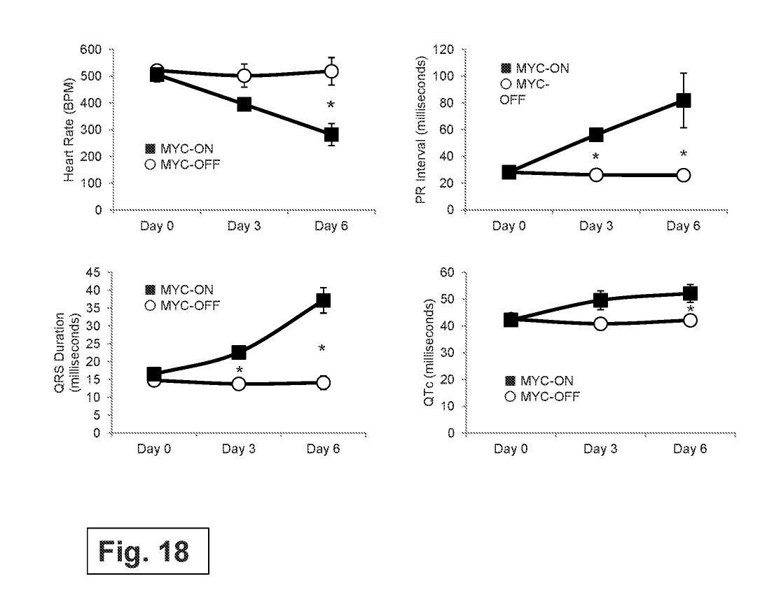

To evaluate the importance of c-Myc, which is associated with cardiac hypertrophy, we analyzed a previously described transgenic mouse line that inducibly overexpresses c-Myc in adult cardiomyocytes. RT-qPCR confirmed that DOX induces c-MYC expression within 24 hours of treatment (FIG. 7C). This c-MYC induction significantly decreased the expression of the cardiac connexin Cx43 at the mRNA (FIG. 7D) and protein (FIG. 7E) levels. ECGs of c-MYC-ON transgenic mice revealed cardiac dysfunction compatible with CX43 downregulation that was similar to what we observed in the Brg1/Brm double mutants (FIG. 7F and FIG. 18). These findings suggest that there is a functional link between SWI/SNF, c-Myc, and cardiac connexins that regulates conduction in cardiomyocytes.

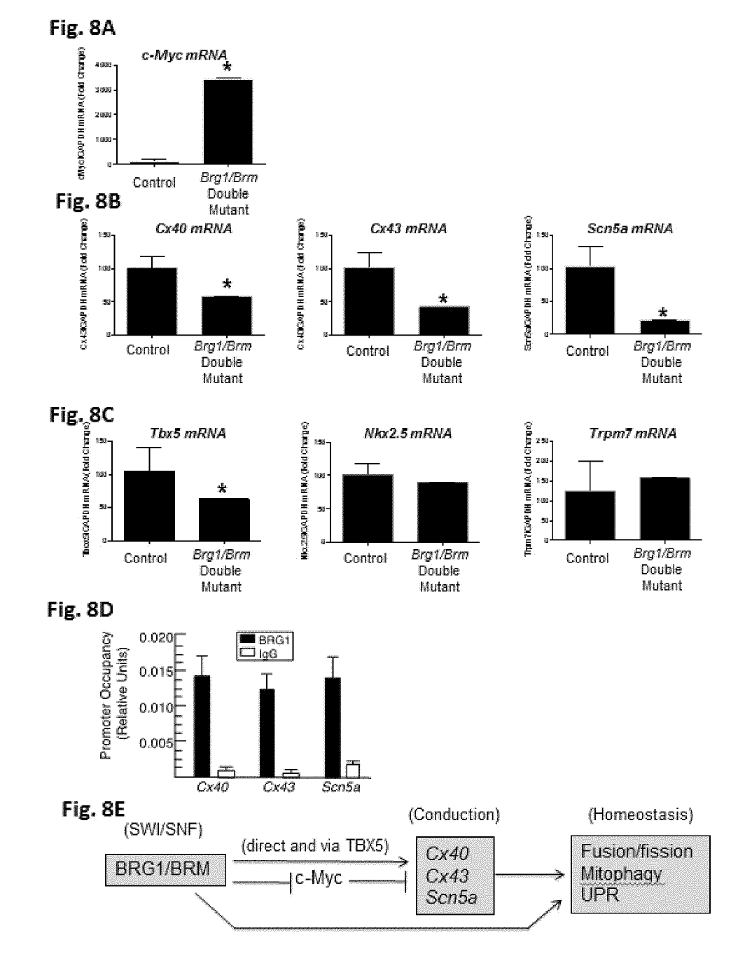

To test the hypothesis that SWI/SNF complexes participate in a cardiac conductance regulatory mechanism, we analyzed c-Myc, Cx40, Cx43, and Scn5a expression levels. Indeed, Brg1/Brm double-mutant hearts had significantly increased mRNA levels of c-Myc (FIG. 8A) and significantly decreased mRNA levels of Cx40, Cx43, and Scn5a (FIG. 8B). The cardiogenic transcription factors TBX5 and NKX2.5 can also be upregulated during HF and have been implicated in the upregulation of Cx40 and Cx43. BRG1 cooperates with these and other cardiogenic transcription factors during development, and Brg1/Brm double-mutant hearts had significantly lower levels of Tbx5 than controls (FIG. 8C).

During cardiac development, the cardiogenic transcription factors recruit SWI/SNF complexes to a number of target genes. To determine whether this is the case for electrical conduction in the adult heart, we performed quantitative ChIP assays and observed BRG1 occupancy at the promoter of the Cx40, Cx43, and Scn5a genes (FIG. 8D). This result indicates that BRG1 upregulates the conduction system directly (by binding to the target gene promoters) as well as indirectly (by inhibiting c-Myc expression and activating Tbx5 expression) (FIG. 8E).

The inducible cardiac Brg1/Brm double-mutant mouse model described here for the first time allowed the function of SWI/SNF complexes to be characterized in the adult heart. In a recent study, we demonstrated that Brg1/Brm double-mutant hearts had defects in the biosynthesis of unsaturated fatty acids/linoleic acid metabolism and in amino sugar and nucleotide sugar metabolism (glucose and myoinositol) at the early time point studied here (day 10 post-start TAM feeding) by non-targeted metabolomics analysis. Here we extend these findings to demonstrate pathological hypertrophic changes occurring early (day 10 post-start TAM feeding) (FIG. 2B), along with changes in gene expression associated with c-Myc (FIG. 7). How these early gene changes resulted in the changes in mitochondrial number and dynamics (fission and fusion), increase in autophagy and unfolded proteins, along with the unfolded protein response to result in a lethal arrhythmia in vivo illustrates the complexity of the overlapping roles that BRG1 and BRM have in the adult differentiated cardiomyocyte.

The Brg1/Brm double-mutant phenotype initially manifested as a prolongation of the PR interval (1^{st} degree AV block), which progressed to lethal arrhythmias resulting from AV conduction abnormalities. The communication between cardiomyocytes necessary for the heart's synchronous contraction occurs by gap junctions and ion channels. Conduction abnormalities between cardiomyocytes can lead to arrhythmias and sudden death. The role that potassium and sodium ion channels play in this process is evident when disease-causing mutations in these genes (KCNQ1, KCNH2 and SCN5A) lead to lethal arrhythmias due to alterations in the cells ability to repolarize (accounting for the prolonged QT interval by ECG). Maintenance of the gap junctions between cardiomyocytes is critical to maintaining conduction in the heart, with the distribution and function of the gap protein connexin 43 (CX43) and sodium channel Nav1.5 (encoded by SCN5A) critical to conduction. Inducible

Cx43 knockout in mice resulted in a heterogeneity of outcomes over a two-week period, with those experiencing arrhythmias having decreased Cx43 expression (and reduced Na current) with global heterogeneity, in contrast to those with arrhythmias. Several transcription factors have been implicated in regulating Nav1.5 and connexin 40, another important gap junction protein, including TBS5 and NKX2.5. SWI/SNF complexes, which utilize BRG1 and BRM as alternative catalytic subunits with ATPase activity, orchestrate the NKX2.5-directed contractile cardiomyocyte program in the sinoatrial node to enhance downstream TBS5 expression.

Although c-Myc is best known as a proto-oncogene in cancer, overexpression in quiescent cardiomyocytes is associated with cardiac hypertrophy. In addition to re-entering the cell cycle, c-Myc overexpressing cardiomyocytes downregulate CX43 expression, resulting in ventricular arrhythmias in as little as three days after c-Myc expression is increased (FIG. 7F, FIG. 18). The Brg1/Brm double mutant mice had a signature indicating that the strongest relationship between the differentially expressed genes was that they had c-MYC binding sites in their promoters, which suggests a possible mechanism in the present study. Our findings that BRG1 and BRM inhibit c-Myc expression and antagonize c-Myc in terms of Cx43 expression is reminiscent of their opposing functions in cancer where BRG1 and BRM function as tumor suppressors and c-MYC functions as a proto-oncogene. In fact, BRG1 has been reported to antagonize c-Myc activity in primary tumor cells. c-MYC overexpression in the heart is also associated with alterations in cardiac metabolism and mitochondrial biogenesis.

Cardiac c-MYC regulates cardiac glutaminolysis, a process that is associated with microvascular rarefaction/ischemia. Glutaminolysis is a series of reactions that are commonly utilized in tumor cells that enable them to utilize amino acids as an additional source of energy, used when glycolytic energy production is low. Since glutaminolysis can occur in the presence of high ROS, it is a valuable way to survive oxygen deprivation and in the heart has been shown to reprogram mitochondrial metabolism via c-MYC, which we recently showed is altered in the Brg1/Brm double mutant hearts. c-MYC also regulates mitochondrial dynamics with evidence that c-Myc deficiency results in Drp1 deregulation and hyper-fission in fibroblasts, paralleling the phenotype seen here in Brg1/Brm double mutant hearts. Other components of the SWI/SMF core subunit (BAF155 in a CARM-dependent manner) have been found to direct unique chromatin regions (c-MYC pathway genes). Since c-MYC is one of 5 hub nodes in transcriptional networks having a close relationship with heart failure, it is possible that this is an underlying cause of the defects seen downstream of Brg1/Brm deletion in the current study.

The lethal arrhythmias seen in the Brg1/Brm double mutant mice may have other more direct causes, including the unfolded protein response itself. In human heart failure, increases in SCN5A alternative splice variants become trapped in the endoplasmic reticulum. Recent studies have identified that both activators of abnormal SCN5A mRNA splicing (e.g., Ang II or hypoxia) or increasing expression of these SCN5A variants directly, resulted in activation of UPR effectors, including PERK, calreticulin, and CHOP. Induction of the SCN5A variants resulted in concomitant decreases in Na+ current in vitro resulting in PERK destabilization of SCN5A and Kv4.3 channel (mRNA) but not the transient receptor potential cation channel M7 (TRPM7). Since unfolded proteins were identified at later time points in Brg1/Brm double-mutant mice, it is temporally possible that they could have contributed to the lethal arrhythmias identified recently by altering the Na+ channel currents by inducing alternative splicing of Scn5a.

Mitochondrial fusion and fission are processes essential for the preservation of normal mitochondrial function. In heart failure, OPA1 is important for maintaining normal cristae structure and function, while preserving the inner membrane structure and for protection against apoptosis. Confocal and TEM analysis has demonstrated that failing hearts have small, fragmented, mitochondria, consistent with decreased fusion. Reducing OPA1 or MFN1 in cardiomyocytes results in increased mitochondrial fragmentation. The transcriptional regulation of genes involved with mitochondrial fusion in cardiomyocytes (i.e., OPA1 and MFN1) as well as fission (e.g., DRP1) have been recently shown to be under the control of PGC-1α/β by coactivated the estrogen-related receptor a While the SWI/SNF complex component BAF60a has been shown to support PGC-1α co-activation, regulation of mitochondrial fission by Brg1/Brm demonstrated here has previously not been described. Smaller mitochondria and increased total number of mitochondria were also observed in our cMYC-ON mice, suggesting a role for MYC in this context.

The regulation of autophagy, a conserved process utilizing the lysosome to degrade cytosolic components, has largely been described solely as a cytosolic event. Our understanding of the transcriptional and epigenetic control of autophagy is evolving, whereby multiple transcription factors and epigenetic regulators have been shown to be involved. Transcription factors including C/EBPβ, E2F1, FOXO1/3, HIF1, and STAT3 regulate the transcription of BNIP3, for example, which independently in the heart, BNIP3 expression is increased after myocardial infarction and in response to chronic pressure overload hypertrophy. Increasing cardiomyocyte BNIP3 itself induces mitochondrial autophagy (mitophagy), which has an essential function in mitochondrial pruning. Enhanced mitophagy was seen Brg1/Brm double mutant mice, with a unique increase in Bnip3. The chromatin-remodeling complex SWI/SNF is essential for HIF1 activity, with HIF-1-dependent mitophagy reported as a metabolic adaptation to stress.

### Example 2

### Non-Targeted Metabolomics of Brg1/Brm Double-Mutant Cardiomyocytes Reveals a Novel Role for SWI/SNF Complexes in Metabolic Homeostasis

Mammalian SWI/SNF chromatin-remodeling complexes utilize either BRG1 or BBRM as alternative catalytic subunits to alter the position of nucleosomes and regulate gene expression. Genetic studies have demonstrated that WI/SNF complexes are required during cardiac development and also protect against cardiovascular disease and cancer. However, Brm constitutive null mutants do not exhibit a cardiomyocyte phenotype and inducible Brg1 conditional mutations in cardiomyocytes do not demonstrate differences until stressed with transverse aortic constriction, where they exhibit a reduction in cardiac hypertrophy. We recently demonstrated the overlapping functions of Brm and Brg1 in vascular endothelial cells and sought here to test if this overlapping function occurred in cardiomyocytes. Brg1/Brm double mutants died within 21 days of severe cardiac dysfunction associated with glycogen accumulation and mitochondrial defects based on histological and ultrastructural analyses. To determine the underlying defects, we performed nontargeted metabolomics analysis of cardiac tissue by GC/MS from a line of Brg1/Brm double-mutant mice, which lack both Brg1 and Brm in cardiomyocytes in an inducible manner, and two groups of controls. Metabolites contributing most significantly to the differences between Brg1/Brm double-mutant and control-group hearts were then determined using the variable importance in projection analysis. Increased cardiac linoleic acid and oleic acid suggest alterations in fatty acid utilization or intake are perturbed in Brg1/Brm double mutants. Conversely, decreased glucose-6-phosphate, fructose-6-phosphate, and myoinositol suggest that glycolysis and glycogen formation are impaired. These novel metabolomics findings provide insight into SWI/SNF-regulated metabolic pathways and will guide mechanistic studies evaluating the role of SWI/SNF complexes in homeostasis and cardiovascular disease prevention.

Mammalian SWI/SNF (switching defective/sucrose nonfermenting) complexes are comprised of 9-12 subunits including either BRG1 (brahma-related gene 1, also known as SMARCA4) or BRM (brahma, also known as SMARCA2) as alternative catalytic subunits with DNA-dependent ATPase activity. These chromatin-remodeling complexes are recruited by pioneer transcription factors to the promoters of numerous target genes, where they slide or evict nucleosomes in an ATP-dependent manner to either activate or suppress RNA Polymerase II occupancy and transcription. In addition to BRG1 or BRM, SWI/SNF complexes contain 8-11 additional subunits called BAFs (BRG1/BRM associated factors with a number referring to the protein molecular mass) that contribute to chromatin remodeling. Due to the combinatorial assembly of different BAFs, there are potentially many distinct SWI/SNF complexes that vary in subunit composition and function.

SWI/SNF complexes play an important role in cardiovascular development. In humans, mutations in several genes encoding SWI/SNF subunits including BRG1 and BRM result in congenital syndromes that exhibit highly penetrant cardiac defects. In mouse models, Baf250a, Baf180, and Baf60c constitutive null mutants exhibit a variety of cardiac defects including abnormal looping, hypoplastic ventricles, shortened outflow tracts, and septal defects that result in embryonic lethality. Although Brg1 constitutive null mutants die prior to organogenesis, conditional null mutations of Brg1 in the developing endocardium, myocardium, or myocardial progenitor cells of the secondary heart field result in a variety of defects including hypoplastic ventricles, thin myocardium, shortened outflow tract, lack of septum, and hypotrebeculation that also culminate in embryonic lethality. An essential role for BRG1 in cardiomyocyte development is consistent with it physically interacting with the cardiogenic transcription factors TBS5, GATA4, and NKX2.5, and the ability of BAF60c, TBS5, and GATA4 to differentiate non-cardiac mesoderm into beating cardiomyocytes. While Brg1 is essential for cardiomyocyte development, it is dispensable for cardiomyocyte viability in the adult animal. However, an inducible conditional mutation of Brg1 in adult cardiomyocytes did result in decreased hypertrophy following transverse aortic constriction to pressure overload the heart. In contrast, Brm constitutive null mutants are viable and do not exhibit a discernible cardiomyocyte phenotype compared to sibling wild-type controls. The combined role of Brg1 and Brm in the adult cardiomyocyte has not previously been described.

The redundancy of Brg1 and Brm in vascular endothelial cells (VECs) within the adult heart has recently been reported. Brg1 is required by VECs during embryonic development, whereas Brm is dispensable. Furthermore, the Brg1 mutant phenotype of developing VECs is not exacerbated by Brm deficiency. However, an inducible conditional mutation of Brg1 in VECs from adult mice did not result in an observable phenotype. The lack of an adult phenotype was found to be due to the redundancy of Brg1 and Brm in the adult VECs within the heart, as double mutants died within 30 days of inducing the Brg1 deletion. Absence of Brg1 and Brm resulted in VEC apoptosis, vascular leakage, intra-cardiac dissection, and secondary cardiomyocyte cell death due to ischemia.

Considering that Brg1 and Brm are functionally redundant in adult VECs, we hypothesized that they may also have redundant functions in adult cardiomyocytes. To test this hypothesis, we analyzed Brg1/Brm double mutants where Brg1 could be mutated exclusively in cardiomyocytes in an inducible manner. Indeed, double mutants were not viable and their hearts exhibited signs of metabolic dysfunction. Therefore, we sought to characterize metabolomic changes in double mutants compared to controls. Recent advances in technology have afforded a more comprehensive analysis of a tissue's metabolome. Both targeted and non-targeted mass spectrometry based approaches have become common, with non-targeted methods being particularly valuable to explore phenotypes that involve many metabolites in a relatively unbiased manner. Non-targeted technologies have been used to characterize genetic diseases that result in altered metabolism of carbohydrates, lipids, and amino acids. The present studies demonstrate that BRG1 and BRM are required for metabolic homeostasis. Brg1/Brm double-mutant hearts exhibited altered metabolism of fatty acids (e.g., increased cardiac oleic and linoleic acids), glucose (e.g., decreased glucose-6-P, fructose-6-P, myoinositol), and amino acids. Both fatty acid and glucose substrate utilization has been shown to be a critical regulator of the heart's resiliency in the face of cardiac disease, with resulting fatty acid and glucose toxicity identified when there is an imbalance matched to the stressor. These studies demonstrate the critical role that BRG1 and BRM play in regulating fatty acid and glucose metabolism in the intact adult cardiomyocyte, likely by supporting the activity of multiple nuclear receptors implicated in regulating fatty acid and glucose utilization in the heart (e.g., PPAR, PGC1a).

Mouse Lines. The αMHC-Cre-ERT mice [also known as B6.Cg-Tg(Myh6-cre/Esr1)1JmkJ or αMHC-MerCreMer] were obtained from The Jackson Laboratory (#005657, Bar Harbor, Me.) and genotyped. The Brg1 conditional mutant mouse line and Brm constitutive mutant mouse line have been described previously. Genotyping of the Brg1 foxed and Dfloxed alleles and the Brm mutation was carried out by PCR.

Tamoxifen Induction of Brg1 Mutation. To induce the Brg1 conditional mutation in adult cardiomyocytes, 3-6 month old male and female mice were provided rodent chow containing tamoxifen (Sigma-Aldrich #T5648, St. Louis, Mo.) over a 7-day period. The route of delivery and dose were selected to minimize a previously described artifact caused by high doses of tamoxifen in the presence of the αMHC-Cre-ERT transgene. Briefly, 500 mg of tamoxifen was mixed with 1 kg of ground-up rodent chow and then mixed with water, kneaded into pellets, and dried in a hood. Provided to mice ad libitum, the dose was estimated to be 80 mg/kg/day. After the 7-day treatment period, the tamoxifen-fortified chow was removed and replaced with the same chow lacking tamoxifen.

Echocardiography. Conscious cardiac transthoracic echocardiography was performed on mice at the indicated time points using a VisualSonics Vevo 2100 ultrasound biomicroscopy system (VisualSonics, Inc., Toronto, Ontario, Canada) as previously described. Two-dimensional M-mode echocardiography was performed in the parasternal long-axis view at the level of the papillary muscle on loosely restrained mice. Anterior and posterior wall thickness was measured as distance from epicardial to endocardial leading edges. Left ventricular internal diameters were also measured. Left ventricular systolic function was assessed by ejection fraction (LV EF %=[(LV Vol; d-LV Vol; s/LV Vol; d) 9 100] and fractional shortening (% FS=[(LVEDD−LVESD)/LVEDD]9 100). Investigators were blinded to mouse genotype from collection through waveform measurements. Each measurement represents the average of three cardiac cycles from each mouse.

Histopathological and Ultrastructural Analyses. Histology was performed by fixing heart tissues in 4% paraformaldehyde, embedding in paraffin, cutting 5-μm sections, and staining sections with hematoxylin and eosin (H&E) according to standard procedures. For transmission electron microscopy (TEM), heart tissues were fixed in 2% paraformaldehyde and 2.5% glutaraldehyde in 0.15 M sodium phosphate buffer (pH 7.4) overnight and then postfixed with 1% osmium tetroxide in 0.15 M sodium phosphate buffer. Samples were dehydrated with increasing concentrations of ethanol, infiltrated and embedded in Polybed 812 epoxy resin (Polysciences, Warrington, Pa.), and 70-nm ultrathin sections were cut with a diamond knife. Sections were mounted on 200-mesh copper grids and stained with 4% aqueous uranyl acetate and Reynold's lead citrate. Sections were observed with a LEO EM910 transmission electron microscope operating at 80 kV (LEO Electron Microscopy, Thornwood, N.Y.) and photographed with a Gatan-Orius SC1000 CCD Digital Camera and Digital Micrograph 3.11.0 (Gatan, Pleasanton, Calif.).

RT-qPCR Analysis. RNA from double-mutant and control hearts was prepared using Trizol reagent (Life Technologies) and reverse transcribed using random hexamers and SuperScript II RT (Life Technologies) according to standard procedures. Pfkfb1 was amplified using the following primers (5′GAGTG CAAGACCACGTTCAA3′ (SEQ ID NO:140) and 5′GGAGCTGATGCTTT GAGACC3′ (SEQ ID NO:141) at 300 nM final concentration for each) and Power SYBR Green Master Mix (Life Technologies) under the following cycling parameters (95° C. 45 s, 60° C. 30 s, 72° C. 45 s). Dissociation curves and agarose gels demonstrated a single PCR product in each case without primer dimmers. Gapdh was amplified using a TaqMan assay (Life Technologies) and default cycling parameters. Negative control reactions lacking RT yielded little or no Pfk or Gapdh signal, and relative expression levels were determined using the DDCt method.

Metabolomics Determination by GC-MS Instrumentation. Cardiac tissue was flash frozen in a liquid nitrogen cooled biopress, a fraction of it weighed (*25-50 mg wet weight), then the finely cut up tissue quickly added to fresh pre-made buffer (50% acetyl-nitrile, 50% water, 0.3% formic acid) at a standard concentration of 25 mg/475 mcl buffer then fully homogenized on ice for 10-25 s and placed on dry ice/stored at −80° C. The samples were “crash” deprotonized by methanol precipitation and spiked with D27-deuterated myristic acid (D27-C14:0) as an internal standard for retention-time locking and dried. The trimethylsilyl (TMS)-D27-C14:0 standard retention time (RT) was set at *16.727 min. Reactive carbonyls were stabilized at 50° C. with methoxyamine hydrochloride in dry pyridine. Metabolites were made volatile with TMS groups using N-methyl-N-(trimethylsilyl) trifluoroacetamide or MSTFA with catalytic trimethylchlorosilane at 50° C. GC/MS methods were carried out with a 6890 N GC connected to a 5975 Inert single quadrupole MS (Agilent Technologies, Santa Clara, Calif.). The two wall-coated, open-tubular (WCOT) GC columns connected in series are both from J&W/Agilent (part 122-5512), DB5-MS, 15 meters in length, 0.25 nun in diameter, with a 0.25-μm luminal film. Positive ions generated with conventional electron-ionization (EI) at 70 eV are scanned broadly from 600 to 50 m/z in the detector throughout the 45 min cycle time.

Data were acquired using an MSD ChemStation (Agilent Technologies) by identifying metabolites on their mass fragmentation patterns and RT. Raw data formatted files were exported for further analysis in Automatic Mass Spectral Deconvolution and Identification Software or AMDIS (freeware developed at National Institute of Standards and Technology or NIST). Deconvoluted spectra are annotated as metabolites, to the extent possible, using an orthogonal approach that incorporates both RT from GC and the fragmentation pattern observed in EI-MS. Peak annotation is based primarily on our own RT-locked spectral library of metabolites. The library is built upon the Fiehn GC/MS Metabolomics RTL Library (Agilent, part number G1676-90000), Golm Metabolome Library (Max Planck Institute of Molecular Plant Physiology, Golm, Germany), the Wiley 9th-NIST 2011 commercial library (Agilent G1730-64000), and other spectral libraries. Once annotation was complete, a cross-tabulated spreadsheet was created, listing the integrated peak area for each metabolite versus sample identity. This was accomplished using a custom Visual Basic program in Microsoft Excel that grouped peaks across samples based on identical metabolite annotation and RT proximity. Peak alignment across samples was further confirmed using SpectConnect to assess similarity in spectral fragmentation patterns, and by manual curation.

Metabolomic Analyses. Metaboanalyst (v2.0) run on the statistical package R (v2.14.0) used metabolite peaks areas (as representative of concentration). These data were first analyzed by an unsupervised principal component analysis (PCA), which identified the presence of the Brg1/Brm double mutant as the principal source of variance. To sharpen the separation between our three groups, data were next analyzed using a partial least squares discriminant analysis (PLS-DA) to further determine which metabolites were responsible for separating these two groups. The specific metabolites contributing most significantly to the differences identified by PLS-DA between Brg1/Brm double mutant and control group hearts were determined using the variable importance in projection (VIP) analysis in the metaboanalyst environment. The metabolites that best differentiated the groups were then individually tested using the Student's t test (Microsoft Excel 2011, Seattle, Wash.). The t test significant metabolites were matched to metabolomics pathways using the Pathway Analysis feature in Metaboanalyst 2.0 software. Heat maps of the metabolite data (individual and grouped) were generated using the GENE-E software (broadinstitute.org/cancer/software/GENE-E/index).

Results. To determine whether there is functional compensation of Brg1 and Brm in adult cardiomyocytes, we generated Brg1 floxed/floxed mice carrying the αMHC-Cre-ERT transgene on a Brm-deficient background. To induce the Brg1 conditional mutation in cardiomyocytes, these mice were treated with oral tamoxifen by providing it in their chow for 7 days (Brg1/Brm double mutant). As a control, the same Brg1^{floxed/floxed}; αMHC-Cre-ERT^{+/0}; Brm^{-/-} mice did not receive any tamoxifen treatment (Control Group 1). This control group addressed the potential for transgene leakiness (i.e., tamoxifen-independent induction of the Brg1 mutation). As a second control, Brg1 floxed/floxed mice lacking the αMHC-Cre-ERT transgene on a Brm deficient background (Brg1^{floxed/floxed}; αMHC-Cre-ERT^{0/0}; Brm^{-/-}) were treated with tamoxifen (Control Group 2). This control group addressed the potential for tamoxifen having off-target effects unrelated to the Cre-ERT transgene. Rapid onset of cardiac dysfunction (Table 4), evidenced by significant decreases in Brg1-Brm DM mice ejection fraction % (42.0 vs. 88.6% in Control Groups 1 & 2) and factional shortening % (21.3 vs. 57.3% in Control Groups 1 & 2), led to the rapid onset of death in Brg1/Brm double mutants, but not in either control group. Kaplan-Meier analysis illustrates the first deaths occurring at day 13, with all other Brg1/Brm double mutants dying by day 22 relative to the first day of tamoxifen-fortified chow (which was provided on days 1-7). No histological changes were detected in the Brg1/Brm double mutant hearts, but increases in LV Mass/BW were identified (6.2±0.3 mg/g vs. 4.2±0.1 mg/g Brm^{-/-} flx/flx Brg1 Tg+ given 7 days of Tamoxifen at 1 day premortem, average day 11.6±1.5 days, N=27 and 30, respectively, *p\0.05 by Student's t test). The cardiac phenotype of double mutants included changes in mitochondrial dynamics and accumulation storage vacuoles based on our analysis of H&E-stained sections and TEM. Therefore, we considered the possible role of SWI/SNF complexes in regulating cardiomyocyte metabolism. To detect proximal events leading to heart failure and death, we investigated changes in the metabolomics profiles of Brg1/Brm double-mutant mice on day 9, which was 2 days after tamoxifen treatment ended. Day 9 was also a point prior to any signs of adverse health and 4 days before any of the mice were found to die.

Compared to the group 1 sibling-matched controls, quantitative non-targeted metabolomics profiling identified distinct differences in the Brm/Brg1 double mutant hearts by PCA and PLS-DA analysis. In both analyses, the principal component 1 accounted for 55% of the differences between the two groups. Further analysis of the top 15 metabolites using a VIP that differentiated the Brg1/Brm double-mutant hearts from the group 1 controls included phosphoric acid, a-monostearin, urea, glutamic acid, and lactic acid.

Compared to the group 2 sibling-matched controls, metabolomics non-targeted profiling identified distinct differences in Brg1/Brm double-mutant mice by PCA and PLS-DA analysis. Between 47.8 and 51.6% of the differences between these groups accounted for the variability described by principal component 1. Like the analysis of the Brg1/Brm double-mutant mice compared to the group 1 controls, the top 15 metabolites using a VIP analysis to differentiate Brg1/Brm double-mutant mice from group 2 controls included phosphoric acid, glutamic acid, 2-aminoadipic acid, myoinositol, creatinine, and taurine. Since these two analyses differed in ways that may have been due to the presence of tamoxifen treatment, we chose next to combine the two control groups (Groups 1 and 2) and compared them to the Brg1/Brm double-mutant mice. By PCA analysis, principal component 1 accounted for 55.5% of the variability between the two groups and by PLS-DA, principal components 1-3 accounted for 12.4, 39.6, and 20.1%, respectively and separated out the two groups distinctly. VIP analysis identified creatinine, taurine, 2-aminoadipic acid, glucose-6-phosphate, a-monostearin, and linoleic acid as the top significant hits. Comparison of the top 15 VIP hits between the three analyses performed, creatinine, linoleic acid, and glucose-6-phosphate were consistently found and represent differences not dependent upon the types of controls used (Table 5, in bold).

The comparisons of Brg1/Brm double-mutant mice to Group 1, Group 2, and Groups 1 & 2 were performed to demonstrate the need for multiple controls (i.e., how tamoxifen and genetic backgrounds influence cardiac metabolites individually). The consistent differences Brg1/Brm double-mutant mice have independent of their genotype and strain (Group 1) and Tamoxifen (Group 2) was most dependably determined when Groups 1 and 2 were combined.

Differences in Groups 1 and Group 2 were identified, as expected due to the tamoxifen treatment. For example, differences were detected by PCA, PLS-DA, and t test analysis. The differences between Groups 1 and 2 are limited, due to the low number (N=3) of biological replicates run in these subgroups. The comparison of Brg1/Brm double-mutant samples to the combined Groups 1 and 2 allowed identification of metabolic signatures that are independent of the Brg1/Brm-null phenotype.

Unsupervised heat maps illustrating the differences in metabolite quantities for each of the three individual analyses identified metabolites that were different from the controls by 5.3 to 0.35 fold. When individual VIP significant metabolites were identified in the three analyses, serine, linoleic acid, oleic acid, and taurine were >2.0 fold and fructose-6-phosphate, glucose-6-phosphate, creatinine, myoinositol were decreased <0.8 fold and compared to controls. We next took the VIP significant metabolites that were also t test significant in the three comparisons (control groupings) and performed a pathway enrichment analysis (Table 5). All three comparisons consistently identified oleic acid and linoleic acid as significant, with the combined controls additionally identifying fructose-6-phosphate, creatinine, and alanine as significant.

The significant metabolic pathways that were identified included (1) biosynthesis of unsaturated fatty acids and (2) linoleic acid metabolism; (Table 6, right column). In the context of VIP significant metabolites, increased linoleic acid may broadly involve arachidonic acid pathways. Decreased glucose-6-phophate and fructose-6-phosphate (<0.5 fold) and downstream myoinositol (decreased<0.8 fold), illustrate multiple points in which glucose metabolism is affected in amino sugar and nucleotide sugar metabolism. Similarly, evidence of alterations in alanine metabolism (increases in threonine, alanine, and serine metabolites) represents potential increases in the ability to create pyruvate through these pathways. Finally, decreased creatinine and increases in urea illustrate where reducing cardiomyocyte Brg1/Brm expression affects creatine and creatinine metabolism in vivo.

The changes in fatty acids used as energy substrates, including oleic acid in the present study, may reflect alterations in fatty acid utilization in the absence of SWI/SNF support of PPAR activity. It is also possible that SWI/SNF alters linoleic acid by its alterations in the mitochondria. Specifically, alterations in the physicochemical properties of membranes, including cardiolipins, could contribute to the cardiac phenotypes reported here. Recent studies have identified that linoleic acid preserves mitochondrial cardiolipin found in the inner membrane and attenuates mitochondrial dysfunction in heart failure. In the present study, the increases in linoleic acid may reflect this response to protect against heart failure.

In the present study, we took a non-targeted approach to metabolomically analyze the effects of inducing the simultaneous loss of Brg1 and Brm in adult cardiomyocytes at an early time point before the lethal effects were seen in vivo. Since the Brg1/Brm double-mutant model had both a genetic component and a tamoxifen feeding component, two types of controls were used and analyzed separately and together to identify significant differences in creatinine, linoleic acid, glucose-6-phosphate, oleic acid, and serine (Table 5). The increased oleic and linoleic acid in the Brg1/Brm double-mutant hearts may be linked to the role of the SWI/SNF complex in the regulation of PPARs, instrumental to the mainly fatty acid oxidation the heart uses as a primary energy source. Similarly, the decreases in cardiac G-6-P and F-6-P illustrate a dysregulation in downstream glycolysis and glycogen formation, and may reflect more complex alterations of enzymes both up and downstream.

### Example 3

### BRG1 and BRM SWI/SNF ATPases Redundantly Maintain Cardiomyocyte 3 Homeostasis by Regulating Cardiomyocyte Mitophagy and Mitochondrial Dynamics In Vivo

There has been an increasing recognition that mitochondrial perturbations play a central role in human heart failure. Mitochondrial networks, whose function is to maintain the regulation of mitochondrial biogenesis, autophagy (‘mitophagy’) and mitochondrial fusion/fission, are new potential therapeutic targets. We recently identified a role of the SWI/SNF ATP-dependent chromatin remodeling complexes in the metabolic homeostasis of the adult cardiomyocyte using cardiomyocyte-specific and inducible deletion of the SWI/SNF ATPases BRG1 and BRM in adult mice (Brg1/Brm double mutant mice). To build upon these observations in early alterated metabolism, the present study looks at the subsequent alterations in mitochondrial quality control mechanisms in the impaired adult cardiomyocyte. We identified that Brg1/Brm double-mutant mice exhibited increased mitochondrial biogenesis, increases in ‘mitophagy’, and alterations in mitochondrial fission and fusion that led to small, fragmented mitochondria.

Mechanistically, increases in the autophagy and mitophagy-regulated proteins Beclinl and Bnip3 were identified, paralleling changes seen in human heart failure. Cardiac mitochondrial dynamics were perturbed including decreased mitochondria size, reduced number, and altered expression of genes regulating fusion (Mfn1, Opa1) and fission (Drp1). We also identified cardiac protein amyloid accumulation (aggregated fibrils) during disease progression along with an increase in pre-amyloid oligomers and an upregulated unfolded protein response including increased GRP78, CHOP, and IRE-1 signaling. Together, these findings described a role for BRG1 and BRM in mitochondrial quality control, by regulating mitochondrial number, mitophagy, and mitochondrial dynamics not previously recognized in the adult cardiomyocyte. As epigenetic mechanisms are critical to the pathogenesis of heart failure, these novel pathways identified indicate that SWI/SNF chromatin remodeling functions are closely linked to mitochondrial quality control mechanisms.

As heart failure treatment has evolved, increasing recognition of mitochondria's role in the pathogenesis of disease has emerged. New techniques allow the discovery of mitochondrial networks, whose function is maintained by three processes, including (1) mitochondrial biogenesis (increase in mitochondrial number), (2) mitophagy, and (3) continuous mitochondrial fission (division) and fusion. Impaired mitochondrial biogenesis is a feature of myocardial hypertrophy and end-stage ischemic heart failure in humans, while autophagy has been central to defects in human heart failure. Evidence of altered mitochondrial dynamics (fission and fusion) in human heart failure, specifically in idiopathic dilated cardiomyopathy, has recently been reported.

Changes in the mitochondrial network influence the production of ROS and ATP production, most notably offset by activating mitochondrial biogenesis and mitophagy in exercise and other therapeutic modalities. Complementing mitochondrial biogenesis is the ongoing process of “mitophagy” (mitochondrial autophagy), where damaged/dysfunctional mitochondria are targeted for destruction via lysosomal degradation. Here mitochondrial proteins such as BNIP3L and BNIP3 are abundant in the heart and act as targeting molecules that attract autophagosome to mitochondria and are critical to maintaining cardiac function. Lastly, regulation of mitochondrial dynamics (fission and fusion) involves the redistribution of mitochondrial content. With the enormous continuous ATP demand of the cardiomyocyte, the importance of maintaining mitochondrial quality control is of particular significance in cardiac disease, including heart failure, ischemic heart disease/myocardial infarction, and diabetes.

Epigenetic regulation of heart failure occurs by several key mechanisms, including ATP-dependent chromatin remodeling. One ATP-dependent chromatin remodeling complex in cardiomyocytes are the SWI/SNF (SWItch/Sucrose Non-Fermentable) complexes, composed of 9-12 subunits, including one of two ATPases critical to their function (1) BRG1 (Brahma-related gene 1) or (2) BRM(Brahma). Recent studies demonstrate a role of BRG1 and BRM in maintaining homeostasis in the adult cardiomyocyte. These studies identified that cardiomyocyte-specific and inducible deletion of BRG1 and BRM in adult mice (Brg1/Brm double mutant mice), alterations in glucose-6-phosphate, fructose-6-phosphate, and myoinositol at day 10 post-induction occurred before severe cardiomyopathy is seen. The identification of these early metabolic changes led to the current study to identify the underlying mechanisms by which BRG1 and BRM deletion affects mitochondrial quality control mechanisms in the impaired adult cardiomyocyte.

In the present study, we build upon our findings that Brg1/Brm double mutant hearts have early metabolic changes at Day 10 by investigating the changes occurring 15 days after inducing Brg1 deletion, just before the development of a severe cardiomyopathy. We identified alterations in mitochondrial biogenesis, mitochondrial autophagy (mitophagy), and mitochondrial fission and fusion just before the death that occurred in all mice by Day 22. We demonstrate a previously undescribed link between the chromatin remodeling SWI/SNF components BRG1 and BRM with mitochondrial homeostasis.

Animal creation and experimental design. The αMHC-Cre-ERT mice were obtained from The Jackson Laboratory (#005,657, Bar Harbor, Me.); the Brg1 conditional mutant mouse line and Brm constitutive mutant mouse line have been described previously. Genotyping of the αMHC-MerCreMer, Brg1 foxed, and Δfloxed alleles and the Brm mutation was done by PCR.

Histology, transmission electron microscopy (TEM), and image analysis.

Immunohistochemistry (IHC) was performed on a fixed histological section using an anti-BRG1 rabbit polyclonal antibody (Millipore 124 #07-478, Temecula, Calif., USA), anti-GRP78 mouse monoclonal antibody, anti-CHOP (GADD153), or phospho-eIF2. Hearts analyzed by TEM were viewed on a LEO EM910 transmission electron microscope operating at 80 kV (LEO Electron Microscopy, Thornwood, N.Y., USA). An average of 2500 mitochondria were analyzed from 30 fields from multiple levels from three hearts per mouse cohort.

Analysis of autophagic flux in vivo. Autophagic flux was determined in vivo from the titration of bafilomycin A1. Antibodies for LC3B isoforms (Sigma-Aldrich #L7543) and beclin were used for Western immunoblot analysis of markers of autophagy (Cell Signaling #3495P, 136 Beverly, Mass., USA).

Quantitative analysis of cardiac amyloid (protein aggregation) and pre-amyloid oligomers (PAOs). Protein aggregation was assessed using an Enzo Proteostat Protein Aggregation Assay (#ENZ-51,023, Farmingdale, N.Y., USA) in conjunction with their pre-formulated aggregation standards (#ENZ-51,039), and pre-amyloid oligomer staining was performed.

RNA isolation and RT-qPCR analysis. Total RNA was isolated from cardiac tissue homogenized with a TissueLyser LT (Qiagen N.V. #69,980, Venlo, The Netherlands) using Trizol (Life Technologies #15,596-026, Carlsbad, Calif., USA) according to the manufacturer's instructions. TaqMan gene expression assays (Life Technologies) were performed using universal TaqMan master mix (Life Technologies #4,304,437) commercial probes.

Analysis of mitochondrial DNA by qPCR. Mitochondrial number was quantified by qPCR on DNA prepared from whole-heart homogenates using the DNAeasy Blood and Tissue Kit (Qiagen #69,506). SYBR Green and primers for the mitochondrial DNA included mtNd1, mtCO1, and mtCytb1; H19 was run in parallel as a nuclear DNA control.

Statistics. SigmaPlot (Systat Software, San Jose, Calif., USA) was used to determine significant statistical difference by one-way ANOVA followed by post hoc analysis using the Holm-Sidak method or a Student's t test, as indicated. Pb.05 was considered significant.

A mouse model reveals SWI/SNF complexes are essential in adult cardiomyocytes. To investigate the combined role of BRG1 and BRM in adult cardiomyocytes, we crossed mice carrying an inducible, cardiomyocyte-specific αMHC-Cre-ERT transgene to Brg1^{fl/fl}; Brm^{-/-} mice. Adult Brg1^{fl/fl}; αMHC-Cre-ERT^{+/0}; Brm^{-/-} mice were then fed tamoxifen-fortified chow for 7 days to induce the Brg1 floxed-to-Δfloxed excision event, which was confirmed by both PCR and IHC. These mice (herein referred to as Brg1/Brm double mutants) were monitored by echocardiography until they died, which occurred within 22 days of initiating the tamoxifen diet. A progressive heart failure occurred prior to death in Brg1/Brm double-mutant but not in control mice (Table 7). Six groups of control mice were analyzed in parallel to ensure that we were observing a Brg1/Brm double-mutant phenotype rather than an artifact, such as tamoxifen itself or tamoxifen-induced Cre dysfunction.

Brg1/Brm double-mutant cardiomyocytes undergo mitophagy and altered mitochondrial dynamics. Considering the severity of the cardiac phenotype and the rapid lethality of mice lacking Brg1 and Brm in their cardiomyocytes, the histopathology was surprisingly mild. Analysis of H&E- and Mason's Trichrome-stained sections revealed that the Brg1/Brm double-mutant phenotype ranged from relatively normal to moderate vacuolization with no evidence of lipid accumulation by oil red 0 staining and with fibrosis increased only in rare individuals.

Next, we assessed fetal cardiomyocyte gene expression because it is a typical pathological hypertrophic response that occurs during heart failure. Skeletal muscle actin was significantly decreased at both time points in the Brg1/Brm double-mutant hearts compared with controls. PMHC fetal gene expression was significantly elevated at day 10 post-tamoxifen induction (early time point) but not at 1-day pre-mortem (late time point). In contrast, Bnp and Anf mRNA were not changed significantly at either time point. These data are not representative of a typical pathologic hypertrophy/heart failure response. Ultrastructural analysis of the Brg1/Brm double-mutant heart by TEM was distinct by the presence of alterations limited to the interfibrillar areas (mitochondrial compartment).

In all of the Brg1/Brm double-mutant hearts, we identified mitochondria degeneration and an increase in double-membrane bound vacuoles with mitochondrial remnants within that were absent in all of the parallel control hearts. Ultrastructurally, these vacuoles appeared similar to those found to be increased in the process of autophagy, specifically autophagosomes. Autophagy is a lysosomal degradation pathway for cytoplasmic material and organelles, starting at the endoplasmic reticulum-mitochondria interface. The conserved autophagic process shuttles cytoplasmic components to fuse with lysosomes. This stepwise engulfment of cytoplasmic material by the phagophore, maturing into a double-membrane-bound vesicle, forms the autophagosome. To confirm that Brg1/Brm double-mutant hearts had quantitative alterations in autophagy, we next analyzed autophagic flux in vivo.

The role of autophagy in cardiovascular biology has proven to be context dependent, widely characterized in cardiomyocytes, and critical to the maintenance of cardiovascular homeostasis and function. Depending on context, there is a window of optimal autophagic activity, whereby increases or decreases may be protective depending upon the cardiac stress disease state. Brg1/Brm double-mutant hearts exhibited a significant increase in autophagic flux, as illustrated by the LC3II:LC3I ratio by Western immunoblot after bafilomycin A1 treatment, compared with control mice run in parallel. In parallel with this evidence of increased autaphagic flux, we identified increased Beclin 1 by Western immunoblot analysis. With evidence of increased autophagy, we next investigated mechanisms responsible for upregulating autophagy.

Central to the regulation of autophagy in heart disease, the Beclin 1 protein functions as a scaffolding protein assembling Beclin 1 interactome to regulate Class II PI3K/VPS34 activity, which tightly controls autophagy at multiple stages. The Brg1/Brm double-mutant mice had significantly increased Beclin 1 protein expression compared with control group mice. Autophagy is also regulated at the transcriptional level, with increased Atg5, Atg7, Atg12, Bnip3, and Vps34 mRNA to enhance autophagy. RT-qPCR analysis of these genes demonstrated the Brg1/Brm double-mutant hearts had a significant increase in Bnip3 mRNA levels, while Atg12 and Vps34 were significantly decreased compared with control hearts at day 15. At the earlier day 9 time point, Brg1/Brm double-mutant hearts had increased Vps34 mRNA and decreased Bnip3 mRNA, illustrating the dynamic expression of autophagy regulators. The transcriptional and epigenetic network regulating autophagy is emerging, which underscores the diverse ways in which autophagy can be regulated depending on the stimulus.

To test whether BRG1 and BRM occupy the Bnip3 promoter, we performed quantitative ChIP assays. Significant enrichment of BRG1 and BRM was detected in cardiac tissue, which strongly suggests that BRG1- and BRM-catalyzed SWI/SNF complexes directly regulate Bnip3. BNIP3, primarily located in the mitochondria as an integrated protein, perturbs outer membrane integrity, induces permeabilization of the inner mitochondrial membrane, and can stimulate mitophagy directly by triggering depolarization and Parkin recruitment. Taken together, these findings illustrate a novel role for BRG1 and BRM in the maintenance of cardiomyocyte autophagic flux, specifically related to the mitophagy as illustrated by the TEM analysis and the direct regulation of Bnip3.

BNIP3 alters cellular mitochondrial dynamics, a continuous process by which mitochondria fuse together (fusion) and subsequently divide (fission) to maintain their functional balance. Specifically, increasing BNIP3 expression can induce mitochondrial fragmentation (fission) in cells. Understanding this link led us to evaluate further the Brg1/Brm double-mutant heart mitochondrial phenotype in our TEM studies. Here TEM revealed an apparent increase in Brg1/Brm double-mutant heart mitochondrial fragmentation compared with controls. Analysis of mitochondria over multiple LV regions revealed that the number of small mitochondria identified in the Brg1/Brm double-mutant hearts was skewed with a marked increase in small mitochondria with significantly smaller areas.

To put in context the apparent increase in small mitochondria, we determined mitochondrial number by qPCR of three mitochondrial encoded genes normalized to the nuclear-encoded H19 and found a significant decrease in the number of mitochondria. The fusion and fission of mitochondria is a dynamic process that cells use for responding to mitophagy and maintaining protein quality. The regulation of mitochondrial dynamics by fusion and fission is regulated by four dynamin-related GTPases, including DRP1 and FIS1 (fission) and mitofusins (e.g., MFN1) in the outer membrane and OPA1 in the inner membrane (fusion). RT-qPCR analysis of these GTPases in the Brg1/Brm double-mutant hearts demonstrated significant decreases in Mfn1, Opa1, and Drp1 in late disease but not at an earlier stage. These findings demonstrate the interesting juxtaposition of increased mitophagy and mitochondrial fission, which has been shown to allow the selective elimination of damaged mitochondria by autophagy.

Activation of the UPR in Brg1/Brm double-mutant cardiomyocytes. Histological analysis of the Brg1/Brm double-mutant hearts stained with H&E revealed stereotypical pink accumulation inside of the cardiomyocytes, resembling the unfolded proteins that accumulate in cardiac amyloidosis. We next investigated the presence of unfolded proteins using a colorimetric assay that has been used to identify amyloid and other unfolded proteins in experimental disease processes. At an early time point, Brg1/Brm double-mutant hearts did not have an increase in unfolded proteins. However, at a later time point when mitophagy and altered fission and fusion were present, a significant increase in unfolded proteins was present with a ˜3-fold increase compared with control hearts.

Using the All antibody, we investigated the presence of pre-amyloid oligomers (PAOs) in early and late Brg1/Brm double-mutant heart samples. The detection of low levels of PAO by the All antibody present in cardiomyocytes detected by an antibody against the heavy chain myosin II using immunofluorescence indicates that soluble PAOs were increasing at day 15 in Brg1/Brm double-mutant hearts. While not reaching significant levels, these findings suggest that the protein aggregates detected in the heart at day 15 go through a PAO state very quickly to accumulate amyloid in significant amounts when Brg1 and Brm have been ablated in adult cardiomyocyte in vivo.

Having established the presence of increased amyloid in these hearts, we next investigated cardiac expression of GRP78 by IHC and identified that Brg1/Brm double-mutant mice had markedly increased GRP78 expression compared with controls at the late time point where PAOs and amyloid were increased.

Identification of the spliced Xbp-1 by RT-qPCR is one way in which IRE-1 activity is measured. We identified that Brg1/Brm double-mutant hearts had a significant increase in the spliced Xbp-1 mRNA compared with control hearts. Downstream of the IRE1-XBP-1 pathway, XBP1 binds the promoter of the C/EBPα (Cebpa) gene to increase its expression. Brg1/Brm double-mutant mice had significantly more Cebpa mRNA expression compared with controls. The significant increase in Brg1/Brm double-mutant heart Xbp-1 splice variant mRNA and Cebpa mRNA demonstrate the increased activity via IRE-1 signaling. The relationship between XBP1 and C/EBPβ (Cebpb) is a bit more complex, with evidence that C/EBPβ induces expression of XBP1 protein. Brg1/Brm double-mutant hearts had diminished Cebpb mRNA levels that were not significant.

The stress caused in the ER by the presence of unfolded proteins can also cause signaling through PERK, whereby PERK proteins dimerize and undergo autophosphorylation. This autophosphorylation leads to downstream phosphorylation of eIF2, which we investigated by immunohistochemistry (IHC). Here we found evidence that the Brg1/Brm double-mutant mice had a higher cardiac eIF2 expression histologically, and next looked at downstream ATF4 and CHOP expression by RT qPCR. Significant increases in both Chop and Atf3 mRNA were identified by RT qPCR in the Brg1/Brm double-mutant mice, with increases in CHOP protein also seen by IHC in the Brg1/Brm double-mutant mice. Activation of ATF4 increases target gene expression, including the transcription factor CHOP (CCAAT/-enhancer-binding protein homologous protein), which itself induces the expression of Atf3 mRNA. While the lack of reliable reagents prevented us from directly measuring ATF6 signaling, we determined the mRNA levels of other reported genes involved in the UPR. We found that Brg1/Brm double-mutant hearts had either significant down-regulation of Ire-1a, Atf6a, and Grp78 reflecting compensatory responses to the clear GRP78 and IRE-1 signaling present, or no change in genes not as clearly related to the UPR in cardiomyocytes.

This study identifies a novel role of the SWI/SNF components BRG1 and BRM in the homeostasis of the adult cardiomyocyte regulating mitochondrial biogenesis, mitophagy, and mitochondrial fission and fusion. While there have been suggestions that SWI/SNF complexes may be related to mitochondrial biogenesis and autophagy in mammalian cells, the present study is the first to link them additionally to mitochondrial fission and fusion. While these findings parallel changes seen in heart failure phenotypes, BRG1 and BRM have previously been shown to have a role in the development of pathological cardiac hypertrophy. BRG1 interactions with the DNA-binding protein PARP1 have been implicated in the prototypical shift in cardiac MHC isoform expression in pathological cardiac hypertrophy, and may explain the decreases (instead of increases) of βMHC seen with the heart failure in the present study.

In the present study, we describe the redundant role of BRG1 and BRM to maintain the adult cardiomyocyte in vivo. Notably, multiple distinct alterations have been identified in this model, with early metabolomics changes previously identified, accompanying the upregulation of autophagy/mitophagy, altered mitochondrial dynamics, accumulation of PAOs/aggregated protein, and activation of the UPR that culminated in severe bradycardia and death.

The increased autophagic flux present in Brg1/Brm double-mutant hearts was characterized morphologically by increases in mitochondria in what look like autophagosomes and by increased Beclin 1 protein and Bnip3 mRNA. We demonstrate that BRG1 and BRM occupy the Bnip3 promoter and regulate its expression as well as autophagic flux. The transcriptional activation of Bnip3, as seen in the Brg1/Brm double-mutant hearts, has been reported to be by C/EBPβ, E2F1, FOXO1/3, HIF1, and STAT3. The enhanced Beclin 1, also seen in the Brg1/Brm double-mutant hearts, has been reported to be regulated by FOXO1/3, NF-ϰB, p63, and Stat. BNIP3 is a receptor, like PINK1 and Parkin, that regulates mitochondrial autophagy (also known as mitophagy), consistent with the morphological findings in Brg1/Brm double-mutant hearts. Increases in BNIP3 expression occur after myocardial infarction and in response to chronic pressure overload hypertrophy and may be a mechanism by which mitochondrial pruning is mediated to get rid of damaged mitochondria during increased stress. BRG1 and BRM may be linked to the regulation of HIF1, as the SWI/SNF complex regulates HIF-1-dependent mitophagy (regulated through BNIP3 and Beclinl) as a metabolic adaptation to stress in cancer cells.

The regulation of mitochondrial autophagy and mitochondrial dynamics is logically linked to mitochondrial quality control. Mitochondria are damaged by many environmental stressors, and repair is dependent upon the mitochondrial fission and fusion (broadly known as mitochondrial dynamics) and autophagy. In fact, mitochondrial fusion and fission are processes essential for the preservation of normal mitochondrial function. In heart failure, OPA1 is important for maintaining normal cristae structure and function, while preserving the inner membrane structure and for protection against apoptosis. Confocal and TEM analysis have demonstrated that failing hearts have small, fragmented mitochondria, consistent with decreased fusion. Reducing OPA1 or MFN1 in cardiomyocytes results in increased mitochondrial fragmentation. The transcriptional regulation of genes involved with mitochondrial fusion in cardiomyocytes (i.e., OPA1 and MFN1) as well as fission (e.g., DRP1) has been recently shown to be under the control of PGC-1α by co-activating the estrogen-related receptor a. While the SWI/SNF complex component BAF60a supports PGC-1α co-activation, the regulation of mitochondrial fission by Brg1/Brm demonstrated here has not been described previously.

Animal creation and experimental design. To induce the Brg1 conditional mutation in adult cardiomyocytes, 3-6 month old male and female mice were provided rodent chow containing tamoxifen (Sigma-Aldrich #T5648, St. Louis, Mo.) over a 7-day period. The route of delivery and dose were selected to minimize a previously described artifact caused by high doses of tamoxifen in the presence of the MHC-Cre-ERT transgene1. Briefly, 500 mg of tamoxifen was mixed with 1 kg of ground-up rodent chow and then mixed with water, kneaded into pellets, and dried in a hood. Provided to mice ad libitum, the dose was estimated to be 80 mg/kg/day. After the 7-day treatment period, the tamoxifen-fortified chow was removed and replaced with the same chow lacking tamoxifen.

Histology, transmission electron microscopy (TEM), and image analysis. Histology was performed by fixing adult heart tissues in 4% paraformaldehyde or 10% formalin, embedding in paraffin, and cutting 5-μm sections according to standard procedures. Sections were either stained with hematoxylin and eosin (H&E), Mason's Trichrome, or processed for immunohistochemistry (IHC) using an anti-BRG1 rabbit polyclonal antibody (Millipore #07-478, Temecula, Calif.), anti-GRP78 mouse monoclonal antibody2, anti-CHOP (GADD153), or phospho-eIF2. Imaging of stained sections was obtained using Aperio Scanscope and Aperio Imagescope software (version 10.0.36.1805, Aperio Technologies, Inc., Vista, Calif.). Fibrosis was determined using the Aperio Imagescope's Positive Pixel Count Algorithm to analyze Masson's Trichrome stained 4-chamber sections, Hue Value=0.66 (blue), Hue Width=0.1 (detection threshold above background white). The pen tool was used to isolate tissue sections to analyze and the percent fibrosis was expressed as the weighted average percent of the N positive (collagen blue)/N total (tissue, defined by the non-white area). For transmission electron microscopy (TEM), hearts were fixed in 2% paraformaldehyde and 2.5% glutaraldehyde in 0.15M sodium phosphate buffer (pH 7.4), overnight and then post-fixed with 1% osmium tetroxide/0.15M sodium phosphate buffer. Samples were dehydrated with increasing concentrations of ethanol, infiltrated, and embedded in Polybed 812 epoxy resin (Polysciences, Warrington, Pa.). One-micron sections were prepared to select representative areas by light microscopy, and 70-nm ultrathin sections were cut with a diamond knife. Sections were mounted on 200 mesh copper grids and stained with 4% aqueous uranyl acetate and Reynolds' lead citrate. Sections were observed with a LEO EM910 transmission electron microscope operating at 80 kV (LEO Electron Microscopy, Thornwood, N.Y.) and photographed with a Gatan Orius SC1000 CCD Digital Camera and Digital Micrograph 3.11.0 (Gatan, Pleasanton, Calif.). Mitochondria cross-sectional areas were measured using NIH ImageJ at magnifications of 5,000-10,000×. The global scale was set according to the image specific scale generated by Gatan camera output. An average of 2,500 mitochondria were analyzed from 30 fields from multiple levels from three hearts per mouse cohort.

Analysis of autophagic flux in vivo. Autophagic flux was determined in vivo from the titration of bafilomycin A1. Briefly, stock bafilomycin A1 (BFA, Santa Cruz Biotechnology #201550A, Santa Cruz, Calif.; 3 mM stock in dimethyl sulfoxide) diluted in phosphate-buffered saline (40% dimethyl sulfoxide final) was injected intraperitoneally to a final concentration of 3 mmol/kg in 200 μL and repeated one hour later. One hour after the second injection, mice were euthanized and heart tissues were collected for analysis by Western immunoblot for LC3B isoforms (Sigma-Aldrich #L7543). The Beclin autophagy marker was also analyzed by Western immunoblot (Cell Signaling #3495P, Beverly, Mass.).

Quantitative analysis of cardiac amyloid (protein aggregation) and pre-amyloid oligomers (PAOs). Protein aggregation was assessed using an Enzo Proteostat Protein Aggregation Assay (#ENZ-51023, Farmingdale, N.Y.) in conjunction with their preformulated aggregation standards (#ENZ-51039). Briefly, protein lysates were prepared from flash-frozen apical cardiac tissues using 8M urea lysis buffer, protein concentrations were determined by Bradford assays, and assays were performed in 96-well, blackwalled, clear-bottom plates read on a Clariostar microplate reader at 550 nm excitation and 600 nm emission with gain setting between 1000-2000. Pre-amyloid oligomer staining was performed.

RNA isolation and RT-qPCR analysis. Cardiac tissues were homogenized using a TissueLyser LT (Qiagen N.V. #69980, Venlo, The Netherlands) according to the manufacturer's protocols. Approximately 20-40 mg of apical ventricle was homogenized in 1 mL of Trizol (Life Technologies #15596-026, Carlsbad, Calif.) using a 5-mm stainless steel bead (Qiagen N.V. #69989). Chloroform (2004) was added, centrifuged at 12,000 g (15 min at 4° C.), isopropanol (0.5 mL) was then added to the aqueous phase, centrifuged at 12,000 g (10 min at 4° C.), and the resulting RNA pellet was washed with 1 mL of 75% ethanol, centrifuged at 7500 g (5 min at 4° C.). The resulting pellet was dried and resuspended in RNase-free water. RNA concentrations were then determined by UV spectroscopy (absorbance of 260-280 nm). RNAs (500 ng) were reverse-transcribed using iScript reverse transcription supermix (Bio-Rad Laboratories #170-8841, Hercules, Calif.). TaqMan gene expression assays (Life Technologies) were performed using universal TaqMan master mix (Life Technologies #4304437) to measure markers of hypertrophy and fission/fusion. Cardiac hypertrophy fetal gene expression was monitored using probes for β-Mhc (Mm00600555_m1), skeletal muscle α-actin (Mm00808218_g1), Anf (Mm01255747_g1), and Bnp (Mm00435304_g1) mRNAs. Fission and fusion gene expression was quantified using probes for Opa1 (Mm00453879_m1), Drp1 (Mm01342903_m1), Fis1 (Mm00481580_m1), and Mfn1 (Mm00612599_m1) mRNAs. Primer pairs for other RT-qPCR assays are listed in Table 8.

For gene expression calculations, the threshold value at Cross point (Cp) was determined for each target when the number of cycles at which the fluorescence rose appreciably above the background fluorescence. Relative target gene expression levels were first normalized to BestKeeper Index and then normalized to control groups. Fold change values (calculated by the formula 2 (−ΔΔCq) were used in the analysis. To select suitable reference genes in qPCR for normalization, the stability of the candidate gene expression was statistically analyzed with a Microsoft Excel-based software: BestKeeper applet. BestKeeper estimated the gene expression variation for reference by the calculated standard deviation (SD) and coefficient of variation (CV) using their raw Cp values derived from qPCR as input.

The candidate genes with SD values lower than 1 were considered to be stably expressed. Five housekeeping genes were analyzed, including Gapdh (NM_001289726.1, UPL probe #80 #689038, primers: tgtccgtcgtggatctgac (SEQ ID NO:1), cctgcttcaccaccttcttg, SEQ ID NO:2), Atcb (NM_007393.3, UPL probe #64 #688635, primers: ctaaggccaaccgtgaaaag (SEQ ID NO:3), accagaggcatacagggaca, SEQ ID NO:4), Pgk (NM_008828.2, UPL probe #108 #692276, primers: tacctgctggctggatgg (SEQ ID NO:5), cacagcctcggcatatttct, SEQ ID NO:6), and G6PHD (NM_008062.2, UPL probe #76, #688996, primers: ttaaatgggccagcgaag (SEQ ID NO:7), tgctctgccatgatgttttc, SEQ ID NO:8), using the Roche universal probe system. The most stably expressed genes were determined based on the Pearson correlation coefficient (r) and the BestKeeper index (BI), which were the geometric means of Cp values of these candidate reference genes, and the corresponding p values. The Bestkeeper applet was used to calculate the gene expression variation of reference genes. This program was designed based on the input from each reference's Cp values. Reference gene fitness was determined by measuring a panel of the following genes: Gapdh, 18S, β-actin, Pgk1, and P6PDH. Based on the statistical data for maximal stability analysis7, three reference genes, Gapdh, 18S, and Pgk1, were selected. The descriptive statistical SD, CV, correlation coefficient (r), and corresponding P values of the 3 selected reference genes are shown in Table 8.

Commercial probes used included fl-Mhc (Mm00600555_m1), skeletal muscle α-actin (Mm00808218_g1), Anf (Mm01255747_g1), Bnp (Mm00435304_g1) Opa1 (Mm00453879_m1), Drp1 (Mm01342903_m1), Fis1 (Mm00481580_m1), and Mfn1 (Mm00612599_m1) mRNA. Primer pairs for other RT-qPCR assays are listed in Table 8.

Based on the statistical data for maximal stability analysis, three reference genes, Gapdh, 18S, and Pgk1, were selected. The descriptive statistical SD, CV, correlation coefficient (r), and corresponding P values of the 3 selected reference genes are shown in Table 8.

Analysis of mitochondrial DNA by qPCR. Mitochondrial number was quantified by qPCR on DNA prepared from whole-heart homogenates using the DNAeasy Blood and Tissue Kit (Qiagen #69506). SYBR Green and primers for the mitochondrial DNA included mtNd1, mtCO1, and mtCytb1; H19 was run in parallel as a nuclear DNA control.

### Example 4

### Upregulation of Autophagy Genes and the Unfolded Protein Response in Human Heart Failure Patients

The cellular environment of the mammalian heart is constantly challenged with environmental and intrinsic pathological insults, which affect the proper folding of proteins in human heart failure. The effects of damaged or misfolded proteins on the cell can be profound and results in a process termed “proteotoxicity.” While proteotoxicity is best known for its role in mediating the pathogenesis of neurodegenerative diseases such as Alzheimer's disease, it's role in human heart failure has been recognized. The UPR involves three branches, including PERK, ATF6, and 3) IRE1. In the presence of misfolded proteins, the GRP78 molecular chaperone that normally interacts with PERK, ATF6, and IRE-1 in the endoplasmic reticulum detaches to attempt to stabilize the protein. Removing GRP78 from these internal receptors activates PERK, ATF6, and IRE-1 signaling, with mouse models of cardiac hypertrophy, ischemia, and heart failure demonstrating increases in all three. Recent studies have linked elevated PERK and CHOP in vitro with regulation of ion channels linked with systolic human heart failure. With this in mind, we specifically investigated 10 patients with arrhythmias, including left and right bundle branch blocks, atrial fibrillation, non sustained/paroxysmal/polymorphic ventricular tachycardia, and bradycardia for expression of UPR genes compared to non-heart failure controls. We identified elevated Chop, Atf3, and Grp78 mRNA, along with XBP-1-regulated cebpa mRNA, indicative of activation of the UPR in human heart failure with arrhythmias.

The cellular environment of the mammalian heart is constantly challenged with environmental and intrinsic pathological insults, which affect the proper folding of proteins in human heart failure. The effects of damaged or misfolded proteins on the cell can be profound and results in a process termed “proteotoxicity”. While proteotoxicity is best known for its role in mediating the pathogenesis of neurodegenerative diseases such as Alzheimer's disease, it's role in human heart failure has been recognized. Attempts at reversing the accumulation of misfolded proteins by exercise or induction of autophagy directly experimentally attenuate both the clearance of soluble misfolded pre-amyloid oligomers and the heart failure it induces.

The unfolded protein response (UPR) has been studied in diabetes and neurological diseases as a response to the presence and/or accumulation of damaged proteins in the endoplasmic reticulum. The UPR stress response is highly conserved from yeast to mammalian cells and has a protective role in cell survival by eliminating misfolded proteins; when prolonged, however, the UPR can induce cell dysfunction and death. In the heart, the UPR is not well understood and has been implicated in participating in protecting and impairing heart function in experimental system. Sustained overactivation of the UPR has been implicated in prion diseases as well as several other neurodegenerative diseases, and inhibiting the UPR (e.g., by the methods of this invention) could become a treatment for those diseases. Diseases amenable to UPR inhibition include Creutzfeldt-Jakob disease, Alzheimer's disease, Parkinson's disease, and Huntington's disease.

The UPR involves three branches, each with distinct signaling pathways to enhance protein folding or attenuate protein synthesis and loading of the endoplasmic reticulum 1) PERK; 2) ATF6, and 3) IRE1. In the presence of misfolded proteins, the GRP78 molecular chaperone that normally interacts with PERK, ATF6, and IRE-1 in the endoplasmic reticulum detaches to attempt to stabilize the protein. By removing GRP78 from these internal receptors, activation of PERK, ATF6, and IRE-1 occurs.

We investigated 10 patients with arrhythmias, including left and right bundle branch blocks, atrial fibrillation, non-sustained/paroxysmal/polymorphic ventricular tachycardia, and bradycardia for expression of autophagy and UPR genes compared to non-failing heart controls.

Human heart failure samples. Human samples are from subjects consented and collected for future research by the Duke Human Heart Repository (Pro00005621). Samples were de-identified without PHI and collected from heart transplant donors. De-identified heart samples were chosen based on the presence of cardiac arrhythmia, which included prolonged QT, right or left bundle branch block, ventricular tachycardias, and other non-specified dysrhythmias, as indicated in Table 10.

RNA isolation and RT qPCR analysis of mRNA. Cardiac tissues were homogenized using a TissueLyser LT (Qiagen N.V. #69980, Venlo, The Netherlands) according to the manufacturer's protocols. Approximately 20-40 mg of apical ventricle was homogenized in 1 mL of Trizol (Life Technologies #15596-026, Carlsbad, Calif.) using a 5-mm stainless steel bead (Qiagen N.V. #69989). Chloroform (200 μL) was added, centrifuged at 12,000 g (15 min at 4° C.), isopropanol (0.5 mL) was then added to the aqueous phase, centrifuged at 12,000 g (10 min at 4° C.), and the resulting RNA pellet was washed with 1 mL of 75% ethanol, centrifuged at 7500 g (5 min at 4° C.). The resulting pellet was dried and resuspended in RNase-free water. RNA concentrations were then determined by UV spectroscopy (absorbance of 260-280 nm). RNAs (500 ng) were reverse-transcribed using iScript reverse transcription supermix (Bio-Rad Laboratories #170-8841, Hercules, Calif.). TaqMan gene expression assays were performed using universal TaqMan master mix (Life Technologies #4304437) using the fourteen primer pairs shown in Table 9.

Statistical analysis. SigmaPlot (Systat Software, Inc., San Jose, Calif.) was used to determine significant statistical difference by Student's t-test. A p value<0.05 was considered significant.

The unfolded protein response (UPR) includes signaling by IRE-1, PERK, and ATF6, when misfolded protein displaces GRP78 from these receptors in the endoplasmic reticulum. We investigated how IRE-1 signaling was affected by measuring spliced Xbp-1 and the XBP-1 regulated expression of the C/EBP (Cebpa) gene expression. Compared to non-failing human heart controls, the human heart failure patients did not have increased spliced Xbp-1 mRNA compared with control hearts was seen. However, significant increases in hCebpa mRNA were identified, consistent with increased IRE-1 pathway signaling. The relationship between XBP1 and C/EBPb (Cebpb) is a bit more complex, with evidence that C/EBPb induces expression of XBP1 protein. The heart failure samples did not demonstrate any differences in hCebpb mRNA from controls.

The stress caused in the ER by the presence of unfolded proteins can also cause signaling through PERK, whereby PERK proteins dimerize and undergo autophosphorylation. This autophosphorylation leads to downstream phosphorylation of eIF2 and downstream elevations in CHOP expression by RT qPCR. We identified significant increases in both Chop and Atf3 mRNA in the human heart failure samples. Activation of ATF4 increases target gene expression, including the transcription factor CHOP (CCAAT/-enhancer-binding protein homologous protein), which itself induces the expression of Atf3 mRNA. While activation of ATF6 signaling was not directly measured, due largely to the deficiencies in reliable reagents, we determined the mRNA levels of other reported genes involved in the UPR. We identified significant increases in Grp78, reflecting compensatory responses to the clear GRP78 and IRE-1 signaling present, or no change in genes not as clearly related to the UPR in cardiomyocytes. In summary, the significant increases in Cebpa, Chop, Atf3, and Grp78 mRNA seen in human heart failure provided evidence for activation of the IRE-1 and PERK signaling pathways.

In neurodegenerative diseases (e.g., Alzheimer's disease) increases in autophagy ameliorate ER stress and the UPR by eliminating misfolded proteins. We investigated the transcriptional regulation of autophagy in these human heart failure hearts. Central to the regulation of autophagy in heart disease, the Beclin 1 protein functions as a scaffolding protein assembling Beclin interactome to regulate Class II PI3K/VPS34 activity, which tightly controls autophagy at multiple stages. Autophagy is also regulated at the transcriptional level, with increased Atg5, Atg7, Atg12, Bnip3, and Vps34 mRNA to enhance autophagy. RT-qPCR analysis of these genes in human heart failure identified significantly increased Beclinl and Vps34 mRNA, along with increased Atg5 and Atg7 mRNA, consistent with increased autophagy.

We interrogated heart failure patients with arrhythmias to determine activation of the UPR in the current study. We identified significant increases in Cebpa, Chop, Atf3, and Grp78 mRNA in human heart failure providing evidence for activation of the IRE-1 and PERK signaling pathways. In addition, we identified evidence for increases in autophagy as well in these patients.

### Example 5

### A Regulatory Mechanism Linking SWI/SNF Chromatin-Remodeling Complexes to Cardiomyocyte Conduction Prevents Cardiomyopathy and Sudden Death

Heart failure remains the most common cause of death and disability in the world. Despite vast improvements in outcomes, current therapies targeting the neurohormonal system have limitations, and additional therapies targeting other mechanisms directly leading to death are needed. Switching defective/sucrose non-fermenting (SWI/SNF) complexes play a critical role in chromatin remodeling, including the BRG1 (brahma-related gene 1) and BRM (brahma) catalytic subunits with ATPase activity. BRG1 and BRM mutations result in syndromic congenital heart defects, including Coffin-Siris Syndrome and Nicolaides-Baraitser Syndrome, consistent with their role in supporting critical developmental transcription factors (e.g., TBS5, GATA4). However, the role of BRG1 and BRM in the adult cardiomyocyte has not been identified, nor has their role in the pathophysiology of heart failure. In the present study, we identify for the first time the role of cardiomyocyte BRG1 and BRM in inhibiting c-Myc transcription, mechanistically linking the development of lethal arrhythmias in vivo, including human heart failure patients. Since BRG1 and BRM are regulated by post-translational modifications including acetylation, therapies targeting this process (e.g., HDAC and bromodomain inhibitors) may be applicable to heart failure, where conduction defects most commonly leads to death.

We have recently reported on how proteotoxicity secondary to misfolded proteins, implicated in the pathologensis of multiple neurodegenerative diseases, mediates heart failure and may represent one such targetable intervention. In the present study, we identify the switching defective/sucrose non-fermenting (SWI/SNF) chromatin-remodeling apparatus of the adult cardiomyocyte as another potential target to consider.

Although SWI/SNF complexes exhibit considerable subunit heterogeneity, BRG1 (also known as SMARCA4) and BRM (also known as SMARCA2) are the only catalytic subunits with ATPase activity. SWI/SNF complexes are recruited by pioneer transcription factors to the enhancers and promoters of target genes, where they reposition nucleosomes in an ATP-dependent manner to positively or negatively regulate transcription. In the heart, SWI/SNF complexes physically interact with the transcription factors N10(2.5, TBX5, and GATA4, which make them a plausible candidate for regulating cardiomyocyte development in the embryo. The critical role of SWI/SNF complexes during development is evident in the embryonic lethality seen in constitutive mutations of SWI/SNF components, including Brg1. Since mutations in BRG1 and BRMresult in the syndromic congenital heart defects Coffin-Siris Syndrome and Nicolaides-Baraitser Syndrome, their role in supporting critical developmental transcription factors is clear. However, the role of BRG1 and BRM in the adult cardiomyocyte under basal conditions is currently unknown.

BRG1 and BRM SWI/SNF complexes have tissue-specific activities that depend upon their physiological context. Deletion of Brg1/Brm in endothelial cells attenuates the expression of cell adhesion molecules and adhesion of leukocytes, with BRG1/BRM supported NF-kB implicated in chromatin immunoprecipitation assays. In the present study, we provide the first studies implicating the mechanistic role of BRG1 and BRM in differentiated adult cardiomyoyctes using an inducible Brg1/Brm double-mutant mouse line specific for the cardiomyocyte (alpha MHC). In striking contrast to the effects of BRG1 and BRM deletion in vascular endothelial cells (generalized induction of apoptosis), here we identify specific conduction defects linked to the transcriptional regulation of c-Myc. We identify that BRG1/BRM has a role inhibiting c-Myc, as the absence of Brg1 and Brm led to a >1000-fold increase in expression. We then elucidated for the first time the role of c-Myc in inhibiting specific gap junction proteins and sodium channel expression involved in electrical conduction and metabolic cell-to-cell signaling. With evidence that BRG1/BRM occupancy is diminished at these conduction genes in an array of human heart failure cases, paralleling our animal model studies, we identify for the first time a novel epigenetic mechanism that maintains the coordinated transmission of the cardiomyocyte pacemaker in the heart.

BRG1- and BRM-catalyzed SWI/SNF complexes are required in cardiomyocytes for organismal survival. To investigate the combined role of BRG1 and BRM in adult cardiomyocytes, we generated Brg1^{fl/fl} mice carrying an inducible, cardiomyocyte-specific αMHC-Cre-ERT transgene that were also Brm^{-/-}. Conditional loss of Brg1 after 7 days of tamoxifen treatment was confirmed by PCR and IHC. These mice (herein referred to as Brg1/Brm double mutants), which are null for BRG1 and BRM in cardiomyocytes, died at 6-22 days with a mean of 11.6±1.5 days. Six groups of controls were analyzed in parallel including Brg1 and Brm single mutants, and each control group exhibited 100% survival. The rapid mortality of Brg1/Brm double mutants supports our initial hypothesis that BRG1 and BRM functionally compensate and that SWI/SNF complexes are essential for cardiomyocyte homeostasis.

BRG1 and BRM are required for electrophysiological homeostasis. To determine the precise role of SWI/SNF in cardiomyocytes, we monitored 27 Brg1/Brm double mutants by echocardiography on a daily basis. Every Brg1/Brm double-mutant mouse experienced a rapid and progressive decline in cardiac function that preceded early-onset death. Double mutants developed severe left ventricular (LV) systolic dysfunction as evidenced by decreased ejection fraction (EF) % and decreased fractional shortening % as well as LV dilation as evidenced by a widening LV that contracted less (Table 11). Interestingly, a characteristic bradycardia was identified in the 24 hours before the mice died (herein referred to as 1-day pre-mortem) (Table 12), which could be characterized by two cardiac phenotypes: 1) a dilated cardiomyopathy with severe dysfunction and significantly thinner walls (EF %<50%, mean 26.4±3.1%); and 2) a hypertrophic cardiomyopathy with less severe systolic dysfunction (EF %>50%, mean 69.1%) (Table 12). Both phenotypes had significantly decreased heart rates (422±28 and 532±40, respectively) by conscious echocardiography.

Six groups of control mice were analyzed by echocardiography in parallel. The only abnormality that was observed among controls was a mild transient effect in Group 5 associated with tamoxifen treatment of αMHC-Cre-ERT transgenic mice in the absence of a conditional floxed allele that has been previously described. However, it did not result in any mortality and baseline function was restored immediately upon tamoxifen withdrawal. These electrophysiologic data suggest Brg1/Brm double mutants have a defect in the conduction system, either at the level of the cardiomyocyte ion conduction channels or by the specialized cardiomyocyte-derived conduction system in the heart.

A link between BRG1/BRM and c-MYC. Considering the severity of the cardiac conduction phenotype and the rapid demise of Brg1/Brm double-mutant mice, the histopathology was surprisingly mild with no signs of cell death or structural damage to the tissues. Double-mutant hearts appeared normal with no significant changes in cellular composition or fibrosis. This is an important observation because it suggests that any alterations in cardiac gene expression in double mutants are not simply a secondary consequence of morphological defects.

To further delineate the mechanisms underlying the pathogenesis of the heart disease caused by ablating Brg1 and Brm in cardiomyocytes, we performed RNA-seq transcriptome analysis on hearts from Brg1/Brm double-mutant mice at an early time point (day 9) compared to multiple control groups. At this early time point, only 17 mRNAs were significantly different in the Brg1/Brm double-mutant hearts. An early time point was chosen to differentiate the direct role of BRG1- and BRM-catalyzed SWI/SNF complexes in the disease process from an indirect, secondary response the cardiomyocytes might have had due to these primary events. The use of multiple control groups further increased the rigor of this study, which contributed to the relatively small number of significant genes. Unexpectedly, TRANSFAC analysis of these 17 genes identified c-MYC binding sites in 14 of the 15 named genes (p=0.0007, Bayes Factor 5). This finding was interesting given that SWI/SNF has been recently shown to play a role in acute leukemia maintenance and enhancer-mediated c-Myc regulation, where BRG1 was found to be required to maintain transcription factor occupancy for long-range chromatin looping interactions with the c-Myc promoter.

To independently evaluate the functional importance of c-MYC, which is associated with cardiac hypertrophy, we analyzed a transgenic mouse model that inducibly overexpresses human c-MYC in adult cardiomyocytes. We confirmed that c-MYC expression was induced by DOX within 24 hours of treatment. This c-MYC induction significantly decreased the expression of the cardiac connexin Cx43 at the mRNA and protein levels. Importantly, MYC-ON transgenic mice exhibited rapid cardiac dysfunction compatible with CX43 downregulation. Continuous ECG telemetry revealed a significantly decreased heart rate and significant perturbations in PR interval, QRS duration, and QTc that culminated in a complete heart block by day 6. These findings indicate that c-MYC regulation is crucial for cardiac conduction and suggest a functional link between BRG1/BRM and c-MYC regulation in cardiomyocytes.

BRG1 and BRM regulate electrical conduction in cardiomyocytes. The next objective was to confirm whether SWI/SNF regulates cardiac conduction and to determine the cause of death of Brg1/Brm double mutants. Therefore, continuous ECG telemetry was performed and revealed significant repolarization abnormalities in Brg1/Brm double-mutants by 13 days after the loss of Brg1. For example, one Brg1/Brm double-mutant had a QT interval that was clearly >50% of the R-R interval at day 13, which is consistent with marked QT prolongation. Abnormal ST segment morphology, characterized by downsloping ST depression and T-wave inversion, was also evident. This time point also was characterized by relative bradycardia and mild widening of the QRS complex. These abnormalities persisted until day 17 at which time the mouse developed apparent sinus arrest with a slow ventricular escape rhythm. This terminal rhythm progressed to complete cessation of electrical activity within hours. Another Brg1/Brm double mutant demonstrated abnormalities in both atrioventricular (AV) conduction and ventricular repolarization by ECG analysis at day 13. The AV conduction abnormalities initially manifested as prolongation of the PR interval (1^{st} degree AV block) and progressed rapidly to complete heart block (3^{rd} degree AV block) with a slow junctional escape. This terminal rhythm progressed to complete cessation of electrical activity within minutes on day 13. This time point was characterized by repolarization abnormalities, initially manifest as markedly peaked (“hyperacute”) T waves, followed by ST segment depression and T wave inversion. The QRS interval also became mildly prolonged. It is important to note that the Brg1/Brm double-mutant ECG profiles phenocopy the MYC-ON profiles, which lends credence to a functional link between BRG1/BRM and c-MYC.

As an internal control, the Brg1/Brm double-mutant mice had baseline ECG measurements prior to loss of Brg1 that were indistinguishable from controls, which had a normal heart rate, PR interval, QRS duration, and corrected QT intervals at all of the time points collected. These ECG data demonstrate that BRG1- and BRM-catalyzed SWI/SNF complexes are required for cardiomyocyte conduction and provide a unique opportunity to understand how epigenetic mechanisms regulate this crucial process.

BRG1 and BRM directly and indirectly regulate cardiac conduction genes. To test the hypothesis that SWI/SNF complexes participate in a mechanism that regulates cardiac conduction genes, we analyzed c-Myc, Cx40, Cx43, and Scn5a expression levels. Indeed, Brg1/Brm double-mutant hearts had significantly increased c-Myc mRNA levels and significantly decreased Cx40, Cx43, and Scn5a mRNA levels 15 days after the loss of Brg1. We also tested whether Tbx5 or Nkx2. 5 expression were altered in adult hearts from Brg1/Brm double mutants. Indeed, Tbx5 mRNA levels were significantly lower in double mutants than controls.

During cardiomyocyte cell fate determination and differentiation, cardiogenic transcription factors recruit SWI/SNF complexes to a number of target genes. To determine whether this is the case for electrical conduction in the adult heart, we performed quantitative ChIP assays and observed BRG1/BRM occupancy at the promoters of the Cx40, Cx43, and Scn5a genes. These results indicate that BRG1 maintains the conduction system directly by binding to the promoters of these cardiac connexin and ion channel targets as well as indirectly by activating Tbx5 expression and repressing c-Myc expression.

Diminished BRG1/BRM occupancy and expression of cardiac conduction genes in human heart failure cases. To provide evidence that the BRG1/BRM regulatory mechanism is clinically relevant, we analyzed cardiac tissue from 10 human heart failure cases and 5 controls. The cases were selected on the basis of diagnosed bundle branch blocks and arrhythmias. The controls were obtained from heart transplant donors whose hearts could not be used due to the unavailability of a suitable recipient within the required timeframe. BRG1 and BRM expression levels were not significantly different between the cases and controls. However, c-MYC expression levels were increased in cases, while CX43 and SCN5A expression levels were decreased. The difference in c-MYC approached statistical significance despite the heterogeneity of the human clinical samples. Furthermore, when the cases were stratified into two groups, a subset corresponding to >50% of the cases had significantly higher c-MYC expression levels compared to controls. CX43 and SCN5A expression levels were significantly lower in all of the cases without stratification. However, these differences were more highly significant when a subset of >50% of the cases were compared to controls.

The existence of long noncoding RNAs (IncRNAs) that influence the occupancy of epigenetic enzymes at genomic loci raised the possibility that BRG1/BRM occupancy of cardiac conduction genes might be diminished in heart failure cases even if global BRG1/BRM expression levels were not significantly affected. To test this hypothesis, we performed quantitative ChIP assays on cardiac tissue from the same human clinical samples. BRG1/BRM occupied the promoter of the CX43 and SCN5A genes, and occupancy was significantly diminished in heart failure cases associated with bundle branch blocks and arrhythmias compared to controls. These results suggest that BRG1 and BRM directly and indirectly regulate the expression of cardiac conduction genes in humans and that perturbation of this mechanism contributes to heart failure and sudden death.

The mechanisms elucidated in the current study are summarized in the following working model: The BRG1 and BRM catalytic subunits of SWI/SNF complexes directly and indirectly activate the expression of Cx40, Cx43, and Scn5a to facilitate conduction in cardiomyocytes. The direct regulation is based on ChIP assays demonstrating BRG1 occupancy at each promoter. The indirect regulation is mediated by activation of Tbx5 (an activator) and inhibition of c-Myc (an inhibitor). These mechanisms highlight potential issues with inhibiting BRG1/BRM therapeutically, by linking chromatin remodeling with c-MYC regulation and cardiac arrhythmogenesis in vivo.

The inducible Brg1/Brm double-mutant mice reported here have been found to have increased linoleic acid and oleic acid and decreased glucose-6-phosphate, fructose-6-phosphate, and myoinositol by non-targeted metabolomics analysis. As these studies were performed at an early time point after tamoxifen-induced Brg1 deletion before the onset of acute disease, these studies suggest an increase in long-chain fatty acids and a reduction in cardiac glucose/glycogen stores. These Brg1/Brm metabolic changes may reflect their regulation of c-MYC. Our current work shows that c-MYC-overexpressing cardiomyocytes downregulate CX43 expression, resulting in lethal arrhythmias Our findings that BRG1 and BRM inhibit c-Myc expression and antagonize c-MYC in terms of Cx43 expression is reminiscent of their opposing functions in cancer where BRG1 and BRM function as tumor suppressors and c-MYC functions as a proto-oncogene. Since c-MYC is one of 5 hub nodes in transcriptional networks having a close relationship with heart failure, it may significantly contribute to the cardiac defects observed in Brg1/Brm double-mutant mice and human clinical samples in the current study.

Here we demonstrate for the first time that c-MYC regulates gap junction connexins and specific sodium channels involved in conduction linked to an arrhythmia phenotype in vivo.

Mice. The αMHC-Cre-ERT mice [also known as B6.Cg-Tg(Myh6-cre/Esr1)1JmkJ or αMHC-MerCreMer] were obtained from The Jackson Laboratory (#005657, Bar Harbor, Me.) and genotyped as previously described. The Brg1 conditional mutant mouse line and Brm constitutive mutant mouse line have been described previously. Genotyping of the Brg1 floxed and Δfloxed alleles and the Brm mutation were genotyped by PCR. To induce the Brg1 conditional mutation in adult cardiomyocytes, 3-6 month old male and female mice were provided rodent chow containing tamoxifen (Sigma-Aldrich #T5648, St. Louis, Mo.) over a 7-day period. The route of delivery and dose were selected to minimize a previously described artifact caused by high doses of tamoxifen in the presence of the αMHC-Cre-ERT transgene. Briefly, 500 mg of tamoxifen was mixed with 1 kg of ground-up rodent chow and then mixed with water, kneaded into pellets, and dried in a hood. Provided to mice ad libitum, the dose was estimated to be 80 mg/kg/day. After the 7-day treatment period, the tamoxifen-fortified chow was removed and replaced with the same chow lacking tamoxifen.

The bi-transgenic mouse line that inducibly overexpresses the human c-MYC cDNA in cardiomyocytes under the control of the αMHC promoter has been previously described. Mice were raised in the absence of doxycycline (Dox) to prevent developmental consequences from c-MYC overexpression. c-MYC was induced by feeding mice Dox-containing rodent chow (200 mg/kg, Bio-Serve, Frenchtown, N.J.) ad libitum. Dox had no effect on single transgenic littermates, which were used as controls in analyses performed in this study.

Echocardiography. Conscious cardiac transthoracic echocardiography was performed on mice at the indicated time points using a VisualSonics Vevo 2100 ultrasound biomicroscopy system (VisualSonics, Inc., Toronto, Ontario, Canada). Two-dimensional M-mode echocardiography was performed in the parasternal long-axis view at the level of the papillary muscle on loosely restrained mice. Anterior and posterior wall thickness was measured as distance from epicardial to endocardial leading edges. Left ventricular internal diameters were also measured. Left ventricular systolic function was assessed by ejection fraction (LV EF %=[(LV Vol; d-LV Vol; s/LV Vol; d)×100] and fractional shortening (% FS=[(LVEDD−LVESD)/LVEDD]×100). Investigators were blinded to mouse genotype from collection through waveform measurements. Each measurement represents the average of three cardiac cycles from each mouse.

Electrocardiography. Continuous electrocardiographies (ECGs) were monitored by surgically implanting a TA10ETA radiotelemetry device (Data Sciences International (DSI), St. Paul, Minn.) into the abdomen of mice anesthetized with isoflurane and transmitting the information to APR-1 receivers under the cages that were coupled to the Ponemah v.5.0 Physiology Platform for data analysis (DSI).

Histology. Histology was performed by fixing adult heart tissues in 4% paraformaldehyde or 10% formalin, embedding in paraffin, and cutting 5-μm sections according to standard procedures. Sections were either stained with hematoxylin and eosin (H&E), Mason's Trichrome, or processed for immunohistochemistry (IHC) using a BRG1 rabbit polyclonal antibody (Millipore #07-478, Temecula, Calif.).

RNA isolation. Cardiac tissues were homogenized using a TissueLyser LT (Qiagen N.V. #69980, Venlo, The Netherlands) according to the manufacturer's protocols. Approximately 20-40 mg of apical ventricle was homogenized in 1 mL of Trizol (Life Technologies #15596-026, Carlsbad, Calif.) using a 5-mm stainless steel bead (Qiagen N.V. #69989). Chloroform (200 μL) was added, centrifuged at 12,000 g (15 min at 4° C.), isopropanol (0.5 mL) was then added to the aqueous phase, centrifuged at 12,000 g (10 min at 4° C.) and the resulting RNA pellet was washed with 1 mL of 75% ethanol, centrifuged at 7500 g (5 min at 4° C.). The resulting pellet was dried and resuspended in RNase-free water. RNA concentrations were then determined by UV spectroscopy (absorbance of 260-280 nm).

RT-qPCR. RNAs (500 ng) were reverse-transcribed using iScript reverse transcription supermix (Bio-Rad Laboratories #170-8841, Hercules, Calif.). TaqMan gene expression assays (Life Technologies) were performed using universal TaqMan master mix (Life Technologies #4304437) Primer pairs and probes for RT-qPCR assays are listed in Supplemental Material.

RNA-seq transcriptomics. Total RNAs were extracted (Norgen #25700, Thorold, Ontario, Canada), and their integrities were confirmed using a BioAnalyzer (Agilent, Santa Clara, Calif.). A PolyA+ isolation kit (Promega #Z5300, Madison, Wis.) was used to enrich for mRNAs, and strand-specific RNA-seq libraries were prepared using a kit (Clontech #634836, Mountain View, Calif.). A QC step was performed using a Quibit 2.0 fluorometer (Life Technologies). Libraries were pooled at 2-nm concentrations, and the samples were then subjected to cBot cluster generation using TruSeq Rapid PE Cluster Kit (Illumina, San Diego, Calif.). The amplified libraries were sequenced at the High-Throughput Sequencing Facility at UNC using a HiSeq2500 instrument (Illumina) 50M paired-end reads were analyzed. RNA-seq data were aligned with MapSplice, and genes were quantified with RSEM. Gene expression estimates were upper quartile normalized.

Differential expression was assessed between samples by calculating a t-statistic for each gene. P-values from the t-tests were transformed to q-values to control for multiple hypothesis testing and evaluate the false discovery rate (FDR) of the resulting candidates. A q-value threshold of 0.05 was applied to identify differentially expressed genes. Differential expression was assessed between samples by SAM using the two-class unpaired option. A FDR threshold of 0.05 was applied to identify differentially expressed genes. For heat maps, cluster analysis of candidate genes was performed using average linkage hierarchical clustering and Pearson correlation.

ChIP Assays. ChIP assays were performed as previously described. Briefly, 10-30 mg of cardiac tissues were pulverized in liquid nitrogen using a mortar and pestle and then crosslinked in 1% formaldehyde at room temperature for 10 minutes. After the crosslinking reaction was stopped with 0.125M glycine, the tissues were lysed, and the chromatin was sonicated into 200-500-bp fragments. 5% of the sonicated chromatin was removed as input, and the remainder of each sample was immunoprecipitated overnight at 4° C. using a BRG1 antibody (J1), which cross-reacts with BRM. Duplicate samples were immunoprecipitated with rabbit IgG as a negative control Immunoprecipitants were pulled down using protein A/G beads (Santa Cruz Biotechnology), washed following standard procedures, and eluted in 10-25 μL of ddH₂O.

qPCR was performed using Power SYBR Green Master Mix (Life Technologies) using the following primer pairs. Mouse: Cx40: Forward, CTTTCTCGACTGGTGAGGAA (SEQ ID NO:9); Reverse, GAGCCTGTTAGTTGCTCCCG (SEQ ID NO:10) (450 nM final concentration of each). Cx43: Forward, CCCTTCTCGTCAGCACATTG (SEQ ID NO:11); Reverse, AGCCACTGACTCAACTGGAA (SEQ ID NO:12) (300 nM final concentration of each). Scn5a: Forward, GTCAGAGTGGTGGGCTG (SEQ ID NO:13); Reverse, GATCCCCACATCCCACGG (SEQ ID NO:14) (250 nM final concentration of each). Dissociation curves and agarose gels demonstrated a single PCR product in each case without primer dimers. Relative enrichment was determined by comparison to serial dilutions of input samples.

Statistics. SigmaPlot (Systat Software, Inc., San Jose, Calif.) was used to determine significant statistical difference by One-way ANOVA followed by post-hoc analysis using the Holm-Sidak method or a Student's t-test. A p value<0.05 was considered significant.

TagMan assay information. The method for RNA isolation and real-time RT-PCR was described in Kim et al., PNAS 99:4602-4607 (2002).
F: 5′-Fluorescein (FAM)
Q: Quencher (TAMRA)

### Mouse:

cMyc
(SEQ ID NO: 102)
Forward: CCA GCC CTG AGC CCC TAG T
(SEQ ID NO: 103)
Reverse: TGC TCT TCT TCA GAG TCG CT
(SEQ ID NO: 104)
Probe: FTG CAT GAG GAG ACA CCG CCC ACC AQ
Tbox5
(SEQ ID NO: 105)
Forward: CCA CTG TAC CAA GAG GAA AG
(SEQ ID NO: 106)
Reverse: TGT CTC CAT GTA CGG CTT CT
(SEQ ID NO: 107)
Probe: FAA TGT TCC AGC ACG GAG CAC CCC TAQ
Cx40
(SEQ ID NO: 108)
Forward: ACC ATC ATG GGC ATG ATC TG
(SEQ ID NO: 109)
Reverse: ATA GGT GAC CCT GCC AAG AC
(SEQ ID NO: 110)
Probe: FTG ATC GTG GAG GTC TTG CTG AGG ATG Q
Cx43
(SEQ ID NO: 111)
Forward: CCT CTT CAA GTC TGT CTT CGA
(SEQ ID NO: 112)
Reverse: TAG ACC GCA CTC AGG CTG AA
(SEQ ID NO: 113)
Probe: FTG GCC TTC CTG CTG ATC CAG TGG TAQ
Trpm 7
(SEQ ID NO: 114)
Forward: ATT CCC TTC GTT CCT GTA CC
(SEQ ID NO: 115)
Reverse: ACT GGG AGA ACT CTC CTC CA
(SEQ ID NO: 116)
Probe: FAC GAG GCG AGC CTG TCA CAG TGT ACQ
Sen5a
(SEQ ID NO: 117)
Common Forward: TCA CCA ACA GCT GGA ACA TC
(SEQ ID NO: 118)
Full Reverse: GGA GAF GAC AGT GCC AAC G
(SEQ ID NO: 119)
D Variant Reverse: AGA GCA ACG TGC GAA CAA CG
(SEQ ID NO: 120)
C Variant Reverse: CAA CAC CTG ACA TGT ACG CAT
(SEQ ID NO: 121)
Common Probe: FCG ATT TCG TGG TTG TCA TCC TCT CCQ
Gapdh
(SEQ ID NO: 57)
Forward: AGG TCG GTG TGA CCG GAT TT
(SEQ ID NO: 58)
Reverse: GGC AAC AAT CTC CAC TTT GC
(SEQ ID NO: 59)
Probe: FTG CAA ATG GCA GCC CTG GTG ACC AQ
Human:
BRG1
(SEQ ID NO: 122)
Forward: CGA AAG GAG CTG CCC GAG T
(SEQ ID NO: 123)
Reverse: TGG TTG CGA ATG CGC TCC T
(SEQ ID NO: 124)
Probe: FAC GAG CTC ATC CGC AAG CCC GTG Q
BRM
(SEQ ID NO: 125)
Forward: GCC GTG ACG TGG ACT ACA GT
(SEQ ID NO: 126)
Reverse: AAA TTG CCG TCT TCG ATG GC
(SEQ ID NO: 127)
Probe: FAC GCC CTC AGG GAG AAG CAG TGG Q
cMYC
(SEQ ID NO: 128)
Forward: TTC GGG TAG TGG AAA ACC AG
(SEQ ID NO: 129)
Reverse: GGT CAT AGT TCC TGT TGG TG
(SEQ ID NO: 130)
Probe: FTC CCG CGA CGA TGC CCC TCA ACG Q
SCN5A
(SEQ ID NO: 131)
Forward: CCA ACA GCT GGA ATA TCT TCG A
(SEQ ID NO: 132)
Reverse: TTC TGG ATG ATG TCC GAG AG
(SEQ ID NO: 133)
Probe: FTC GTG GTT GTC ATC CTC TCC ATC GTG Q
CX40
(SEQ ID NO: 134)
Forward: TCT TTA TGC TGG CTG TGG CT
(SEQ ID NO: 135)
Reverse: GAT CTT CTT CCA GCC CAG GT
(SEQ ID NO: 136)
Probe: FAC TGT CCC TCC TCC TTA GCC TGG CQ
CX43
(SEQ ID NO: 137)
Forward: AAG CAA AAG AGT GGT GCC CA
(SEQ ID NO: 138)
Reverse: CAG CAG TTG AGT AGG CTT GA
(SEQ ID NO: 139)
Probe: FTG TCA AGG AGT TTG CCT AAG GCG CTC Q
GAPDH
(SEQ ID NO: 99)
Forward: ACC TCA ACT ACA TGG TTT AC
(SEQ ID NO: 100)
Reverse: GAA GAT GGT GAT GGG ATT TC
(SEQ ID NO: 101)
Probe: FCA AGC TTC CCG TTC TCA GCC Q

TABLE 1
Descriptive statistics of reference gene expression by BestKeeper

| | Gapdh | 18S | Pgk1 |
|---|---|---|---|
| n | 24 | 24 | 24 |
| Geo mean | 20.03 | 15.65 | 24.91 |
| AR mean | 20.04 | 15.65 | 24.92 |
| Min (Cp) | 18.46 | 15.19 | 23.30 |
| Max (Cp) | 20.77 | 16.12 | 25.93 |
| SD (±Cp) | 0.40 | 0.24 | 0.52 |
| CV (% Cp) | 1.99 | 1.55 | 2.09 |
| Coeff. Of Corr. (r) | 0.941 | 0.791 | 0.980 |
| p value | 0.001 | 0.001 | 0.001 |

TABLE 2
High-resolution transthoracic echocardiography performed on conscious mice in Groups
1-5 at baseline and after initiating tamoxifen/chow or chow diet for 7 days.

| | Group 1 | Group 2 | Group 3 | | Group 4 | | Group 4 |
|---|---|---|---|---|---|---|---|
| | Brm−/− | Brm−/− | Brm−/− | Group 5 | Brm−/− | | Brm−/− |
| | flx/flx | flx/flx | flx/flx | Brm−/− | flx/flx | Groups 1-3, 5 | flx/flx |
| | No Tg | No Tg | Brg1 Tg+ | flx/+ | Brg1 Tg+ | (controls) | Brg1 Tg+ +TAM |
| | Chow Diet | +TAM | Chow | Brg1 Tg+ | +TAM | Day Matched | 1 Day Pre-Mortem |
| | Baseline | Baseline | Baseline | Baseline | Baseline | Pre-Mortem | (Day 11.6 ± 1.5) |
| | N = 3 | N = 14 | N = 5 | N = 7 | N = 30 | N = 28 | N = 27 |
| AWTS (mm) | 1.73 ± 0.10 | 1.77 ± 0.04 | 1.92 ± 0.13 | 1.62 ± 0.04 | 1.84 ± 0.04 | 1.83 ± 0.04 | 1.37 ± 0.10^{§} |
| LVESD (mm) | 1.28 ± 0.04 | 1.28 ± 0.04 | 1.24 ± 0.01 | 1.16 ± 0.08 | 1.29 ± 0.03 | 1.22 ± 0.03 | 3.16 ± 0.24^{§} |
| PWTD (mm) | 1.12 ± 0.03 | 1.09 ± 0.02 | 1.18 ± 0.07 | 0.97 ± 0.05 | 1.06 ± 0.02 | 1.10 ± 0.02 | 1.01 ± 0.06 |
| PWTS (mm) | 1.91 ± 0.06 | 1.78 ± 0.02 | 1.78 ± 0.06 | 1.60 ± 0.06 | 1.68 ± 0.03 | 1.77 ± 0.03 | 1.27 ± 0.07^{§} |
| LV Mass (mg) | 110.9 ± 4.9 | 114.1 ± 3.7 | 126.9 ± 11.1 | 87.9 ± 7.1 | 117.8 ± 5.3 | 111.9 ± 4.0 | 156.0 ± 9.1^{§} |
| LV Mass/BW | 3.1 ± 0.2 | 3.9 ± 0.1 | 4.1 ± 0.4 | 3.9 ± 0.2 | 4.2 ± 0.1 | 3.9 ± 0.1 | 6.2 ± 0.3^{§} |
| (mg/g) | | | | | | | |
| BW | 36.1 ± 0.6 | 29.7 ± 1.2 | 31.2 ± 0.7 | 22.5 ± 0.9 | 27.7 ± 1.0 | 28.8 ± 0.9 | 25.4 ± 0.9 |

Data represent means ± SEM.
A One Way Analysis of Variance was performed, followed by an all pairwise multiple comparison procedure (Holm-Sidak method), when overall significance <0.05.
^{§}p < 0.001 vs. all other groups.
HR, heart rate; ExLVD, external left ventricular diameter; bpm, heart beats per minute; AWTD, anterior wall thickness in diastole; AWTS, anterior wall thickness in systole; PWTD, posterior wall thickness in diastole; PWTS, posterior wall thickness in systole; LVEDD, left ventricular end-diastolic dimension; LVESD, left ventricular end-systolic dimension; FS, fractional shortening, calculated as (LVEDD − LVESD)/LVEDD × 100; EF %, ejection fraction calculated as (end Simpson's diastolic volume − end Simpson's systolic volume)/end Simpson's diastolic volume * 100, ND, not determined.

TABLE 3
Two phenotypic subgroups of Brm/Brg1 double mutant mice at 1-day pre-
mortem. High-resolution transthoracic echocardiography performed on
conscious mice 1 day pre-mortem (on time matched controls) after initiating
tamoxifen/chow or chow diet for 7 days. Data represent means ± SEM.
A One Way Analysis of Variance was performed, followed by an all pairwise
multiple comparison procedure (Holm-Sidak method), when overall significance <0.05.

| | | | 2 | | |
|---|---|---|---|---|---|
| | | | Phenotypic | | |
| | | | Subsets | | |
| | | Group 4 | Group 4 | Subset 1. | Subset 2. |
| | | Brm−/− flx/flx | Brm/Brg1 | Group 4 | Group 4 |
| | | Brg1 Tg+ | Double | Brm/Brg1 | Brm/Brg1 |
| | | +TAM | Mutants | Double | Double |
| | Groups 1-3, 5 | 1 Day Pre- | 1. <50% | Mutants | Mutants |
| | (CONTROLS) | Mortem | EF | EF % <50% | EF >50% |
| | Day Matched | (Day | 2. >50% | 1 Day Pre- | 1 Day Pre- |
| | Pre-Mortem | 11.6 ± 1.5) | EF | Mortem | Mortem |
| | N = 28 | N = 27 | → | N = 17 | N = 10 |
| AWTS | 1.83 ± 0.04§ | 1.37 ± 0.10 | → | 1.14 ± 0.11** | 1.87 ± 0.13§ |
| (mm) | | | | | |
| LVESD | 1.22 ± 0.03 | 3.16 ± 0.24**,† | → | 4.17 ± 0.37* | 1.84 ± 0.21 |
| (mm) | | | | | |
| PWTD | 1.10 ± 0.02 | 1.01 ± 0.06 | → | 0.93 ± 0.07† | 1.23 ± 0.11 |
| (mm) | | | | | |
| PWTS | 1.77 ± 0.03* | 1.27 ± 0.07§ | → | 1.17 ± 0.07† | 1.56 ± 0.10 |
| (mm) | | | | | |
| LV Mass | 111.9 ± 4.0* | 156.0 ± 9.1 | → | 150.2 ± 15.0 | 148.0 ± 13.6 |
| (mg) | | | | | |
| LV | 3.9 ± 0.1* | 6.2 ± 0.3 | → | 6.0 ± 0.3 | 6.4 ± 0.7 |
| Mass/BW | | | | | |
| (mg/mm) | | | | | |
| BW | 28.8 ± 0.9 | 25.4 ± 0.9 | → | 26.4 ± 1.1 | 23.7 ± 1.5** |

*p < 0.001 vs. all other groups;
**p < 0.05 vs. Column 1;
§p < 0.05 vs. Column 2;
†p < 0.01 vs. Column 4.
HR, heart rate;
ExLVD, external left ventricular diameter;
bpm, heart beats per minute;
AWTD, anterior wall thickness in diastole;
AWTS, anterior wall thickness in systole;
PWTD, posterior wall thickness in diastole;
PWTS, posterior wall thickness in systole;
LVEDD, left ventricular end-diastolic dimension;
LVESD, left ventricular end-systolic dimension;
FS, fractional shortening, calculated as (LVEDD − LVESD)/LVEDD × 100;
EF % , ejection fraction calculated as (end Simpson's diastolic volume − end Simpson's systolic volume)/end Simpson's diastolic volume * 100,
ND, not determined.

TABLE 4
High-resolution transthoracic echocardiography performed on conscious mice in
Groups 1-5 at baseline and after initiating tamoxifen/chow or chow diet for 7 days

| | | | | | GBrg1/Brmroup 3 |
|---|---|---|---|---|---|
| | | | | | DM |
| | | | Group 3 | Group 1, 2 | Brg1^{floxed/floxed}; |
| | Group 1 | Group 2 | Brg1^{floxed/floxed}; | (Controls) | αMHC-Cre- |
| | Brg1^{floxed/floxed}; | Brg1^{floxed/floxed}; | αMHC-Cre- | Day matched pre- | ERT+/0; Brg1/Brm^{-/-} |
| | αMHC-Cre- | αMHC-Cre- | ERT+/0; Brm^{-/-} | mortem | +TAM |
| | ERT+/0; Brm^{-/-} | ERT0/0; Brm^{-/-}) | +TAM | Group 1 N = 5 | 1 Day pre-mortem |
| | No TAM | +TAM | (Brg1/Brm DM) | Group 2 N = 10 | (day 11.6 ± 1.5) |
| | N = 3 | Baseline N = 5 | Baseline N = 5 | Total N = 15 | Total N = 10 |
| AWTD (mm) | 1.10 ± 0.02 | 1.06 ± 0.03 | 1.06 ± 0.03 | 1.11 ± 0.03 | 1.03 ± 0.08 |
| AWTS (mm) | 1.79 ± 0.06 | 1.82 ± 0.05 | 1.76 ± 0.11 | 1.87 ± 0.05 | 1.42 ± 0.15^{#} |
| LVEDD | 2.93 ± 0.17 | 3.13 ± 0.11 | 2.76 ± 0.13 | 2.83 ± 0.08 | 4.19 ± 0.22* |
| (mm) | | | | | |
| LVESD (mm) | 1.25 ± 0.01 | 1.31 ± 0.04 | 1.16 ± 0.07 | 1.21 ± 0.04 | 3.35 ± 0.30* |
| PWTD (mm) | 1.11 ± 0.01 | 1.07 ± 0.04 | 1.02 ± 0.02 | 1.12 ± 0.02 | 1.02 ± 0.08 |
| PWTS (mm) | 1.81 ± 0.07 | 1.82 ± 0.03 | 1.60 ± 0.05 | 1.81 ± 0.04 | 1.23 ± 0.10* |
| EF % | 88.6 ± 0.6 | 89.0 ± 0.2 | 88.4 ± 0.5 | 88.6 ± 0.5 | 42.0 ± 6.4* |
| FS % | 56.3 ± 1.0 | 58.0 ± 0.4 | 58.2 ± 0.7 | 57.3 ± 0.7 | 21.3 ± 3.6* |
| LV mass | 116.9 ± 7.0 | 120.6 ± 4.9 | 98.1 ± 9.5 | 113.0 ± 4.8 | 177.9 ± 15.4* |
| (mg) | | | | | |
| LV vol; d (μl) | 33.2 ± 2.8 | 34.1 ± 3.3 | 78.5 ± 7.6 | 30.8 ± 2.0 | 81.0 ± 9.2* |
| LV vol; s (μl) | 3.7 ± 0.1 | 4.3 ± 0.3 | 3.1 ± 0.5 | 3.6 ± 0.3 | 51.0 ± 9.4* |
| LV mass/BW | 3.7 ± 0.1 | 3.8 ± 0.2 | 3.9 ± 0.3 | 3.6 ± 0.1 | 6.9 ± 0.5* |
| (mg/g) | | | | | |
| BW | 31.5 ± 0.9 | 32.0 ± 0.8 | 25.6 ± 2.3 | 31.2 ± 1.0 | 26.1 ± 1.7 |
| HR (bpm) | 707 ± 45 | 707 ± 13 | 690 ± 9 | 744 ± 11 | 484 ± 40* |

Data represent mean ± SEM. A one way analysis of variance was performed, followed by an all pairwise multiple comparison procedure (Holm-Sidak method), when overall significance <0.05
HR heart rate,
ExLVD external left ventricular diameter,
bpm heart beats per minute,
AWTD anterior wall thickness in diastole,
AWTS anterior wall thickness in systole,
PWTD posterior wall thickness in diastole,
PWTS posterior wall thickness in systole,
LVEDD left ventricular end-diastolic dimension,
LVESD left ventricular end-systolic dimension,
FS fractional shortening calculated as (LVEDD − LVESD)/LVEDD × 100,
EF % ejection fraction calculated as (end Simpson's diastolic volume − end Simpson's systolic volume)/end Simpson's diastolic volume * 100,
ND not determined
*p < 0.001 versus all other groups;
^{#}versus Groups 1, 2 Controls day matched pre-mortem.

TABLE 5
VIP significant metabolites in Brm/Brg1 double mutant mice (Flx/Flx, Cre Tg+,
+tamoxifen) 12 days after feeding initiated (7 days total, Groups 1 and 3 only). Brm/Brg1
double mutant

| Control Group 1 (Flx/Flx, No Brg1 | Control Group 2 (Flx/Flx, No Brg1 | Control Groups 1 & 2 versus |
|---|---|---|
| Tg, Chow + tamoxifen) versus | Tg+, Chow diet - no tamoxifen) | Brm/Brg1 double mutant |
| Brm/Brg1 double mutant (VIP | versus Brm/Brg1 double mutant (VIP | (Chow + tamoxifen) |
| Rank) | Rank) | (VIP Rank) |
| Phosphoric Acid (1) | Phosphoric acid (1) | * |
| Alpha-monostearin (2) | * | Alpha-monostearin (5) |
| Urea (3) | Urea (11) | * |
| Glutamic acid (4) | Glutamic acid (2) | * |
| Lactic acid (5) | Lactic acid (7) | * |
| Cholesterol (6) | * | Cholesterol (12) |
| Stearic acid (7) | * | Stearic acid (15) |
| Creatinine (8) | Creatinine (5) | Creatinine (1) |
| Palmitic acid (9) | * | * |
| Linoleic acid (10) | Linoleic acid (15) | Linoleic acid (6) |
| Glucose-6-phosphate (11) | Glucose-6-phosphate (9) | Glucose-6-phosphate (4) |
| Fructose-6-phosphate (12) | * | Fructose-6-phosphate (9) |
| Alpha-monopalmitin (13) | * | Alpha-monopalmitin (18) |
| Oleic acid (14) | Oleic acid (16) | Oleic acid (11) |
| Serine (15) | Serine (14) | Serine (7) |
| * | 2-Aminoadipic acid (3) | 2-Aminoadipic acid (3) |
| * | Myoinositol (4) | Myoinositol (10) |
| * | Taurine (6) | Taurine (2) |
| * | Aldohexose (8) | Aldohexose (8) |
| * | Malic acid (10) | Malic acid (14) |
| * | Glycerol-1-phosphate (12) | * |
| * | Alanine (13) | Alanine (13) |
| * | * | Adenosine (16) |
| * | * | Threonine (17) |

The ranking (1-15) indicates the ranking given to the metabolites in their respective groups (Control Group 1, Control Group 2, Brg/Brm Double mutant Group 3)
Bold present in all three analyses
* Indicates this metabolite was not found to be a VIP significant metabolite

TABLE 6
VIP significant metabolites that were also t test significant (p < 0.05) in the three
comparisons made in this study, along with the related matched pathways from pathway
enrichment analysis

| Comparison | Metabolite (p value) | Pathway (hits) | p value |
|---|---|---|---|
| Control 1 versus Brg1/Brm | Oleic acid (0.005) | Biosynthesis of unsaturated fatty acids (2) | 0.0009 |
| double mutant | Linoleic acid (0.008) | Linoleic acid metabolism (1) | 0.0080 |
| | | Fatty acid biosynthesis (1) | 0.0598^{a} |
| Control 2 versus Brg1/Brm | Alanine (0.005) | Biosynthesis of unsaturated fatty acids (2) | 0.0049 |
| double mutant | Oleic acid (0.010) | Linoleic acid metabolism (1) | 0.0168 |
| | Linoleic acid (0.0014) | Ascorbate and aldarate metabolism (1) | 0.0252 |
| | Myoinositol (0.0021) | Galactose metabolism (1) | 0.0715^{a} |
| | | Inositol phosphate metabolism (1) | 0.0768^{a} |
| | | Fatty acid biosynthesis (1) | 0.1161^{a} |
| Controls 1 & 2 versus Brg1/Brm | Oleic acid (0.007) | Biosynthesis of unsaturated fatty acids (2) | 0.0081 |
| double mutant | Linoleic acid (0.010) | Linoleic acid metabolism (1) | 0.0210 |
| | Fructose-6-phosphate | Amino sugar and nucleotide sugar | 0.1241^{a} |
| | (0.016) | metabolism (1) | |
| | Creatinine (0.017) | Fatty acid biosynthesis (1) | 0.1429^{a} |
| | Alanine (0.033) | | |

VIP significance was based on the analysis of Control group 1 (Flx/Flx, No Brg1 Tg, Chow + tamoxifen, N = 3), Control Group 2 (Flx/Flx, Brg1 Tg+, Chow diet - no tamoxifen, N = 3), Control Groups 1 & 2 (Flx/Flx, No Brg1 Tg, Chow + tamoxifen AND Flx/Flx, Brg1 Tg+, Chow diet - no tamoxifen, N = 6), and Brg1/Brm Double Mutant (Chow + tamoxifen, N = 5) hearts
^{a}Not significant

TABLE 7
High-resolution transthoracic echocardiography performed on conscious mice in Groups
1-5 at baseline and after initiating tamoxifen/chow or chow diet for 7 days.

| | Group 1 | Group 2 | Group 3 | | Group 4 | | Group 4 |
|---|---|---|---|---|---|---|---|
| | Brm−/− | Brm−/− | Brm−/− | Group 5 | Brm−/− | | Brm −/− |
| | flx/flx | flx/flx | flx/flx | Brm−/− | flx/flx | Groups 1-3, 5 | flx/flx |
| | No Tg | No Tg | Brg1 Tg+ | flx/+ | Brg1 Tg+ | (CONTROLS) | Brg1 Tg+ +TAM |
| | Chow Diet | +TAM | Chow | Brg1 Tg+ | +TAM | Day Matched | 1 Day Pre-Mortem |
| | Baseline | Baseline | Baseline | Baseline | Baseline | Pre-Mortem | (Day 11.6 ± 1.5) |
| | N = 3 | N = 14 | N = 5 | N = 7 | N = 30 | N = 28 | N = 27 |
| AWTS (mm) | 1.73 ± 0.10 | 1.77 ± 0.04 | 1.92 ± 0.13 | 1.62 ± 0.04 | 1.84 ± 0.04 | 1.83 ± 0.04 | 1.37 ± 0.10^{§} |
| LVESD (mm) | 1.28 ± 0.04 | 1.28 ± 0.04 | 1.24 ± 0.01 | 1.16 ± 0.08 | 1.29 ± 0.03 | 1.22 ± 0.03 | 3.16 ± 0.24^{§} |
| PWTD (mm) | 1.12 ± 0.03 | 1.09 ± 0.02 | 1.18 ± 0.07 | 0.97 ± 0.05 | 1.06 ± 0.02 | 1.10 ± 0.02 | 1.01 ± 0.06 |
| PWTS (mm) | 1.91 ± 0.06 | 1.78 ± 0.02 | 1.78 ± 0.06 | 1.60 ± 0.06 | 1.68 ± 0.03 | 1.77 ± 0.03 | 1.27 ± 0.07^{§} |
| LV Mass (mg) | 110.9 ± 4.9 | 114.1 ± 3.7 | 126.9 ± 11.1 | 87.9 ± 7.1 | 117.8 ± 5.3 | 111.9 ± 4.0 | 156.0 ± 9.1^{§} |
| LV Mass/BW | 3.1 ± 0.2 | 3.9 ± 0.1 | 4.1 ± 0.4 | 3.9 ± 0.2 | 4.2 ± 0.1 | 3.9 ± 0.1 | 6.2 ± 0.3^{§} |
| (mg/g) | | | | | | | |
| BW | 36.1 ± 0.6 | 29.7 ± 1.2 | 31.2 ± 0.7 | 22.5 ± 0.9 | 27.7 ± 1.0 | 28.8 ± 0.9 | 25.4 ± 0.9 |

Data represent means ± S.E.M.
A one-way analysis of variance was performed, followed by an all pairwise multiple comparison procedure (Holm-Sidakmethod), when overall significance <0.05.
^{§}P < .001 vs. all other groups.
HR, heart rate; ExLVD, external left ventricular diameter; bpm, heart beats per minute; AWTD, anterior wall thickness in diastole; AWTS, anterior wall thickness in systole; PWTD, posterior wall thickness in diastole; PWTS, posterior wall thickness in systole; LVEDD, left ventricular end-diastolic dimension; LVESD, left ventricular end-systolic dimension; FS, fractional shortening, calculated as (LVEDD − LVESD)/LVEDD × 100; EF %, ejection fraction calculated as (end Simpson's diastolic volume − end Simpson's systolic volume)/end Simpson's diastolic volume × 100; ND, not determined.

TABLE 8
Descriptive statistics of reference gene expression by BestKeeper
and Primer Probe sets used on mouse heart samples.

| | Gapdh | 18S | Pgkl |
|---|---|---|---|
| n | 24 | 24 | 24 |
| Geo mean | 20.03 | 15.65 | 24.91 |
| AR mean | 20.04 | 15.65 | 24.92 |
| Min (Cp) | 18.46 | 15.19 | 23.30 |
| Max (Cp) | 20.77 | 16.12 | 25.93 |
| SD (±Cp) | 0.40 | 0.24 | 0.52 |
| CV (% Cp) | 1.99 | 1.55 | 2.09 |
| Coeff. Of Corr. (r) | 0.941 | 0.791 | 0.980 |
| P value | 0.001 | 0.001 | 0.001 |

qPCR Primer and Probe Sets (Mouse):

Atf6
(SEQ ID NO: 15)
Forward: TAC GTT GTC TCA TTT CGA AGG
(SEQ ID NO: 16)
Reverse: CAT TTT TGG TCT TGT GGT CTT G
(SEQ ID NO: 17)
Probe: FCA TCT GCT TAT TAC CAG CTA CCA CCC AQ
Atf3
(SEQ ID NO: 18)
Forward: GCC TGC CGA AAG AGT CAG AG
(SEQ ID NO: 19)
Reverse: TCT CAT TCT TCA GCT CCT CAA
(SEQ ID NO: 20)
Probe: FTG GGC CTT CAG CTC AGC ATT CAC ACQ
Ire1a
(SEQ ID NO: 21)
Forward: TCC GAG CCA TGA GAA ACA AG
(SEQ ID NO: 22)
Reverse: GAG CCC AGC GTC TCC TGA A
(SEQ ID NO: 23)
Probe: FAC CAC TAC CGG GAG CTC CCC GTG Q
Xbp-1 Full, Spliced
(SEQ ID NO: 24)
Common Forward: GTT CCA GAG GTG GAG GCC A
(SEQ ID NO: 25)
Full Reverse: TAG TCT GAG TGC TGC GGA CT
(SEQ ID NO: 26)
Spliced Reverse: GCC TGC ACC TGA CTC AGC AG
(SEQ ID NO: 27)
Common Probe: FAC CCG GCC ACC AGC CTT ACT CCA Q
Chop
(SEQ ID NO: 28)
Forward: CGA AGA GGA AGA ATC AAA AAC C
(SEQ ID NO: 29)
Reverse: TGT GAC CTC TGT TGG CCC T
(SEQ ID NO: 30)
Probe: FAC TAC TCT TGA CCC TGC GTC CCT AGQ
Atf4
(SEQ ID NO: 31)
Forward: CCT CGG AAT GGC CGG CTA T
(SEQ ID NO: 32)
Reverse: GGA AAA GGC ATC CTC CTT G
(SEQ ID NO: 33)
Probe: FAT GAT GGC TTG GCC AGT GCC TCA GQ
Grp78
(SEQ ID NO: 34)
Forward: ATA AAC CCC GAT GAG GCT GT
(SEQ ID NO: 35)
Reverse: ACC AGA TCA CCT GTA TCC TG
(SEQ ID NO: 36)
Probe: FCT ATG GTG CCG CTG TCC AGG CTG Q
PPARg1
(SEQ ID NO: 37)
Forward: CTG ACG GGT TCT CGG TTG A
(SEQ ID NO: 38)
Reverse: ATC AGT GGT TCA CCG CTT CT
(SEQ ID NO: 39)
Probe: FCT GAG AAG TCA CGT TCT GAC AGG ACQ
Fabp4
(SEQ ID NO: 40)
Forward: CAC CGA GAT TTC CTT CAA ACT
(SEQ ID NO: 41)
Reverse: GCC ATC TAG GGT TAT GAT GC
(SEQ ID NO: 42)
Probe: FCG TGG AAT TCG ATG AAA TCA CGC GCA Q
Pref-1
(SEQ ID NO: 43)
Forward: TCT GCG AAA TAG ACG TTC GG
(SEQ ID NO: 44)
Reverse: TTC GTA CTG GCC TTT CTC CA
(SEQ ID NO: 45)
Probe: FCT CAA CCC CCT GCG CCA ACA ATG Q
Cebpa
(SEQ ID NO: 46)
Forward: CAG CAA CGA GTA CCG GGT A
(SEQ ID NO: 47)
Reverse: TGC GTC TCC ACG TTG CGT T
(SEQ ID NO: 48)
Probe: FCA ACA ACA TCG CGG TGC GCA AGA GCC Q
Cebpb
(SEQ ID NO: 49)
Forward: CTG AGC GAC GAG TAC AAG AT
(SEQ ID NO: 50)
Reverse: GCG TCT CCA GGT TGC GCA
(SEQ ID NO: 48)
Probe: FCA ACA ACA TCG CGG TGC GCA AGA GCC Q
Collagen Type Ia
(SEQ ID NO: 51)
Forward: AGA GCA TGA CCG ATG GAT TC
(SEQ ID NO: 52)
Reverse: ATT AGG CGC AGG AAG GTC AG
(SEQ ID NO: 53)
Probe: FCT CCG ACC CCG CCG ATG TCG Q
18S
(SEQ ID NO: 54)
Forward: AGA AAC GGC TAC CAC ATC CA
(SEQ ID NO: 55)
Reverse: CTC GAA AGA GTC CTG TAT TGT
(SEQ ID NO: 56)
Probe: FAG GCA GCA GGC GCG CAA ATT ACQ
GAPDH
(SEQ ID NO: 57)
Forward: AGG TCG GTG TGA CCG GAT TT
(SEQ ID NO: 58)
Reverse: GGC AAC AAT CTC CAC TTT GC
(SEQ ID NO: 59)
Probe: FTG CAA ATG GCA GCC CTG GTG ACC AQ
β-Actin
(SEQ ID NO: 60)
Forward: CTG CCT GAC GGC CAG GTC
(SEQ ID NO: 61)
Reverse: CAA GAA GGA AGG CTG GAA AAG A
(SEQ ID NO: 62)
Probe: FCA CTA TTG GCA ACG AGC GGT TCC GQ
F: 5′-Fluorescein (FAM)
Q: Quencher (TAMRA)
* The method for Real-time RT-PCR was described
in Kim et al., PNAS 99:4602-4607 (2002).

TABLE 9
Primer Probe sets used on human heart samples.
qPCR Primer and Probe sets (human):

Atg5
Forward: CAA GAA GAC ATT AGT GAG ATA TGG (SEQ ID NO: 63)
Reverse: GCA AGA AGA TCA AAT AGC AAA CC (SEQ ID NO: 64)
Probe: FAT ATG AAG GCA CAC CAC TGA AAT GGC AQ (SEQ ID NO: 65)
Atg7
Forward: AGC AGC AGT GAC GAT CGG AT (SEQ ID NO: 66)
Reverse: CGT GAA AGA AAT CCC CGG AT (SEQ ID NO: 67)
Probe: FTG AGC CTC CAA CCT CTC TTG GGC Q (SEQ ID NO: 68)
Atg12
Forward: TAT GTG AAT CAG TCC TTT GCT C (SEQ ID NO: 69)
Reverse: CCA GTT TAC CAT CAC TGC CA (SEQ ID NO: 70)
Probe: FTC CCC AGA CCA AGA AGT TGG AAC TCQ (SEQ ID NO: 71)
Beclin1
Forward: GCA TAT GGC ATG ATA GCC TC (SEQ ID NO: 72)
Reverse: TCC ACA TGG CAT TAA TCT CCT (SEQ ID NO: 73)
Probe: FAT CAC CAG TAT CTT CAG CCC CAG GQ (SEQ ID NO: 74)
Vps34
Forward: GCA CTT GAA CCA GAT AAA ACT GT (SEQ ID NO: 75)
Reverse: GCA TGT AAT GCA CAG CCT CT (SEQ ID NO: 76)
Probe: FTC CGA CAG GTC TAA GCG GAA TTT ATC CQ (SEQ ID NO: 77)
Gabarapl1
Forward: GAC GCC TTA TTC TTC TTT GTC A (SEQ ID NO: 78)
Reverse: CTC ATG ATT GTC CTC ATA CAG (SEQ ID NO: 79)
Probe: FAC CAT CCC TCC CAC CAG TGC TAC CAQ (SEQ ID NO: 80)
Grp78
Forward: GCT TCT GAT AAT CAA CCA ACT G (SEQ ID NO: 81)
Reverse: TGT ACC CAG AAG ATG ATT GTC (SEQ ID NO: 82)
Probe: FAG GTC TAT GAA GGT GAA AGA CCC CTG Q (SEQ ID NO: 83)
Xbp-1 Spliced
Forward: GAA GAG GAG GCG GAA GCC AA (SEQ ID NO: 84)
Reverse: GCC TGC ACC TGA CTC AGC A (SEQ ID NO: 85)
Probe: FAC CCG GCC ACT GGC CTC ACT TCA Q (SEQ ID NO: 86)
Xbp-1 Full
Forward: GAA GAG GAG GCG GAA GCC AA (SEQ ID NO: 84)
Reverse: TAG TCT GAG TGC TGC GGA CT (SEQ ID NO: 25)
Probe: FAC CCG GCC ACT GGC CTC ACT TCA Q (SEQ ID NO: 86)
Cebpa
Forward: AGG CTC GCC ATG CCG GGA (SEQ ID NO: 87)
Reverse: GCT CCG CCT CGT AGA AGT (SEQ ID NO: 88)
Probe: FAC TCT AAC TCC CCC ATG GAG TCG GQ (SEQ ID NO: 89)
Cebpb
Forward: ATA AAT AAC CGG GCT CAG GAG (SEQ ID NO: 90)
Reverse: CCT CGG GTG GGT CCC CTT (SEQ ID NO: 91)
Probe: FTT AGC GAG TCA GAG CCG CGC ACG Q (SEQ ID NO: 92)
Chop
Forward: CTG GAA ATG AAG AGG AAG AAT C (SEQ ID NO: 93)
Reverse: TGG TTC TGG CTC CTC CTC A (SEQ ID NO: 94)
Probe: FTC ACC ACT CTT GAC CCT GCT TCT CTG Q (SEQ ID NO: 95)
AO
Forward: TGC CTG CAG AAA GAG TCG GA (SEQ ID NO: 96)
Reverse: CTT CTC GTT CTT GAG CTC CT (SEQ ID NO: 97)
Probe: FCT GAG CCT TCA GTT CAG CAT TCA CAC Q (SEQ ID NO: 98)
Gapdh
Forward: ACC TCA ACT ACA TGG TTT AC (SEQ ID NO: 99)
Reverse: GAA GAT GGT GAT GGG ATT TC (SEQ ID NO: 100)
Probe: FCA AGC TTC CCG TTC TCA GCC Q (SEQ ID NO: 101)
F: 5′-Fluorescein (FAM)
Q: Quencher (TAMRA).

TABLE 10
Summary of patient clinical characteristics and arrhythmia description heart
samples assayed in the present study came from.

| Group | Clinical Characteristics | Arrhythmia |
|---|---|---|
| Control 1 | Cerebral aneurysm; nonsmoker; not diabetic; heart normal size; BMI = 24.14 | None |
| Control 2 | EF = −55% ; heart normal size and no hypertrophy; nonsmoker; nondiabetic; | None |
| | BMI = 29.7 | |
| Control 3 | Non-failed- NF; HTN; ICH/stroke | None |
| Control 4 | Non-failed- NF; HTN; ICH/stroke | None |
| Control 5 | Non-failed- EF = 55% ; healthy tissue | None |
| Heart Failure | Failed- s/p CABG; Cardiomegaly w/mild assymmetric pulm. Edema | Prolonged QT; |
| Patient 1 | (L>R); DMII (NIDDM); HTN; CAD; nonsmoker | S and T wave |
| | | abnormality |
| Heart Failure | Failed- ICM; CAD; HTN; s/p ICD; s/p IABP; smoker; anterior STEMI w/ | Cardiogenic |
| Patient 2 | occlusion of prox LAD; COPD, HLD | shock and |
| | | bradycardia |
| Heart Failure | Failed- ISC Dilated cardiomyopathy; COPD; CAD; Pulm Embolism; s/p | Left bundle |
| Patient 3 | ICD; gout; DMII; GERD; previous MI; Vtach; s/p RVAD; Acquired Von | branch block; |
| | Willebrand; ex-smoker; EF 10% ; cardiac arrest | Ventricular |
| | | fibrillation |
| Heart Failure | Failed- ICM; Acute MI with cardiogenic shock (NSTEMI); NYHA stage | Right bundle |
| Patient 4 | IV; on VA ECMO; IDDM (DM type 1); acute renal failure; spinal stenosis; | branch block |
| | personal history of ECMO; nonsmoker | |
| Heart Failure | Failed- NICM; diabetes DM2 (NIDDM); HTN (controlled); CAD; | Non-sustained |
| Patient 5 | Cardiogenic shock; dyspnea; CKD; former smoker (quit 1994); gout; | Ventricular |
| | COPD; NYHA IV; s/p AICD; atrial fibrillation; EF <15% | Tachycardia |
| | | (NSVT) |
| Heart Failure | Failed- NICM; EF = 15-20% ; severe global enlargement; moderate MR and | Atrial |
| Patient 6 | TR; s/p IABP prior to surgery; s/p MVC; traumatic brain injury (TBI); | fibrillation |
| | elevated hemidiaphragm; HTN; hypercalcemia; obese; Gout; acute | (paroxysmal); |
| | decompensated heart failure (ADHF); obstructive sleep apnea (OSA); | Dysrhythmias; |
| | hyponatremia; cardiogenic shock; hyperparathyroidism; s/p ICD; pulm. | |
| | HTN; former smoker (10 pack years) | |
| Heart Failure | Failed- ICM; NSTEMI; LVEF <15% ; on IABP for 10 days prior to Tx; 3rd | Polymorphic |
| Patient 7 | MI; s/p AICD; CAD; PVD; BMI = 28.46; former smoker, quit 2009, 1ppd | Ventricular |
| | | Tachycardia |
| Heart Failure | Failed- ICM; NSTEMI; LVEF <15% ; on IABP for 10 days prior to Tx; 3rd | Polymorphic |
| Patient 8 | MI; s/p AICD; CAD; PVD; BMI = 28.46; former smoker, quit 2009, 1ppd | Ventricular |
| | | Tachycardia |
| Heart Failure | Failed- dilated idiopathic CM; ICM secondary to tachycardia; s/p LVAD | Dysrhythmia |
| Patient 9 | #468 (LVAD explanted in December 2014 due to recovery); cardiogenic shock; | |
| | HTN; exercise tolerance <4METS; fluid volume overload; BMI = 20.35; | |
| | inotrope support (milrinone); moderate AR, severe MR, Pulm HTN; former | |
| | smoker | |
| Heart Failure | Failed- NICM; prediabetic (family history NIDDM); hx acute kidney | Left bundle |
| Patient 10 | injury; leukocytosis; cardiogenic shock; s/p ICD; hypothyroid; chronic | branch block |
| | systolic heart failure; liver shock; nonsmoker; pulm HTN; pre-op IABP and | |
| | inotropes; acute myocarditis (primarily lymphocytes but focal neutrophils | |
| | and rare eosinophils); no CAD; acute hepatitis. | |

TABLE 11
High-resolution transthoracic echocardiography performed on conscious mice
in Groups 1-5 at baseline (before loss of Brg1) and at 1-day pre-mortem.

| | Group 1 | Group 2 | Group 3 | | Group 4 | | Group 4 |
|---|---|---|---|---|---|---|---|
| | Brm−/− | Brm−/− | Brm−/− | Group 5 | Brm−/− | | Brm−/− |
| | flx/flx | flx/flx | flx/flx | Brm−/− | flx/flx | Groups 1-3, 5 | flx/flx |
| | No Tg | No Tg | Brg1 Tg+ | flx/+ | Brg1 Tg+ | (CONTROLS) | Brg1 Tg+ +TAM |
| | Chow Diet | +TAM | Chow | Brg1 Tg+ | +TAM | Day Matched | 1 Day Pre-Mortem |
| | Baseline | Baseline | Baseline | Baseline | Baseline | Pre-Mortem | (Day 11.6 ± 1.5) |
| | N = 3 | N = 14 | N = 5 | N = 7 | N = 30 | N = 28 | N = 27 |
| AWTS (mm) | 1.73 ± 0.10 | 1.77 ± 0.04 | 1.92 ± 0.13 | 1.62 ± 0.04 | 1.84 ± 0.04 | 1.83 ± 0.04 | 1.37 ± 0.10^{§} |
| LVESD (mm) | 1.28 ± 0.04 | 1.28 ± 0.04 | 1.24 ± 0.01 | 1.16 ± 0.08 | 1.29 ± 0.03 | 1.22 ± 0.03 | 3.16 ± 0.24^{§} |
| PWTD (mm) | 1.12 ± 0.03 | 1.09 ± 0.02 | 1.18 ± 0.07 | 0.97 ± 0.05 | 1.06 ± 0.02 | 1.10 ± 0.02 | 1.01 ± 0.06 |
| PWTS (mm) | 1.91 ± 0.06 | 1.78 ± 0.02 | 1.78 ± 0.06 | 1.60 ± 0.06 | 1.68 ± 0.03 | 1.77 ± 0.03 | 1.27 ± 0.07^{§} |
| LV Mass (mg) | 110.9 ± 4.9 | 114.1 ± 3.7 | 126.9 ± 11.1 | 87.9 ± 7.1 | 117.8 ± 5.3 | 111.9 ± 4.0 | 156.0 ± 9.1^{§} |
| LV Mass/BW | 3.1 ± 0.2 | 3.9 ± 0.1 | 4.1 ± 0.4 | 3.9 ± 0.2 | 4.2 ± 0.1 | 3.9 ± 0.1 | 6.2 ± 0.3^{§} |
| (mg/g) | | | | | | | |
| BW | 36.1 ± 0.6 | 29.7 ± 1.2 | 31.2 ± 0.7 | 22.5 ± 0.9 | 27.7 ± 1.0 | 28.8 ± 0.9 | 25.4 ± 0.9 |

Data represent means ± SEM.
A One Way Analysis of Variance was performed, followed by an all pairwise multiple comparison procedure (Holm-Sidak method), when overall significance <0.05.
^{§}p < 0.001 vs. all other groups.
HR, heart rate; ExLVD, external left ventricular diameter; bpm, heart beats per minute; AWTD, anterior wall thickness in diastole; AWTS, anterior wall thickness in systole; PWTD, posterior wall thickness in diastole; PWTS, posterior wall thickness in systole; LVEDD, left ventricular end-diastolic dimension; LVESD, left ventricular end-systolic dimension; FS, fractional shortening, calculated as (LVEDD − LVESD)/LVEDD × 100; EF %, ejection fraction calculated as (end Simpson's diastolic volume − end Simpson's systolic volume)/end Simpson's diastolic volume * 100, ND, not determined.

TABLE 12
Two phenotypic subgroups of Brm/Brg1 double mutant mice at 1-day pre-mortem.
High-resolution transthoracic echocardiography performed on conscious mice 1-day pre-
mortem (and on time-matched controls) after initiating loss of Brg1 via tamoxifen/chow or
chow diet for 7 days. Data represent means ± SEM. A One Way Analysis of Variance was
performed, followed by an all pairwise multiple comparison procedure (Holm-Sidak method),
when overall significance <0.05.

| | | | 2 | | |
|---|---|---|---|---|---|
| | | | Phenotypic | | Subset 2. |
| | | Group 4 | Subsets | Subset 1. | Group 4 |
| | | Brm−/− flx/flx | Group 4 | Group 4 | Brm/Brg1 |
| | | Brg1 Tg+ | Brm/Brg1 | Brm/Brg1 | Double |
| | Groups 1-3, 5 | +TAM | Double | Double Mutants | Mutants |
| | (CONTROLS) | 1 Day Pre- | Mutants | EF % <50% | EF >50% |
| | Day Matched | Mortem | 1. <50% EF | 1 Day Pre- | 1 Day Pre- |
| | Pre-Mortem | (Day 11.6 ± 1.5) | 2. >50% EF | Mortem | Mortem |
| | N = 28 | N = 27 | → | N = 17 | N = 10 |
| AWTS (mm) | 1.83 ± 0.04§ | 1.37 ± 0.10 | → | 1.14 ± 0.11** | 1.87 ± 0.13§ |
| LVESD (mm) | 1.22 ± 0.03 | 3.16 ± 0.24**,† | → | 4.17 ± 0.37* | 1.84 ± 0.21 |
| PWTD (mm) | 1.10 ± 0.02 | 1.01 ± 0.06 | → | 0.93 ± 0.07† | 1.23 ± 0.11 |
| PWTS (mm) | 1.77 ± 0.03* | 1.27 ± 0.07§ | → | 1.17 ± 0.07† | 1.56 ± 0.10 |
| LV Mass (mg) | 111.9 ± 4.0* | 156.0 ± 9.1 | → | 150.2 ± 15.0 | 148.0 ± 13.6 |
| LV Mass/BW | 3.9 ± 0.1* | 6.2 ± 0.3 | → | 6.0 ± 0.3 | 6.4 ± 0.7 |
| (mg/mm) | | | | | |
| BW | 28.8 ± 0.9 | 25.4 ± 0.9 | → | 26.4 ± 1.1 | 23.7 ± 1.5** |

*p < 0.001 vs. all other groups;
**p < 0.05 vs. Column 1;
§p < 0.05 vs. Column 2;
†p < 0.01 vs. Column 4.
HR, heart rate;
ExLVD, external left ventricular diameter;
bpm, heart beats per minute;
AWTD, anterior wall thickness in diastole;
AWTS, anterior wall thickness in systole;
PWTD, posterior wall thickness in diastole;
PWTS, posterior wall thickness in systole;
LVEDD, left ventricular end-diastolic dimension;
LVESD, left ventricular end-systolic dimension;
FS, fractional shortening, calculated as (LVEDD − LVESD)/LVEDD × 100;
EF % , ejection fraction calculated as (end Simpson's diastolic volume − end Simpson's systolic volume)/end Simpson's diastolic volume * 100,
ND, not determined.

1. A method of increasing expression of Brg1 and/or Brm mRNA and/or BRG1 and/or BRM protein in a subject, comprising delivering to the subject an effective amount of a histone deacetylase (HDAC) inhibitor and/or a histone acetyltransferase (HAT) activator, thereby increasing expression of Brg1 and/or Brm mRNA and/or BRG1 and/or BRM protein in the subject.

2. A method of decreasing expression of c-Myc mRNA and/or c-Myc protein in a subject, comprising delivering to the subject an effective amount of a histone deacetylase (HDAC) inhibitor and/or a histone acetyltransferase (HAT) activator, thereby decreasing expression of c-Myc mRNA and/or c-Myc protein in the subject.

3. A method of increasing expression of Cx40, Cx43, and/or Scn5a mRNA and/or Cx40, Cx43, and/or SCN5A protein in a subject, comprising delivering to the subject an effective amount of a histone deacetylase (HDAC) inhibitor and/or a histone acetyltransferase (HAT) activator, thereby increasing expression of Cx40, Cx43, and/or Scn5a mRNA and/or Cx40, Cx43, and/or SCN5A protein in the subject.

4.-27. (canceled)

28. The method of claim 1, wherein the HDAC inhibitor is a short-chain fatty acid, a hydroxamic acid, a cyclic tetrapeptide, a benzamide, a tricyclic lactam, a sultam derivative, an organosulfur compound; a electrophilic ketone, pimeloylanilide o-aminoanilide (PAOA), depudecin, a psammaplin, Vorinostat, tubacin, curcumin, histacin, 6-Chloro-2,3,4,9-tetrahydro-1H-carbazole-l-carboxamide, CRA-024781, CRA-026440, CG1521, PXD101, G2M-777, CAY10398, CTPB MGCDO103, CUDC-100, and/or any derivative thereof or combination thereof.

29. The method of claim 28, wherein the short-chain fatty acid is butyrate, phenylbutyrate, pivaloyloxymethyl butyrate, N-Hydroxy-4-(3-methyl-2-phenyl-butyrylamino)-benzamide,4-(2,2-Dimethyl-4-phenylbutyrylamino)-N-hydroxybenzamide, valproate, valproic acid, and/or any derivative thereof or combination thereof.

30. The method of claim 28, wherein the hydroxamic acid is suberoylanilide hydroxamic acid (SAHA), oxamflatin, M-carboxycinnamic acid bishydroxamide, suberic bishydroxamate (SBHA), nicotinamide, scriptaid (SB-556629), scriptide, splitomicin, lunacin, ITF2357, A-161906, NVP-LAQ824, LBH589, pyroxamide, Panobinostat (LB589), givinostat (or gavinostat (originally ITF2357)), resminostat (RAS2410), CBHA, 3-C1-UCHA, SB-623, SB-624, SB-639, SK-7041, a propenamide, an aroyl pyrrolyl hydroxyamide, a trichostatin, and/or any derivative thereof or combination thereof.

31. The method of claim 30, wherein the propenamide is MC 1293 and/or any derivative thereof, the aroyl pyrrolyl hydroxyamide is APHA Compound 8 and/or any derivative thereof, and the trichostatin is trichostatin A, trichostatin C, and/or any derivative thereof or combination thereof.

32. The method of claim 28, wherein the cyclic tetrapeptide is a trapoxin, romidepsin, HC-toxin, chlamydocin, diheteropeptin, WF-3161, Cyl-1, Cyl-2, apicidin, depsipeptide (FK228), FR225497, FR901375, a spiruchostatin, a salinamide, a cyclic-hydroxamic-acid-containing peptide, and/or any derivative thereof or combination thereof.

33. The method of claim 32, wherein the spiruchostatin is spiruchostatin A, spiruchostatin B, spiruchostatin C, and/or any derivative thereof, and the salinamide is salinamide A, salinamide B and/or any derivative thereof or combination thereof.

34. The method of claim 28, wherein the benzamide is M344, MS-275, CI-994 (N-acetyldinaline), tacedinaline, sirtinol, and/or any derivative thereof or combination thereof.

35. The method of claim 28, wherein the organosulfur compound is diallyl disulfide, sulforaphane; and/or any derivative thereof or combination thereof.

36. The method of claim 28, wherein the electrophilic ketone is α-ketoamide, trifluoromethylketone and/or any derivative thereof or combination thereof.

37. The method of claim 1, wherein the HAT activator is benzamide, N-[4-chloro-3-(trifluoromethyl)phenyl]-2-ethoxy-6-pentadecyl (CTPB), nemorosome or TTK21, or any combination thereof.

38. The method of claim 1, wherein the subject is a human.

39. The method of claim 1, wherein the subject is an animal model of cardiac conductance defect, arrhythmia, heart failure, familial hypertrophic cardiomyopathy and/or long QT syndrome.


SEQUENCE LISTING (141 sequences)

SEQ ID NO: 1 - DNA, 19 bp, Artificial
  primer
tgtccgtcgtggatctgac

SEQ ID NO: 2 - DNA, 20 bp, Artificial
  primer
cctgcttcaccaccttcttg

SEQ ID NO: 3 - DNA, 20 bp, Artificial
  primer
ctaaggccaaccgtgaaaag

SEQ ID NO: 4 - DNA, 20 bp, Artificial
  primer
accagaggcatacagggaca

SEQ ID NO: 5 - DNA, 18 bp, Artificial
  primer
tacctgctggctggatgg

SEQ ID NO: 6 - DNA, 20 bp, Artificial
  primer
cacagcctcggcatatttct

SEQ ID NO: 7 - DNA, 18 bp, Artificial
  primer
ttaaatgggccagcgaag

SEQ ID NO: 8 - DNA, 20 bp, Artificial
  primer
tgctctgccatgatgttttc

SEQ ID NO: 9 - DNA, 20 bp, Artificial
  primer
ctttctcgactggtgaggaa

SEQ ID NO: 10 - DNA, 20 bp, Artificial
  primer
gagcctgttagttgctcccg

SEQ ID NO: 11 - DNA, 20 bp, Artificial
  primer
cccttctcgtcagcacattg

SEQ ID NO: 12 - DNA, 20 bp, Artificial
  primer
agccactgactcaactggaa

SEQ ID NO: 13 - DNA, 17 bp, Artificial
  primer
gtcagagtggtgggctg

SEQ ID NO: 14 - DNA, 18 bp, Artificial
  primer
gatccccacatcccacgg

SEQ ID NO: 15 - DNA, 21 bp, Artificial
  primer
tacgttgtctcatttcgaagg

SEQ ID NO: 16 - DNA, 22 bp, Artificial
  primer
catttttggtcttgtggtcttg

SEQ ID NO: 17 - DNA, 27 bp, Artificial
  primer
catctgcttattaccagctaccaccca

SEQ ID NO: 18 - DNA, 20 bp, Artificial
  primer
gcctgccgaaagagtcagag

SEQ ID NO: 19 - DNA, 21 bp, Artificial
  primer
tctcattcttcagctcctcaa

SEQ ID NO: 20 - DNA, 25 bp, Artificial
  primer
tgggccttcagctcagcattcacac

SEQ ID NO: 21 - DNA, 20 bp, Artificial
  primer
tccgagccatgagaaacaag

SEQ ID NO: 22 - DNA, 19 bp, Artificial
  primer
gagcccagcgtctcctgaa

SEQ ID NO: 23 - DNA, 23 bp, Artificial
  primer
accactaccgggagctccccgtg

SEQ ID NO: 24 - DNA, 19 bp, Artificial
  primer
gttccagaggtggaggcca

SEQ ID NO: 25 - DNA, 20 bp, Artificial
  primer
tagtctgagtgctgcggact

SEQ ID NO: 26 - DNA, 20 bp, Artificial
  primer
gcctgcacctgactcagcag

SEQ ID NO: 27 - DNA, 23 bp, Artificial
  primer
acccggccaccagccttactcca

SEQ ID NO: 28 - DNA, 22 bp, Artificial
  primer
cgaagaggaagaatcaaaaacc

SEQ ID NO: 29 - DNA, 19 bp, Artificial
  primer
tgtgacctctgttggccct

SEQ ID NO: 30 - DNA, 25 bp, Artificial
  primer
actactcttgaccctgcgtccctag

SEQ ID NO: 31 - DNA, 19 bp, Artificial
  primer
cctcggaatggccggctat

SEQ ID NO: 32 - DNA, 19 bp, Artificial
  primer
ggaaaaggcatcctccttg

SEQ ID NO: 33 - DNA, 24 bp, Artificial
  primer
atgatggcttggccagtgcctcag

SEQ ID NO: 34 - DNA, 20 bp, Artificial
  primer
ataaaccccgatgaggctgt

SEQ ID NO: 35 - DNA, 20 bp, Artificial
  primer
accagatcacctgtatcctg

SEQ ID NO: 36 - DNA, 23 bp, Artificial
  primer
ctatggtgccgctgtccaggctg

SEQ ID NO: 37 - DNA, 19 bp, Artificial
  primer
ctgacgggttctcggttga

SEQ ID NO: 38 - DNA, 20 bp, Artificial
  primer
atcagtggttcaccgcttct

SEQ ID NO: 39 - DNA, 25 bp, Artificial
  primer
ctgagaagtcacgttctgacaggac

SEQ ID NO: 40 - DNA, 21 bp, Artificial
  primer
caccgagatttccttcaaact

SEQ ID NO: 41 - DNA, 20 bp, Artificial
  primer
gccatctagggttatgatgc

SEQ ID NO: 42 - DNA, 26 bp, Artificial
  primer
cgtggaattcgatgaaatcacgcgca

SEQ ID NO: 43 - DNA, 20 bp, Artificial
  primer
tctgcgaaatagacgttcgg

SEQ ID NO: 44 - DNA, 20 bp, Artificial
  primer
ttcgtactggcctttctcca

SEQ ID NO: 45 - DNA, 23 bp, Artificial
  primer
ctcaaccccctgcgccaacaatg

SEQ ID NO: 46 - DNA, 19 bp, Artificial
  primer
cagcaacgagtaccgggta

SEQ ID NO: 47 - DNA, 19 bp, Artificial
  primer
tgcgtctccacgttgcgtt

SEQ ID NO: 48 - DNA, 26 bp, Artificial
  primer
caacaacatcgcggtgcgcaagagcc

SEQ ID NO: 49 - DNA, 20 bp, Artificial
  primer
ctgagcgacgagtacaagat

SEQ ID NO: 50 - DNA, 18 bp, Artificial
  primer
gcgtctccaggttgcgca

SEQ ID NO: 51 - DNA, 20 bp, Artificial
  primer
agagcatgaccgatggattc

SEQ ID NO: 52 - DNA, 20 bp, Artificial
  primer
attaggcgcaggaaggtcag

SEQ ID NO: 53 - DNA, 20 bp, Artificial
  primer
ctccgaccccgccgatgtcg

SEQ ID NO: 54 - DNA, 20 bp, Artificial
  primer
agaaacggctaccacatcca

SEQ ID NO: 55 - DNA, 21 bp, Artificial
  primer
ctcgaaagagtcctgtattgt

SEQ ID NO: 56 - DNA, 22 bp, Artificial
  primer
aggcagcaggcgcgcaaattac

SEQ ID NO: 57 - DNA, 20 bp, Artificial
  primer
aggtcggtgtgaccggattt

SEQ ID NO: 58 - DNA, 20 bp, Artificial
  primer
ggcaacaatctccactttgc

SEQ ID NO: 59 - DNA, 24 bp, Artificial
  primer
tgcaaatggcagccctggtgacca

SEQ ID NO: 60 - DNA, 18 bp, Artificial
  primer
ctgcctgacggccaggtc

SEQ ID NO: 61 - DNA, 22 bp, Artificial
  primer
caagaaggaaggctggaaaaga

SEQ ID NO: 62 - DNA, 24 bp, Artificial
  primer
cactattggcaacgagcggttccg

SEQ ID NO: 63 - DNA, 24 bp, Artificial
  primer
caagaagacattagtgagatatgg

SEQ ID NO: 64 - DNA, 23 bp, Artificial
  primer
gcaagaagatcaaatagcaaacc

SEQ ID NO: 65 - DNA, 27 bp, Artificial
  primer
atatgaaggcacaccactgaaatggca

SEQ ID NO: 66 - DNA, 20 bp, Artificial
  primer
agcagcagtgacgatcggat

SEQ ID NO: 67 - DNA, 20 bp, Artificial
  primer
cgtgaaagaaatccccggat

SEQ ID NO: 68 - DNA, 23 bp, Artificial
  primer
tgagcctccaacctctcttgggc

SEQ ID NO: 69 - DNA, 22 bp, Artificial
  primer
tatgtgaatcagtcctttgctc

SEQ ID NO: 70 - DNA, 20 bp, Artificial
  primer
ccagtttaccatcactgcca

SEQ ID NO: 71 - DNA, 25 bp, Artificial
  primer
tccccagaccaagaagttggaactc

SEQ ID NO: 72 - DNA, 20 bp, Artificial
  primer
gcatatggcatgatagcctc

SEQ ID NO: 73 - DNA, 21 bp, Artificial
  primer
tccacatggcattaatctcct

SEQ ID NO: 74 - DNA, 24 bp, Artificial
  primer
atcaccagtatcttcagccccagg

SEQ ID NO: 75 - DNA, 23 bp, Artificial
  primer
gcacttgaaccagataaaactgt

SEQ ID NO: 76 - DNA, 20 bp, Artificial
  primer
gcatgtaatgcacagcctct

SEQ ID NO: 77 - DNA, 27 bp, Artificial
  primer
tccgacaggtctaagcggaatttatcc

SEQ ID NO: 78 - DNA, 22 bp, Artificial
  primer
gacgccttattcttctttgtca

SEQ ID NO: 79 - DNA, 21 bp, Artificial
  primer
ctcatgattgtcctcatacag

SEQ ID NO: 80 - DNA, 25 bp, Artificial
  primer
accatccctcccaccagtgctacca

SEQ ID NO: 81 - DNA, 22 bp, Artificial
  primer
gcttctgataatcaaccaactg

SEQ ID NO: 82 - DNA, 21 bp, Artificial
  primer
tgtacccagaagatgattgtc

SEQ ID NO: 83 - DNA, 26 bp, Artificial
  primer
aggtctatgaaggtgaaagacccctg

SEQ ID NO: 84 - DNA, 20 bp, Artificial
  primer
gaagaggaggcggaagccaa

SEQ ID NO: 85 - DNA, 19 bp, Artificial
  primer
gcctgcacctgactcagca

SEQ ID NO: 86 - DNA, 23 bp, Artificial
  primer
acccggccactggcctcacttca

SEQ ID NO: 87 - DNA, 18 bp, Artificial
  primer
aggctcgccatgccggga

SEQ ID NO: 88 - DNA, 18 bp, Artificial
  primer
gctccgcctcgtagaagt

SEQ ID NO: 89 - DNA, 24 bp, Artificial
  primer
actctaactcccccatggagtcgg

SEQ ID NO: 90 - DNA, 21 bp, Artificial
  primer
ataaataaccgggctcaggag

SEQ ID NO: 91 - DNA, 18 bp, Artificial
  primer
cctcgggtgggtcccctt

SEQ ID NO: 92 - DNA, 23 bp, Artificial
  primer
ttagcgagtcagagccgcgcacg

SEQ ID NO: 93 - DNA, 22 bp, Artificial
  primer
ctggaaatgaagaggaagaatc

SEQ ID NO: 94 - DNA, 19 bp, Artificial
  primer
tggttctggctcctcctca

SEQ ID NO: 95 - DNA, 26 bp, Artificial
  primer
tcaccactcttgaccctgcttctctg

SEQ ID NO: 96 - DNA, 20 bp, Artificial
  primer
tgcctgcagaaagagtcgga

SEQ ID NO: 97 - DNA, 20 bp, Artificial
  primer
cttctcgttcttgagctcct

SEQ ID NO: 98 - DNA, 26 bp, Artificial
  primer
ctgagccttcagttcagcattcacac

SEQ ID NO: 99 - DNA, 20 bp, Artificial
  primer
acctcaactacatggtttac

SEQ ID NO: 100 - DNA, 20 bp, Artificial
  primer
gaagatggtgatgggatttc

SEQ ID NO: 101 - DNA, 20 bp, Artificial
  primer
caagcttcccgttctcagcc

SEQ ID NO: 102 - DNA, 19 bp, Artificial
  primer
ccagccctgagcccctagt

SEQ ID NO: 103 - DNA, 20 bp, Artificial
  primer
tgctcttcttcagagtcgct

SEQ ID NO: 104 - DNA, 24 bp, Artificial
  primer
tgcatgaggagacaccgcccacca

SEQ ID NO: 105 - DNA, 20 bp, Artificial
  primer
ccactgtaccaagaggaaag

SEQ ID NO: 106 - DNA, 20 bp, Artificial
  primer
tgtctccatgtacggcttct

SEQ ID NO: 107 - DNA, 25 bp, Artificial
  primer
aatgttccagcacggagcaccccta

SEQ ID NO: 108 - DNA, 20 bp, Artificial
  primer
accatcatgggcatgatctg

SEQ ID NO: 109 - DNA, 20 bp, Artificial
  primer
ataggtgaccctgccaagac

SEQ ID NO: 110 - DNA, 26 bp, Artificial
  primer
tgatcgtggaggtcttgctgaggatg

SEQ ID NO: 111 - DNA, 21 bp, Artificial
  primer
cctcttcaagtctgtcttcga

SEQ ID NO: 112 - DNA, 20 bp, Artificial
  primer
tagaccgcactcaggctgaa

SEQ ID NO: 113 - DNA, 25 bp, Artificial
  primer
tggccttcctgctgatccagtggta

SEQ ID NO: 114 - DNA, 20 bp, Artificial
  primer
attcccttcgttcctgtacc

SEQ ID NO: 115 - DNA, 20 bp, Artificial
  primer
actgggagaactctcctcca

SEQ ID NO: 116 - DNA, 25 bp, Artificial
  primer
acgaggcgagcctgtcacagtgtac

SEQ ID NO: 117 - DNA, 20 bp, Artificial
  primer
tcaccaacagctggaacatc

SEQ ID NO: 118 - DNA, 18 bp, Artificial
  primer
ggagagacagtgccaacg

SEQ ID NO: 119 - DNA, 20 bp, Artificial
  primer
agagcaacgtgcgaacaacg

SEQ ID NO: 120 - DNA, 21 bp, Artificial
  primer
caacacctgacatgtacgcat

SEQ ID NO: 121 - DNA, 25 bp, Artificial
  primer
cgatttcgtggttgtcatcctctcc

SEQ ID NO: 122 - DNA, 19 bp, Artificial
  primer
cgaaaggagctgcccgagt

SEQ ID NO: 123 - DNA, 19 bp, Artificial
  primer
tggttgcgaatgcgctcct

SEQ ID NO: 124 - DNA, 23 bp, Artificial
  primer
acgagctcatccgcaagcccgtg

SEQ ID NO: 125 - DNA, 20 bp, Artificial
  primer
gccgtgacgtggactacagt

SEQ ID NO: 126 - DNA, 20 bp, Artificial
  primer
aaattgccgtcttcgatggc

SEQ ID NO: 127 - DNA, 23 bp, Artificial
  primer
acgccctcagggagaagcagtgg

SEQ ID NO: 128 - DNA, 20 bp, Artificial
  primer
ttcgggtagtggaaaaccag

SEQ ID NO: 129 - DNA, 20 bp, Artificial
  primer
ggtcatagttcctgttggtg

SEQ ID NO: 130 - DNA, 23 bp, Artificial
  primer
tcccgcgacgatgcccctcaacg

SEQ ID NO: 131 - DNA, 22 bp, Artificial
  primer
ccaacagctggaatatcttcga

SEQ ID NO: 132 - DNA, 20 bp, Artificial
  primer
ttctggatgatgtccgagag

SEQ ID NO: 133 - DNA, 26 bp, Artificial
  primer
tcgtggttgtcatcctctccatcgtg

SEQ ID NO: 134 - DNA, 20 bp, Artificial
  primer
tctttatgctggctgtggct

SEQ ID NO: 135 - DNA, 20 bp, Artificial
  primer
gatcttcttccagcccaggt

SEQ ID NO: 136 - DNA, 24 bp, Artificial
  primer
actgtccctcctccttagcctggc

SEQ ID NO: 137 - DNA, 20 bp, Artificial
  primer
aagcaaaagagtggtgccca

SEQ ID NO: 138 - DNA, 20 bp, Artificial
  primer
cagcagttgagtaggcttga

SEQ ID NO: 139 - DNA, 26 bp, Artificial
  primer
tgtcaaggagtttgcctaaggcgctc

SEQ ID NO: 140 - DNA, 20 bp, Artificial
  primer
gagtgcaagaccacgttcaa

SEQ ID NO: 141 - DNA, 20 bp, Artificial
  primer
ggagctgatgctttgagacc